(19) United States
(12) Patent Application Publication
(10) Pub. No.: US 2004/0170601 A1
(43) Pub. Date: Sep. 2, 2004

(54) Method of improving the growth performance of an animal

(76) Inventors: Alan David Greve Strom, Victoria (AU); Marion Elizabeth Andrew, Victoria (AU); Aleta Gai Knowles, Enmore (AU)

Correspondence Address: VENABLE, BAETJER, HOWARD AND CIVILETTI, LLP, P.O. BOX 34385, WASHINGTON, DC, 20043-9998, US

(21) Appl. No.: 10/468,891

(22) PCT Filed: Feb. 26, 2002
(86) PCT No.: PCT/AU02/00209

(30) Foreign Application Priority Data
Feb. 26, 2001 (AU) PR 3354
Mar. 5, 2001 (AU) PR 3532
Dec. 18, 2001 (AU) PR 9596

(51) Int. Cl. A61K 38/19

(52) U.S. Cl. 424/85.1

(57) ABSTRACT

The invention broadly relates to a method of improving the growth performance of an animal. In particular the present invention relates to a method of improving the growth performance of an animal comprising the step of administering to an animal in need thereof a growth promoting amount of one or more cytokines or biologically active fragments thereof.

### FIELD OF THE INVENTION

The invention broadly relates to a method of improving the growth performance of an animal. In particular the present invention relates to a method of improving the growth performance of an animal comprising the step of administering to an animal in need thereof a growth promoting amount of one or more cytokines or biologically active fragments thereof.

### BACKGROUND OF THE INVENTION

With an ever-increasing world demand for food, there is constant pressure to increase the rate of production of food. A goal of animal husbandry is to produce feed animals that consistently meet specified industry standards with minimum commercial expenditure.

To achieve this goal, the husbandry environment must be such that the conditions provided therein are biased towards achieving an acceptable growth performance. In other words, the conditions must be sufficient to allow an acceptable growth rate (the rate of unit gain in live weight), an acceptable efficiency of feed use (the amount of feed required per unit gain in live weight) and an acceptable final weight, so that at slaughter, each carcass is characterised by a dressed weight and fat content which meets a specified industry standard.

In the early 1950's, researchers unexpectedly discovered that an antibiotic ingredient in chicken mash was a “growth factor.” The finding drastically changed the livestock and poultry industries and was an economic boon for pharmaceutical companies. Feed animals are now raised under highly controlled conditions and receive specialised feed with a variety of growth promoting additives.

Routine antibiotic administration to animals has become almost universal since the discovery that the addition of small amounts of antibiotics such as penicillin, tetracycline and sulfamethasine, to animal feed increases the growth of pigs and cattle. In 1979, about 70% of the beef cattle and veal, 90% of the swine, and virtually 100% of broilers reared in the United States consumed antibiotics as part of their daily feed. This use, accounting for nearly 40% of antibiotics sold in the United States, is estimated to save consumers $3.5 billion a year in food costs.

Animals raised under modern conditions optimised for growth promotion receive rations containing high proportions of protein, usually in the form of soybean or cottonseed meal (meat and bone or blood meal are used extensively in Australia), and high percentages of grains such as corn or milo, a type of sorghum (wheat and barley in Australia). Feed additives which have been used include such hormones as diethyl-stilbesterol, which also increases the rate of weight gain, and tranquillisers (not used widely for pigs) that prevent the effects of the stress brought on by confinement conditions from causing disease or weight loss.

Cattle ordinarily require 5 kilograms of feed to produce 1 kilogram of weight gain. Under optimal growth promoting conditions, and with enriched feed, they gain 1 kilogram with only 3 kilograms of feed.

Although hormones and antibiotics have greatly increased the rate of growth of food animals, the use of such additives has not been without problems. One of the hormones that is commonly used as a growth stimulant, diethyl-stilbesterol or DES, has been shown to be a carcinogen and has been banned from further use in most countries.

When antibiotics are mixed in animal feed, the compounds are spread throughout the environment exposing microorganisms to the antibiotics. The constant exposure of the microorganisms to antibiotics puts biological pressure on the microorganisms to develop a resistance to the antibiotics. This can result in a microorganism that is resistant to antibiotics and causes especially severe and difficult to treat infections.

An antibiotic-resistant microorganism is potentially a serious pathogen because it is difficult to control. If the organism causes an infection in an animal or in man, the infection may not be controlled with conventional antibiotics. If the infection is serious, there may not be time to determine which antibiotics are effective against the infecting bacteria. The problem has been especially serious when antibiotic resistant organisms in meat are consumed by people who themselves take antibiotics for treatment of disease. Antibiotics inhibit many of the normal microorganisms in the respiratory and gastrointestinal tracts. This allows the resistant one to proliferate rapidly and produce more serious disease. The combination of antibiotic resistant organisms from food and ineffective antibiotic treatment of people has caused most of the deaths due to salmonella food poisoning reported in the United States in the past several years.

As a result of the increasing appearance of antibiotic resistant bacteria in feed lots and several serious epidemics caused by antibiotic resistant bacteria, there is increasing governmental pressure to ban the use of antibiotics in animal feed. In fact, the World Health Organisation and the Australian Government have specified the need to use environmentally friendly alternative methods to control infection. The imminent ban or withdrawal of various antibiotics from livestock feed and water is likely to (i) increase the incidence of infection in animals and consequently reduce growth performance (ii) further reduce the health, fertility and breeding performance of animals. Consequently, there is an immediate and increasing need for new, safe and effective growth stimulators of feed animals, as well as a reduction in disease by enhancing health.

Various attempts at promoting animal growth without the use of antibiotics has been employed many using elaborate and circuitous means. These have included subcutaneous implants of hormones or complex salts having cations being made from complexes (see, for example, U.S. Pat. No. 6,197,815; U.S. Pat. No. 3,991,750; U.S. Pat. No. 4,067,994). None of these attempts have proven to be simple or effective. Accordingly, there is still a need for a method of improving the growth performance of animals, which is not reliant on the use of antibiotics or elaborate methodology.

The applicant has now surprisingly found that the administration of certain cytokines, and in particular interleukins, increases the growth performance of animals while decreasing the amount of antibiotics required.

While not wishing to be bound by any particular theory or hypothesis, the applicant considers that the increases in growth performance observed in animals that have been administered cytokines results from the interplay of four key effects. These are:

1). Immunoenhancement effect;

2). Anti-parasitic and anti-microbial effect;

3). Stress reduction; and

4). Anti-inflammatory effect.

Each of these effects, either singly or together, profoundly impact upon the health and welfare of animals, which in turn affects the growth performance of animals and thereby the meat quality. For example:

### 1). Immunoenhancement Effect

a). TH1/TH2 Immune Responses

Interplay occurs between immune cytokine regulatory networks and the other regulatory systems of the body. Immune responses to infections or antigens can acutely bias each other. The immune response can be generalised by the type of T cell response. A T helper 1 (TH1) type response is principally involved in cell mediated immunity, whilst a TH2 pattern of response is often associated with humoral immunity. TH1 and TH2 type T cell subsets have been implicated in the regulation of many immune responses defined by cytokine patterns. TH2 cells express the cytokines interleukin (IL)-4, IL-5, IL-10, and IL-13. IL-3 expression is common to both TH1 and TH2 T cells. Whereas, TH1 cells express IL-2, IFNgamma, and TNFbeta. These TH2 cytokines influence B cell development and augment humoral responses such as the secretion of antibodies. Both types of TH cells influence each other by the cytokines they secrete. For example, TH2 cytokines, such as IL-10, can suppress TH1 functions. Other cytokines can also influence TH1 or TH2 development such as TGFbeta, known to down regulate TH1 responses. Cytokines regulating TH2 responses may influence immune parameters resulting in increased health or productivity.

b). Antibody Isotype Switching

Antibodies are required to eliminate or protect against infection. Mature B cells undergo the process of switching antibody class after antigenic stimulation. TH cells through physical contact and cytokines, referred to as switch factors, regulate isotype switching. Some of the cytokines known to be involved in isotype switching, either alone or in combination, are IL-4, IL-5, TGFbeta, IL-1, IL-2, IL-6, and IL-13. IL-4 and IL-5 synergise to enhance IgG1 responses. For example, optimal IgG1 responses also requires IL-2. IL-1 can enhance IgA production in the presence of IL-5. TGFbeta induces IgA production.

c). Hematopoiesis

Hematopoiesis is the process of blood cell formation including red blood cells and the immune cells (white blood cells). Bone marrow is the major source of post-natal generation of new blood cells. Hematopoietic growth factors are required for the maintenance of this process to maintain hematopoietic stem cells, their proliferation, differentiation and maturation into different lineages critical for the immune system. The hematopoietic growth factors include various colony stimulating factors (such as IL-3), Epo, SCF, various interleukins (IL-1, IL-3, IL-4, IL-5, IL6, IL-11, IL-12), LIF, TGFbeta, MIP1alpha, TNFalpha. Many of these factors are multifunctional.

d). Immune Dysfunction

The genetic potential for most production traits is predetermined by birth. Many factors (stress, disease, nutrition, immunity etc.) determine whether this potential is achieved. The level and type of antigen exposure influences and establishes a ‘bias’ of the immune system. Most immune responses are biased towards a type that promotes immunity against bacteria and viruses or a type that promotes immunity against many parasites. While the genotype of an animal can influence this bias, the early experience by the neonate to antigens and infections can set the immune reactivity towards one or other type. This bias is altered depending on subsequent antigen exposure. Breeding programmes based on selection for production traits has appeared to be at the expense and detriment of immune competence or reactivity. This change has been further exacerbated by the persistent use of antibiotic supplements to water and feed, which has presumably resulted in an altered genetic potential to mount effective immune responses.

e). Mucosal Immunity

The most prevalent areas of infection in livestock are mucosal sites, primarily the gastro-intestinal tract and the lungs. Thus, the mucosal immune system is the first line of defence against pathogens and disease. Cytokines, notably IL-5, IL-4, IL-6 and IL-10, play a significant role in the regulation and efficacy of immune responses in the mucosa.

IL-5 and IL-6 act upon B-1 and B-2 subpopulations of lymphocytes in the mucosal immune system. Deficiencies in either the production of IL-5 or IL-6, or their receptors result in significantly impaired production of IgA, the antibody isotype responsible for protective responses in the mucosa. Similarly, IL-5, IL-6 and the chemokine MIP-1 alpha have the capacity to increase IgA responses to mucosal vaccines. IL-4 has an immunoregulatory role in mucosal tissues, primarily by enhancing TH2 responses, and thus, enhancing antibody production. IL-4 is considered essential to the development of mucosal immune responses in the lung, via the involvement of TH2 pathways. Both IL-4 and IL-5 operate in concert in the lung, with IL-4 committing naive T cells to a TH2 phenotype which upon subsequent activation secrete IL-5, resulting in eosinophil accumulation. Furthermore, IL-4 and IL-10 play a role in mucosal tolerance, and thus, help regulate and dampen allergic type responses in the gut and reduce the susceptibility of animals to chronic inflammatory conditions of the gut.

Delivery of cytokines such as IL-4, IL-5 and IL-10 may enhance the resistance of animals to mucosal pathogens, even at the subclinical level, thereby reducing the deleterious effects of subclinical disease on growth and productivity of livestock. By improving mucosal immunity, disease prevalence and the associated costs of treatment and prophylactic use of antibiotics may also be reduced.

### 2). Anti-Parasitic and Anti-Microbial Effect

a). Anti-parasitic Effect

Acquired immune responses against pathogens generally fall into one of two types, cell mediated (TH1) or antibody mediated (TH2), and this is controlled by cytokines. Cytokines involved in TH2 responses are attractive therapeutic targets, as they could protect against ectoparasites and gastrointestinal worms and suppress inflammation induced by TH1 cytokines. TH2 cytokines induce eosinophilia, IgE synthesis, and mucus production that enhance protection against worms and other gut parasites. Therefore, cytokines, such as IL-3, IL-4, IL-5, IL-6 and GM-CSF, which are important in the development of protective mucosal immune responses and are capable of inducing eosinophilia, are potential candidates in the control of parasitic infections.

b). Anti-microbial Effects

Microbial infections remain a world-wide problem in terms of economic impacts and health, despite advances in nutrition, vaccines, chemicals and antibiotics. The immune response to microbial pathogens incorporates two systems of recognition. The first line of defence is innate immunity and this is followed, if required, by the ensuing adaptive response (cell mediated and antibody responses). Cells such as phagocytes chiefly carry out the innate immune response. Cytokines such as IL-6, IL-15, IL-18 are made by innate cells early in the response to infection and other cytokines regulate their development and function such as GM-CSF, G-CSF, SCF, IL-3, SCF, IL-6, IL-1, IL-4, IL-5. These cytokines maybe critical to the early detection of pathogens and the direction of protective immune responses, specifically reducing the duration and severity of infection as well as the rate of new infections especially by pre-treatment or continuous treatment with cytokines.

### 3). Stress Reduction

Many conditions within a commercial environment contribute to a reduction in feed intake, growth rate and carcass quality. Despite extensive research efforts to evaluate the mechanisms by which stressors affect performance in many species; the long-standing problems within the livestock industries have not been alleviated. Stress, particularly early and sustained stress, results in immune dysfunction, Hypothalamic-Pituitary-Adrenalcortical (HPA) activity and an imbalance of chemicals in the brain. The nervous and immune systems are integrated and form an interdependent neuroimmune network. Depression, physical or emotional stresses activate the endocrine system altering immunological function, which in turn elicits physiological and chemical changes in the brain. Cytokines mediate interactions between the immune, endocrine and central nervous systems. Previously believed to be immuno-suppressive, there is mounting evidence that stress induces a shift in TH1/TH2 immune responses resulting in immune dysregulation rather than immunosuppression. The potential for cytokines to affect homeostatic pathways creates a need to evaluate the activities of the immune system.

### 4. Anti-Inflammation

Chronic inflammation is often seen in livestock and relates to immune activation triggered by persistent infections and environmental stimuli. Inflammation plays an important role in the initiation of immune responses to infection, however, chronic immune activation, particularly by persistent infection or microbial load, can have deleterious effects on growth and development and can reduce the effectiveness of vaccination. Consequences of excessive immune activation include the production of inflammatory cytokines, fever, inappetence, amino acid resorption from muscle and redirection of nutrients away from meat production. Cytokines with anti-inflammatory function could reduce the pathology of chronic immune activation. This could include cytokines like IL-4 and IL-10.

### SUMMARY OF THE INVENTION

In its broadest aspect the present invention provides a method for improving the growth performance of an animal comprising the step of administering to an animal in need thereof a growth promoting amount of one or more cytokines or biologically-active fragments thereof.

The present invention also provides a method for improving the growth performance of an animal comprising the step of administering to an animal in need thereof a compound or composition which increases or supplements endogenous cytokine levels such that a growth promoting amount of one or more cytokines is produced, wherein growth performance is enhanced relative to the growth performance of an animal which has not been administered said compound or composition.

Preferably, the compound or composition is administered prior to, together with, or subsequent to the administration of a growth promoting amount of one or more cytokines.

The present invention also provides a method for improving the growth performance of an animal comprising the step of administering to an animal in need thereof a composition comprising a cytokine or biologically-active fragment thereof in conjunction with an antibiotic, optionally in combination with a pharmaceutical carrier, adjuvant or vehicle, wherein said composition achieves a synergistic growth promoting effect.

Preferably, the cytokine is any cytokine or combination of cytokines that is capable of improving the growth performance of an animal. More preferably, the cytokine includes one or more of interleukin 1 (IL-1), interleukin 2 (IL-2), interleukin 3 (IL-3), interleukin 4 (IL-4), interleukin 5 (IL-5), interleukin (IL-6), interleukin 7 (IL-7), interleukin 10 (IL-10), interleukin 11 (IL-1), interleukin 12 (IL-12), interleukin 13 (IL-13), granulocyte macrophage-colony stimulating factor (GM-CSF), granulocyte colony stimulating factor (G-CSF), macrophage-colony stimulating factor (M-CSF), erythropoietin (Epo), stem cell factor (SCF), leucocyte inhibitor factor (LIF), tumour growth factor beta (TGFβ) and tumour necrosis factor alpha (TNFα).

Even more preferably, the cytokine is selected from the group consisting of interleukin 3 (IL-3), interleukin 4 (IL-4), interleukin 5 (IL-5) and granulocyte macrophage-colony stimulating factor (GM-CSF). Most preferably, the cytokine is either interleukin 3 (IL-3), interleukin 4 (IL-4) or interleukin 5 (IL-5).

In one particular embodiment, a cytokine is formulated into a composition with one or more other cytokines, pharmaceutical carriers, adjuvants or vehicles and/or antibiotics. Any known pharmaceutical carrier, adjuvant or vehicle may be used as long as it does not adversely affect the growth promoting effects of the cytokine(s).

Accordingly, in a second aspect the present invention provides a growth promoting composition comprising one or more cytokines or biologically active fragments thereof and one or more antibiotics. Preferably, the composition comprises one or more cytokines and one antibiotic. Most preferably, the composition comprises one cytokine, one antibiotic and a pharmaceutical carrier, adjuvant or vehicle.

Compositions comprising antibiotics assist in limiting the microbial load in an animal, thereby assisting the cytokine to improve growth performance in the animal. Particularly preferred antibiotics are those already in use in conventional animal production environments. However, in particular, the preferred antibiotic is selected from the group consisting of amoxycylin, penicillin, procaine, ampicillin, cloxacillin, penicillin G, benzathine, benethamine, ceftiofur, cephalonium, cefuroxime, erythromycin, tylosin, tilmicosin, oleandomycin, kitasamycin, lincomycin, spectinomycin, tetracycline, oxytetracycline, chlortetracycline, neomycin, apramycin, streptomycin, avoparcin, dimetridazole, sulfonamides (including trimethoprim and diaveridine), bacitracin, virginiamycin, monensin, salinomycin, lasalocid, narasin and olaquindox or combinations thereof. Most preferably, the antibiotic is lincomycin, spectinomycin or amoxycylin.

Depending upon the activity of the cytokine, manner of administration, age and body weight of the animal, different doses of cytokine can be used. Under certain circumstances, however, higher or lower doses may be appropriate. The administration of the dose can be carried out both by single administration in the form of an individual dose unit or else several smaller dose units and also by multiple administrations of subdivided doses at specific intervals.

It will be understood, however, that the specific dose level for any particular animal will depend upon a variety of factors including the activity of the specific cytokine employed, the age, body weight, general health, sex, diet, time of administration, and route of administration, rate of excretion and cytokine or antibiotic combination. However, generally the preferred route of administration is selected from the group consisting of oral, topical and parenteral administration.

Parenteral administration includes subcutaneous injections, aerosol, intravenous, intramuscular, intrathecal injection, infusion techniques or encapsulated cells.

The cytokines or compositions of the invention may also be administered as an additive to animal water and/or feed.

The growth performance of an animal may be determined by any know measure including increased growth rate, increased efficiency of feed use, increased final weight, increased dressed weight or decreased fat content. It will be further appreciated by those skilled in the art that the improved growth performance of an animal may result from immunoenhancement, anti-parasitic or anti-microbial effect, anti-inflammatory effect or stress reduction. More preferably, the immunoenhancement will result from a TH1/TH2 immune response, antibody isotype switching, hematopoiesis, improvement in immune function, mucosal immunity, beneficial affects on homeostatic processes such as appetite, endocrine or neural-endocrine processes.

It will be appreciated by those skilled in the art that the methods and compositions disclosed herein may be useful for any animal for which improving the growth performance is a desirable outcome. However, the present invention is particularly useful for feed animals ie those animals that are routinely farmed for meat production. Preferably, the animal is a higher artiodactyl or bird. Artiodactyls include cattle, pigs, sheep, camels, goats and horses. Birds include chickens, turkeys, geese, and ducks. More preferably, the present invention relates to animals selected from the group consisting of cattle, pigs, sheep, camels, goats, horses and chickens. Most preferably, the animals are cattle, pigs, or sheep.

In a third aspect, the cytokine is administered to an animal as a nucleic acid molecule encoding said cytokine such that upon expression of said nucleic acid molecule in the animal a growth promoting amount of cytokine is produced. Thus, the present invention provides a method for improving the growth performance of an animal comprising the step of administering to an animal in need thereof a nucleic acid molecule encoding one or more cytokines or biologically-active fragments thereof, wherein the expression of said nucleic acid molecule produces an effective growth promoting amount of one or more cytokines.

The nucleic acid molecule may be DNA, cDNA, RNA, or a hybrid molecule thereof. It will be clearly understood that the term nucleic acid molecule encompasses a full-length molecule or a biologically active fragment thereof.

Preferably the nucleic acid molecule is a DNA molecule encoding an interleukin. Most preferably, the DNA encodes interleukin 3, interleukin 4 or interleukin 5.

The nucleic acid molecule may integrate into the animal genome, or may exist as an extrachromosomal element.

The nucleic acid molecule may be administered by any known method; however, it is preferably injected subcutaneously, intravenously, or intramuscularly or administered as an aerosol.

The amount of nucleic acid that is administered will depend upon the route and site of administration as well as the particular cytokine encoded by the nucleic acid molecule. As described herein, introducing an amount of 200 μg of a nucleic acid molecule encoding a cytokine is sufficient to improve growth performance in an animal. Thus, preferably the amount of about 200 μg to 1,000 μg of a nucleic acid molecule encoding a cytokine is preferably introduced into an animal.

The nucleic acid molecule may also be delivered in a vector such as a porcine adenovirus vector. It may also be delivered as naked DNA.

Accordingly, in another aspect, the present invention provides a construct for delivering in vivo an effective amount of cytokine, comprising:

a) a nucleotide sequence encoding a cytokine or a biologically active fragment thereof;

b) a vector comprising a control sequence wherein the control sequence is capable of the controlling the expression of the nucleotide sequence of a) such that a cytokine or biologically active fragment thereof is produced which in turns improves growth performance in an animal.

Modified and variant forms of the construct may be produced in vitro, by means of chemical or enzymatic treatment, or in vivo by means of recombinant DNA technology. Such constructs may differ from those disclosed, for example, by virtue of one or more nucleotide substitutions, deletions or insertions, but substantially retain a biological activity of the construct or nucleic acid molecule of this invention.

### BRIEF DESCRIPTION OF THE FIGURES

FIG. 1 shows the percentage eosinophils of white blood cells (WBC) for individual pigs in treatment groups for 4 days prior to and 12 days after treatment with IL-5 delivered using several delivery strategies.

FIG. 2 shows the absolute white blood cell counts over time for individual pigs treated with recombinant IL-5, or IL-5 DNA delivered by IM injection or gene gun.

FIG. 3 shows the eosinophil index (statistical analysis of percentage eosinophils of WBC) comparing different delivery methods on the increase of eosinophils.

FIG. 4 shows the total group weight gain over 16 days for pigs treated with either recombinant IL-5, or pCI IL-5 delivered by various means.

FIG. 5 shows the average total weight gained per pig in each treatment group for pigs treated with either recombinant IL-5, or pCI IL-5 delivered by various means. Bars indicate group means and standard error.

FIG. 6 shows the average weight at Days 0, 7, 11 and 16 of pigs treated with either recombinant IL-5, or pCI IL-5 delivered by various means. Bars indicate group mean and standard error.

FIG. 7 shows the statistical comparison of the average percentage eosinophil of WBC over 11 days after IL-5 administration.

FIG. 8 shows the effect of different routes of IL-5 administration on percentage eosinophils of WBC.

FIG. 9 shows the eosinophil index (statistical analysis) by different routes of IL-5 administration.

FIG. 10 shows total treatment group weight of during the weaner period, for pigs treated with either IL-5 or saline in the presence or absence of in-feed antibiotic supplementation.

FIG. 11 shows the average weight per pig during the weaner period, in groups treated with either IL-5 or saline in the presence or absence of in-feed antibiotic supplementation.

FIG. 12 shows the individual weights of pigs in each group at the end of the weaner period in pigs treated with either IL-5 or saline in the presence or absence of in-feed antibiotic supplementation.

FIG. 13 shows the production loss as defined by deaths caused by infectious disease or a reduction in weight of individual pigs for groups treated with either IL-5 or saline in the presence or absence of in-feed antibiotic supplementation.

FIG. 14 shows the percentage eosinophils of WBC for individual pigs in groups treated with either IL-5 or saline in the presence or absence of in-feed antibiotic supplementation.

FIG. 15 shows a regression plot of weight gained over the weaner period versus the change in absolute eosinophil numbers for pigs treated with either saline (open dots) or IL-5 (black dots) in the presence of in-feed antibiotics.

FIG. 16 shows the average rate of gain per pig over the weaner period in groups treated with either IL-5 or saline in the presence or absence of in-feed antibiotic supplementation.

FIG. 17 shows the total treatment group weights over the weaner, grower and finisher phases for groups treated with either IL-5 or saline in the presence or absence of in-feed antibiotic supplementation.

FIG. 18 shows the average pig weight throughout production for groups treated with either IL-5 or saline in the presence or absence of in-feed antibiotic supplementation during the trial. Bars indicate group means and standard error.

FIG. 19 shows the comparison of the average weight differences between IL-5 treatment and saline treatment in the absence of antibiotics.

FIG. 20 shows the comparison of the average weight differences between IL-5 treatment and saline treatment in pigs provided with in-feed antibiotic supplementation.

FIG. 21 shows the comparison of saline treatment across the two medication levels to illustrate the effect of in-feed antibiotics on weight.

FIG. 22 shows the final weight of individual pigs treated with either saline or IL-5 in the presence or absence of antibiotic supplementation.

FIG. 23 shows the average percentage dressing in groups of pigs treated with either saline or IL-5 in the absence or presence of antibiotic supplementation. Bars indicate group means and standard error.

FIG. 24 shows the average warm carcass weight for pigs treated with either saline or IL-5 in the absence or presence of antibiotic supplementation. Bars indicate group means and standard error.

FIG. 25 shows the comparison of average weights throughout the weaner period for saline control pigs, with and without antibiotic supplements, from the 2 trials undertaken in a commercial piggery environment (Examples 4 and 5). Bars indicate group means and standard error.

FIG. 26 shows the total group weights for weaner period in pigs treated with either IL-5 or saline in the absence of in-feed antibiotics.

FIG. 27 shows the total group weights for weaner period in pigs treated with either IL-5 or saline in the presence of reduced levels of in-feed antibiotics.

FIG. 28 shows the total group weights for weaner period in pigs treated with either IL-5 or saline in the presence of normal levels of in-feed antibiotics.

FIG. 29 shows the production losses as defined by deaths caused by infectious disease or a reduction in weight of individual pigs in each group, in pigs treated with either saline or IL-5 at 3 different in-feed antibiotic supplementation levels.

FIG. 30 shows the average weights throughout the weaner period for groups of pigs treated with either IL-5 or saline in the absence of in-feed antibiotics.

FIG. 31 shows the average weights throughout the weaner period for groups of pigs treated with either IL-5 or saline and provided with reduced levels of in-feed antibiotics.

FIG. 32 shows the average weights throughout the weaner period for groups of pigs treated with either IL-5 or saline and provided with normal levels of in-feed antibiotics.

FIG. 33 shows the comparison of the average weights throughout the weaner phase for saline treated control groups provided with three different levels of feed or water antibiotic supplementation.

FIG. 34 shows the average weight gains of pigs in each group over the weaner period. Bars indicate group means and standard error.

FIG. 35 shows the average weight differences of antibiotic supplemented controls to the no-antibiotic supplemented control.

FIG. 36 shows the average difference in weight between saline controls and IL-5 treatment without antibiotics.

FIG. 37 shows the average difference in weight between saline controls and IL-5 treatment with reduced antibiotics.

FIG. 38 shows the average difference in weight between saline controls and IL-5 treatment with normal levels of antibiotics.

FIG. 39 shows the average P2 value (backfat measurement prior to slaughter) for each group. Bars indicate group means and standard error.

FIG. 40 shows a plot of P2 versus the final weight for individual pigs for IL-5 treated and controls without antibiotics.

FIG. 41 shows the average absolute eosinophil level for each group.

FIG. 42 illustrates a timeline showing sequence of events for cytokine experiment with E. coli challenge.

FIG. 43 shows the daily feed intake per pig during E. coli challenge in pigs treated with saline, Apralan or IL-5.

FIG. 44 shows E. coli cultured from faeces collected from pigs for 5 days after initial challenge with E. coli. Data points show group means with standard errors.

FIG. 45 shows the total faecal culture scores for the 5 days of E. coli challenge.

FIG. 46 shows the percentage reduction in total faecal culture scores compared to saline controls.

FIG. 47 shows the incidence of clinical signs in the form of diarrhoea and wet faeces from each group of pigs during the 5 days after E. coli challenge. Bars show the total records for each group; the maximum record for each group is 40.

FIG. 48 shows the reduction in clinical signs of diarrhoea and wet faeces in the cytokine treated animals compared to saline controls.

FIG. 49 shows E. coli culture scores for bacterial growth on sheep blood agar from samples taken in different areas along the gastro-intestinal tract at post-mortem. SI is the small intestines. Bars show group means and standard errors.

FIG. 50 shows the mean total E. coli culture scores taken from pigs at post-mortem. Bars show the group means of individual's total bacterial scores, and standard errors.

FIG. 51 shows the percentage change in total E. coli culture scores at post-mortem, compared to saline controls.

FIG. 52 shows the percentage change in E. coli culture scores obtained from the foregut and hindgut areas, compared to saline controls.

FIG. 53 shows the levels of spirochaete shedding in faeces in pigs after treatment with IL-5, Lincocin or saline and subsequent challenge with swine dysentery.

FIG. 54 shows the comparison of the number of spirochaetes cultured from the caecum, anterior colon, posterior colon and faeces at postmortem. Bars show group means and standard error.

FIG. 55 shows the reduction in the number of spirochaetes cultured from the gut at postmortem expressed as a percentage compared to saline treated controls.

FIG. 56 shows the manifestation of clinical signs of swine dysentery infection indicated by faecal condition at postmortem. Signs indicative of dysentery are faeces wet and mucoid with blood (dys) or wet and unable to hold form (wet). Bars show the incidence within the group of 8 pigs.

FIG. 57 shows the average rate of gain of groups during the weaner period (ie. treatment period).

FIG. 58 shows the comparison of the average weights of pigs in each group during the trial.

FIG. 59 shows the final average weight of pigs in each group.

FIG. 60 shows the individual weights of pigs in each group.

FIG. 61 shows the average percentage dressing of pigs in each treatment.

FIG. 62 shows the average warm carcass weight of pigs in each group at slaughter.

FIG. 63 shows the comparison of total weights of all live pigs in antibiotic treated groups.

FIG. 64 shows the comparison of the average absolute levels of eosinophils in blood of pigs administered with different doses of IL-3 to the control.

FIG. 65 shows the eosinophil index (statistical analysis) of each group.

FIG. 66 shows the basophil index (statistical analysis) of each group.

FIG. 67 shows a graph of average absolute eosinophils numbers in blood of pigs in each group.

FIG. 68 shows the percentage eosinophils of individual pigs in each treatment group.

FIG. 69 shows the average percentage eosinophils of WBC for each treatment group.

FIG. 70 shows the comparison of the average total Ig titre in sera for each group.

FIG. 71 shows the comparison of the average IgA titre in sera for each group.

FIG. 72 shows the comparison of IgG1 levels.

FIG. 73 shows the comparison of IgG2 levels.

FIG. 74 shows the average weight gain in pigs treated with recombinant cytokines, plasmid cytokines, flunix or saline during chronic challenge with App.

FIG. 75 shows the total weight gained during 14d challenge with App, in pigs treated with saline, flunix, recombinant cytokines or plasmid cytokines.

FIG. 76 shows the levels of TNFα in the serum of pigs treated with flunix, recombinant cytokines or plasmid cytokines and exposed to App challenge.

FIG. 77 shows the levels of IL-6 in peripheral blood measured by RT-PCR. Pigs treated with flunix, recombinant cytokines or plasmid cytokines and challenged with App. Data for saline-treatment was not available at 13 days after App challenge.

FIG. 78 shows the presence of clinical signs of disease between treatment groups on a per visit basis over 30 visits in the first week of challenge. The maximum score per visit was 8.

FIG. 79 shows the degree of pleurisy at necropsy, expressed as pleurisy score (0-5) in pigs treated with—saline, flunix or IL-4 and subsequently challenged with App.

FIG. 80 shows the degree of pleuropneumonia at necropsy, expressed as % affected lung by weight, in pigs treated with anti-inflammatory cytokines or flunix and challenged with App.

FIG. 81 shows the levels of spirochaete shedding in faeces in pigs after treatment with IL-4, Lincocin or saline and subsequent challenge with swine dysentery.

FIG. 82 shows the comparison of the number of spirochaetes cultured from the caecum, anterior colon, posterior colon and faeces at postmortem. Bars show group means and standard error.

FIG. 83 shows the manifestation of clinical signs of swine dysentery infection indicated by faecal condition at postmortem. Signs indicative of dysentery are faeces wet and mucoid with blood (dys) or wet and unable to hold form (wet). Bars show the incidence within the group of 8 pigs.

FIG. 84 shows the signs of gross pathology associated with infection with swine dysentery as seen in the anterior colon at postmortem. Pathology was signified as mild by the presence of patchy redness or mild colitis, or as more severe with changes in tissue or contents commonly associated with dysentery such as the presence of blood in the contents, and extensive redness and inflammation of the gut tissue.

FIG. 85 shows the signs of gross pathology associated with infection with swine dysentery as seen in the posterior colon at postmortem. Pathology was signified as mild by the presence of patchy redness or mild colitis, or as more severe with changes in tissue or contents commonly associated with dysentery such as the presence of blood in the contents, and extensive redness and inflammation of the gut tissue.

FIG. 86 shows the weekly pig weights during swine dysentery challenge. Bars indicate group means and standard error.

FIG. 87 shows the mean weight of pigs at the conclusion of the swine dysentery challenge, 19 and 20 days after infection. Bars indicate group means and standard error.

FIG. 88 shows the mean weight gain over the duration of the swine dysentery trial, from 7days prior to challenge to slaughter on days 19 and 20 after challenge. Bars indicate group means and standard errors.

### DETAILED DESCRIPTION OF THE INVENTION

The practice of the present invention employs, unless otherwise indicated, conventional molecular biology, cellular biology, and recombinant DNA techniques within the skill of the art. Such techniques are well known to the skilled worker, and are explained fully in the literature. See, e.g., Sambrook and Russell “Molecular Cloning: A Laboratory Manual” (2001) (Green Publishing, New York); Cloning: A Practical Approach,” Volumes I and II (D. N. Glover, ed., 1985) (Green Publishing, New York); “Oligonucleotide Synthesis” (M. J. Gait, ed., 1984); “Nucleic Acid Hybridisation” (B. D. Hames & S. J. Higgins, eds., 1985); “Antibodies: A Laboratory Manual” (Harlow & Lane, eds., 1988); “Transcription and Translation” (B. D. Hames & S. J. Higgins, eds., 1984); “Animal Cell Culture” (R. I. Freshney, ed., 1986); “Immobilised Cells and Enzymes” (IRL Press, 1986); B. Perbal, “A Practical Guide to Molecular Cloning” (1984), and Sambrook, et al., “Molecular Cloning: a Laboratory Manual” (1989). Ausubel, F. et al., 1989-1999, “Current Protocols in Molecular Biology” (Green Publishing, New York).

Before the present methods and compositions are described, it is understood that this invention is not limited to the particular materials and methods described, as these may vary. It is also to be understood that the terminology used herein is for the purpose of describing particular embodiments only, and is not intended to limit the scope of the present invention which will be limited only by the appended claims. It must be noted that as used herein and in the appended claims, the singular forms “a,” “an,” and “the” include plural reference unless the context clearly dictates otherwise. Thus, for example, a reference to “a cytokine” includes a plurality of such cytokines, and a reference to “an antibiotic” is a reference to one or more antibiotics and equivalents thereof known to those skilled in the art, and so forth. Unless defined otherwise, all technical and scientific terms used herein have the same meanings as commonly understood by one of ordinary skill in the art to which this invention belongs. Although any materials and methods similar or equivalent to those described herein can be used to practice or test the present invention, the preferred materials and methods are now described.

All publications mentioned herein are cited for the purpose of describing and disclosing the protocols, reagents and vectors which are reported in the publications and which might be used in connection with the invention. Nothing herein is to be construed as an admission that the invention is not entitled to antedate such disclosure by virtue of prior invention.

In describing the present invention, the following terminology is used in accordance with the definitions set out below.

### Definitions

The methods and compositions of the present invention are useful for improving the “growth performance” of an animal. The term “growth performance” is known in the art as a reference to the criteria of growth rate and efficiency of feed use of an animal, and also a reference to the final weight of an animal, and the dressed weight and fat content of a carcass from the animal. The “growth rate” of an animal is the rate of unit gain in live weight of the animal and “efficiency of feed use” is the amount of feed required per unit gain in live weight of the animal. The “final weight” of an animal is the weight of the animal at slaughter at a specified age and the “dressed weight” is the weight of a carcass from which viscera, feet, trotters or hooves have been removed. The “fat content” is the amount of fat on a dressed carcass. Methods for measuring the criteria of growth rate, efficiency of feed use, final weight, and dressed weight and fat content of a carcass, are known to the skilled worker. See, for example, Manipulating Pig Production VI, VII & VIII. 1997, 1999 & 2001, Ed. P. D. Cranwell, Australian Pig Science Association, Werribee, Victoria, Australia. Growth rate is obtained from successive measurements of live weight over time. Efficiency of feed use is obtained from successive measurements of feed disappearance and live weight over time. Carcass fat content is traditionally assumed from a measurement of back-fat thickness in millimetres at the P2 position. Accordingly, in the present invention the term “growth performance” means an improvement in one or more of the criteria of growth rate, efficiency of feed use, final or dressed weight and fat content of a carcass from an animal.

The term “animal” as used herein means any animal for which an increase in growth performance is desirable. For example, animals included in the mammalian order Artiodactyls or in the avian class Aves.

Artiodactyls comprise approximately 150 living species distributed through nine families: pigs (Suidae), peccaries (Tayassuidae), hippopotamuses (Hippopotamidae), camels (Camelidae), chevrotains (Tragulidae), giraffes and okapi (Giraffidae), deer (Cervidae), pronghorn (Antilocapridae), and cattle, sheep, goats and antelope (Bovidae). Many of these animals are used as feed animals in various countries. More importantly, with respect to the present invention, many of the economically important animals such as goats, sheep, cattle and pigs have very similar biology and share a high degree of genomic homology. More importantly, it is well known that certain animals such as goats and sheep and horses and donkeys can interbreed.

The terms “bird” and “avian” as used herein, are intended to include all avian species, including, but not limited to, chickens, turkeys, ducks, geese, quail, and pheasant which are commercially raised for eggs or meat. This term also includes both males and females of any avian species. Accordingly, the terms “bird” and “avian” are particularly intended to encompass hens, cocks and drakes of chickens, turkeys, ducks, geese, quail and pheasant. Chickens and turkeys are preferred.

All Artiodactyls have similar cytokine systems, in that they posses, for example, interleukins, GM-CSF, interferon's alpha, beta and gamma. In most species the genes coding for these cytokines map to particular regions on certain chromosomes. For example, in humans, the interleukin 5 gene maps to chromosome 5q23-31 in the same area as genes encoding GM-CSF, M-CSF, IL-3 and IL-4. More importantly, many of the cytokines have high degrees of amino acid sequence homologies between different species. For example, it is well known in the art that porcine interleukin 5 shares as much as 90% of its amino acids with animals such as bovine, ovine and equine (See, for example, Sylvin et al. (2000), Immunogenetics, 51: 59-64). Indeed, even species as distinct as mice and humans share as much as 70% amino acid sequence identities (See, for example, Dictionary of Cytokines (1995), Horst Ibelgaufts, VCH Publishers, Weinheim). Furthermore, it is known that human IL-10 has a significant degree of sequence homology with bovine, murine, and ovine IL-10 (Dutia et al. (1994) Gene; 149:393-4). Table 1 shows a list of the amino acid sequence identities of IL-3, IL-4, and IL-5 across bovine, ovine, human and murine compared to porcine.

It is also well known in the art that a number of cytokines have species cross-reactivity. For example, IL-4 has some cross-species reactivity, while IL-5 has a high level of cross-species reactivity Dictionary of Cytokines (1995), Horst Ibelgaufts, VCH Publishers, Weinheim. However, it should be noted that the cross-reactivity described in the prior art literature relates to in-vitro assays and some in-vivo experiments, but does not relate to growth performance.

Cytokines are also known to regulate the expression of cytokine receptors, either in a stimulatory or inhibitory manner, thereby controlling the biological activities of cytokines by other cytokines. Some cytokines share common receptor subunits which may have a regulatory effect.

TABLE 1
AMINO ACID SEQUENCE IDENTITIES TO PORCINE SEQUENCES

IL-3: bovine 48% ovine 47% human 39% murine 29%
IL-4: bovine 80% ovine 78% human 63% murine 42%
IL-5: bovine 90% ovine 88% human 65% murine 42% equine 83%
Identities determined from GenBank (USA) Blast searches.

For example, the GM-CSF receptor shows significant homologies with other receptors for Hematopoietic growth factors, including IL-2-beta, IL-3, IL-6, IL-7, Epo and the Prolactin receptors (See, for example, Cytokines Online Pathfinder Encyclopaedia—www.copewithcytokines.de). It is also known that IL-3 is capable of upregulating the expression of GM-CSF receptors on mouse macrophages, IL-3 also upregulates IL-1 receptor expression on human and murine bone marrow cells, IL-4 upregulates IL-1 type 1 receptor expression and down regulate IL-2 receptor expression. Furthermore, IL-7 upregulates IL-4 Receptor expression, and TNFalpha upregulates both IL-3 and GM-CSF Receptor expression (Dictionary of Cytokines (1995), Horst Ibelgaufts, VCH Publishers, Weinheim). Thus cytokines themselves could potentially be used to regulate the endogenous expression or biological activity of other cytokines.

In a similar fashion to Artiodactyls, birds also have common cytokine systems, including interleukins. Accordingly, the term “avian interleukin,” or “bird interleukin” as used herein, means any interleukin corresponding to an interleukin produced by any avian species. The term “avian” is intended to encompass various species of avian interleukin, some of which are known (See, eg., U.S. Pat. Nos. 5,028,421 and 5,106,617; M. Baggiolini and K. Clemetson, PCT Application WO 90/06321; H. Aschauer and P. Peveri, PCT Application WO 89/04836).

In brief, avian interleukins may be obtained by collecting lymphocytes from an avian donor (most conveniently from the spleen of an avian donor), growing the lymphocytes in a medium (preferably a serum-free medium) containing a T-cell mitogenic agent such as Concanavalin A, and, optionally, recovering the interleukin from the medium.

The cross-reactivity of IL-2 and IL-8 of various avian species can be routinely determined with known bioassay procedures employing IL-2 responder cells (See, eg., Gimbrone, et al., Science 246:1601, 1603 n. 14 (1989)). Those skilled in the art will be able to select an appropriate cytokine composition for the bird being treated based on the known cross-reactivities of cytokine and simple screening tests known to those skilled in the art.

Insofar as this applicant is aware, analogs of avian interleukins have not yet been synthesized. However, based on the cross-reactivity of various non-avian IL-2s, it is expected that synthetic analogs of avian interleukins, when available, can be screened for activity in the present invention in a routine manner, and should function in the present invention in substantially the same way as the naturally occurring interleukins.

Given the level of common ancestry and biology for many of the feed animals, the high degree of amino acid sequence homology for cytokines across a number of species such as cattle, sheep, goats and pigs, and the level of cross-species reactivity of these cytokines a person skilled in the art would appreciate that the compositions and methods disclosed herein are applicable for all feed animals and for all cytokines.

It will be further appreciated by those skilled in the art that the compositions and methods disclosed herein may be directly extrapolated to encompass other aspects of the invention. For example, data are presented for specific cytokines; however, these are not to be construed to be limiting on the invention. Indeed, the cytokines disclosed were specifically chosen to illustrate the breadth of the invention. For example, IL-5, IL-3 and GM-CSF are all cytokines which are capable of increasing eosinophil levels. Cytokines such as IL-4 have similar functions to IL-13 (Dictionary of Cytokines (1995), Horst Ibelgaufts, VCH Publishers, Weinheim).

Furthermore, many cytokines share receptors or receptor subunits. For example, IL-3, IL-5 and GM-CSF share a receptor subunit (Dictionary of Cytokines (1995), Horst Ibelgaufts, VCH Publishers, Weinheim). IL-4 shares a common subunit with IL-2 and IL-7 (Dictionary of Cytokines (1995), Horst Ibelgaufts, VCH Publishers, Weinheim). Some cytokines have similar gene structures and are clustered on the one chromosome eg IL-3, IL-4, IL-5, GM-CSF and IL-13 in humans and mice (Dictionary of Cytokines (1995), Horst Ibelgaufts, VCH Publishers, Weinheim).

IL-1, 3, 4, 5, 6, 11 and 12 are known hematopoietic growth factors. Similar hematopoietic growth factors include GM-CSF, G-CSF, M-CSF, Epo, stem cell factor (SCF), LIF, TGFβ and TNFα (Dictionary of Cytokines (1995), Horst Ibelgaufts, VCH Publishers, Weinheim).

IL-5 is a known late acting lineage specific factor as are Epo, M-CSF and G-CSF. Cytokines that have the same early acting multipotential ability as IL-3 and IL-4 include GM-CSF (Dictionary of Cytokines (1995), Horst Ibelgaufts, VCH Publishers, Weinheim).

A number of cytokines are regarded as TH2 cytokines (TH2; CD4+helper cells) having activity on B cells. These include IL-4, IL-5, IL-6 and IL-10. IL-3 is secreted by both TH1 and TH2 (Dictionary of Cytokines (1995), Horst Ibelgaufts, VCH Publishers, Weinheim).

IL-5 is also known in other species to upregulate circulating eosinophil cells. Furthermore, IL-5 is a potent regulator of early hematopoietic progenitor cells and stimulates the proliferation, activation and differentiation of eosinophils. The cytokine IL-3 is also known to stimulate the proliferation, activation and differentiation of eosinophils. IL-3 supports the proliferation of almost all types of hematopoietic progenitor cells. IL-3 is considered to be an early acting factor that primes hematopoietic stem cells and many of the activities of IL-3 are enhanced or depend on co-stimulation with other cytokines. Another cytokine (GM-CSF) has been reported to increase the production of eosinophils (Dictionary of Cytokines (1995), Horst Ibelgaufts, VCH Publishers, Weinheim). Like IL-3, GM-CSF supports the proliferation of many types of hematopoietic progenitor cells and primes stem cells.

In another example, it has been shown that IL-25 induces IL-4, IL-5, and IL-13 gene expression. The induction of these cytokines resulted in TH2-like responses marked by increased serum IgE, IgG(1), and IgA levels, blood eosinophilia, and pathological changes in the lungs and digestive tract that included eosinophilic infiltrates, increased mucus production, and epithelial cell hyperplasia/hypertrophy. In addition, these data showed that IL-25 induces TH2-type cytokine production by accessory cells that are MHC class II(high), CD11c(dull), and lineage(−) (See, for example, Fort MM et al (2001), Immunity, 15(6):985-95).

All of the foregoing is illustrative of the breadth of the presently disclosed invention with respect the types of animals encompassed. However, it will also be readily seen that the term “cytokine” is also to be construed broadly and not limited to the experimental data disclosed. For example, the term “cytokine” includes one or more of IL-1, IL-2, IL-3, IL-4, IL-5, IL-6, IL-7, IL-10, IL-11, IL-12, IL-13, granulocyte macrophage-colony stimulating factor (GM-CSF), granulocyte colony stimulating factor (G-CSF), macrophage-colony stimulating factor (M-CSF), erythropoietin (Epo), stem cell factor (SCF), leucocyte inhibitor factor (LIF), tumour growth factor beta (TGFβ) and tumour necrosis factor alpha (TNFα).

As used herein, the term “growth promoting amount” is meant an amount of a cytokine of the present invention effective to yield an increase in growth performance as defined above. For example, increased growth rate, efficiency of feed use, increased final weight, increased carcass dressed weight or reduced fat content.

As used herein, the term “administration” refers to the mode of delivery of a composition of the invention. The term also refers to the dosage of a composition. Depending upon the activity of a cytokine and age and body weight of an animal, the manner of administration and dosage of a cytokine will vary. It will be understood that the specific dose level for any particular animal will depend upon a variety of factors including the activity of the specific cytokine employed, the age, body weight, general health, sex, diet, time of administration, route of administration, rate of excretion and cytokine or antibiotic combination. However, generally the preferred route of administration is selected from the group consisting of oral, topical and parenteral administration.

Parenteral administration includes subcutaneous injections, aerosol, intravenous, intramuscular, intrathecal, injection or infusion techniques and encapsulated cells.

As used herein, the term “upregulate” or “upregulating” refers to inducing an increase in production, secretion or availability (and thus an increase in the concentration) of a protein or peptide. A method of upregulating endogenous interleukin in an animal or avian thus refers to a method of inducing an increase in the production, secretion or availability of cytokine in the animal or avian, as compared to an untreated animal or avian.

The term “endogenous” means originating within the subject, cell, or system being studied. Accordingly, supplementing the endogenous levels of a cytokine means that a compound or compounds is/are administered to an animal such that the total amount of a cytokine in the animal is higher than normally present. Increasing the endogenous levels of a cytokine means that a compound or compounds is/are administered to an animal where the compound or compounds increase the production of a cytokine by an animals cells or tissue, thereby effectively increasing the total amount of a cytokine in the animal. The endogenous levels of a cytokine may also be effectively increased by decreasing the turn over rate of a cytokine. For example, a compound or compounds of the invention when administered to an animal may decrease the rate of proteolysis of endogenous cytokines by inhibiting the effect of proteolytic enzymes. Furthermore, a compound or compounds may reduce the endogenous levels of a cytokine thereby providing a need to administer cytokines for effective immune responses.

Although many substances, particularly killed bacteria or bacterial extracts or non-specific mitogens of plant origin, are able to stimulate upregulation of endogenous cytokines, including IL-3, IL-4 and IL-5, they also stimulate upregulation of the pro-inflammatory cytokine mediators, which can have deleterious effects on the growth and productivity of livestock. However, extracts from some parasites, particularly helminths, have been shown to increase cytokine production (for examples, refer to Ehigiator HN et al.,(2000) Infection & Immunity. 68:4913-4922, Zang XX et al., (2000) Journal of Immunology. 165:5161-5169) For example, diet comprising substances such as fish oils, omega 3 fatty acids, vitamins E and A have been associated with reduced inflammatory responses, and thus, cytokine expression. For example:

Cannabanoids

Synthetic, low affinity ligands of cannabanoids, such as (+)-HU-211 and DMH-11C, have been shown to cause anti-inflammatory effects possibly through inhibiting the production and action of TNF-alpha and other acute phase cytokines. In addition, suppression of TNF and other cytokines such as GM-CSF, IL-6, IFN gamma, and IL-12 has also been seen following exposure to high affinity and psychoactive ligands such as marijuana and THC. However, some of these ligands have also been shown to increase rather than decrease interleukins such as IL-1, IL-4, IL-10 and IL-6, cytokines such as TNF-alpha, and chemokines such as IL-8, MIP-1, and RANTES. The endogenous ligand, anandamide, has been shown in culture to either suppress the proliferation response to prolactin or enhance the response to cytokines such as IL-3 and IL-6. This eicosanoid has also been shown to increase the production of interleukins and other cytokines. Cannabinoid receptors have been shown to be involved in some, but not all of these effects (Klein et al. (2000), Proceedings of the Society for Experimental Biology and Medicine, 225: 1-8).

Fatty Acids

n-3 polyunsaturated fatty acids (PUFA) are known to have immunomodulating effects in humans. For example, alpha-linolenic acid (ALNA), long chain n-3 PUFA, and eicosapentaenoic acid (EPA) plus docosahexanoic acid (DHA). To date most studies have examined the functions of immune cells ex vivo but there are a limited number of studies reporting in vivo measures of immune status/responses. High levels of either ALNA or EPA plus DHA decrease chemotaxis of neutrophils and monocytes, decrease production of reactive oxygen species by neutrophils and monocytes, decrease production of pro-inflammatory cytokines by monocytes and T lymphocytes, and impair T lymphocyte proliferation. Similar evidence has been found in rodents (Calder PC (1997), Nutrition Research, 21: 309-341; Calder PC (1997), Annals of Nutrition and Metabolism, 41: 203-234).

Ascorbic acid and tocopherols exert anti-inflammatory effects in studies in man and animals. In general n-6 polyunsaturated fatty acids enhance, and n-3 PUFAs and monounsaturated fatty acids suppress, cytokine mediated aspects of inflammation. In addition, n-6 PUFAs and cholesterol enhance and n-3 PUFAs suppress cytokine production (Grimble RF (1998), Nutrition Research, 18: 1297-1317).

Vitamins

The vitamin D hormone stimulates transforming growth factor TGF beta −1 and interleukin 4 (IL-4) production, which in turn may suppress inflammatory T cell activity (Deluca & Cantorna (2001), FASEB Journal, 15: 2579-2585).

Increased vitamin E in humans results in increased IL-4 production (Pallast EG et al. (1999), American Journal of Clinical Nutrition, 69:1273-1281). Mice fed the low-protein diet had lower concentrations of IL-4 and IL-5 in the small intestinal mucosa and fewer IL-4- and IL-5-containing cells in the lamina propria (P<0.05). Retinyl acetate (1 mg) significantly restored the IL-5 level and the number of IL-4- and IL-5-containing cells. After immunization with 20 μg of cholera toxin (CT), the intestinal mucosa of protein-deficient mice contained significantly less CT-specific IgA than control mice. Treatment with 1 mg of retinyl acetate prevented the decline of anti-CT IgA level in the protein-deficient mice, improving their survival rate after an exposure to 0.1 mg of CT. These results suggest that large oral supplements of vitamin A may preserve mucosal IgA level during protein malnutrition, possibly by stimulating TH2 cytokine production and thereby, inducing resistance against infection (Nikawa T et al. (1999), Journal of Nutrition, 129: 934-941). Retinoic acid (RA) can regulate isotype switching at the level of germline transcription and directs switching to IgA with the help of IL-5 and inhibits IgG1 switching (Tokuyama H & Tokuyama Y (1999), Cellular Immunology, 192: 41-47).

The term “biologically active fragment” refers to a segment of a cytokine having a biological or physiological effect in an animal that is substantially similar to the entire or complete cytokine from which it is derived. For example, a biologically active fragment of interleukin 3 may be any portion of IL-3 having greater than about 5 amino acid residues which either comprises an immune epitope or other biologically active site or wherein the portion retains IL-3 biological activity. For example, if the interleukin 3 portion retains the ability to prime hematopoietic stem cells as discussed above then this porion is a “biologically active fragment” of IL-3.

In another example, a fragment of IL-5 will need to retain one or more of the following features:

(i) Stimulate the proliferation, activation and/or differentiation of eosinophils;

(ii) Induce the proliferation and differentiation of pre-activated B cells;

(iii) Promote the generation of cytotoxic cells from thymocytes; or

(iv) Stimulate the production and secretion of IgM and IgA antibodies.

Typically, such a fragment of IL-5 is one capable of competitively inhibiting the binding of IL-5 to the IL-5 receptor.

Amino acid sequence variants of the amino acid sequence of a cytokine or biologically active fragments thereof is also encompassed. For example, where one or more amino acid residues are added at the N- or C-terminus of, or within, the cytokine sequence or its fragments as defined above. Amino acid sequence variants of a cytokine sequence or its fragments as defined above wherein one or more amino acid residues of the cytokine sequence or fragment thereof are deleted, and optionally substituted by one or more amino acid residues; and derivatives of the above cytokines or fragments thereof, wherein an amino acid residue has been covalently modified so that the resulting product is a non-naturally occurring amino acid. Again all of these variants of cytokines are encompassed by the term “biologically active fragment” as long as the cytokine variants retain the biological activity of the entire cytokine from which it derived.

As used herein, a “pharmaceutical carrier, adjuvant or vehicle” is a pharmaceutically acceptable solvent, suspending agent or vehicle for delivering the cytokine and/or antibiotic to the animal. The carrier may be liquid or solid and is selected with the planned manner of administration in mind.

The term “substantially homologous” can refer both to nucleic acid and/or amino acid sequences, means that a particular subject sequence, for example, a mutant sequence, varies from a reference sequence by one or more substitutions, deletions, or additions, the net effect of which does not result in an adverse functional dissimilarity between reference and subject sequences. For purposes of the present invention, sequences having equivalent biological activity and equivalent expression characteristics are considered substantially homologous. Sequences having lesser degrees of identity, comparable bioactivity, and equivalent expression characteristics are considered equivalents.

“Microbial” refers to recombinant proteins made in bacterial, fungal (e.g., yeast), viral (e.g. baculovirus), or plant expression systems. As a product, “recombinant microbial” defines an animal protein essentially free of native endogenous substances and unaccompanied by associated native glycosylation. Protein expressed in most bacterial cultures, e.g., E. coli, will be free of glycosylation modifications; protein expressed in yeast and insect cells will have a glycosylation pattern different from that expressed in mammalian cells.

A “nucleic acid molecule” or “polynucleic acid molecule” refers herein to deoxyribonucleic acid and ribonucleic acid in all their forms, ie. single and double-stranded DNA, cDNA, mRNA, and the like.

A “double-stranded DNA molecule” refers to the polymeric form of deoxyribonucleotides (adenine, guanine, thymine, or cytosine) in its normal, double-stranded helix. This term refers only to the primary and secondary structure of the molecule, and does not limit it to any particular tertiary forms. Thus this term includes double-stranded DNA found, inter alia, in linear DNA molecules (eg. restriction fragments), viruses, plasmids, and chromosomes. In discussing the structure of particular double-stranded DNA molecules, sequences may be described herein according to the normal convention of giving only the sequence in the 5′ to 3′ direction along the non-transcribed strand of DNA (ie. the strand having a sequence homologous to the mRNA).

A DNA sequence “corresponds” to an amino acid sequence if translation of the DNA sequence in accordance with the genetic code yields the amino acid sequence (ie. the DNA sequence “encodes” the amino acid sequence).

One DNA sequence “corresponds” to another DNA sequence if the two sequences encode the same amino acid sequence.

Two DNA sequences are “substantially similar” when at least about 85%, preferably at least about 90%, and most preferably at least about 95%, of the nucleotides match over the defined length of the DNA sequences. Sequences that are substantially similar can be identified in a Southern hybridization experiment, for example under stringent conditions as defined for that particular system. Defining appropriate hybridization conditions is within the skill of the art. See eg. Sambrook et al., DNA Cloning, vols. I, II and III. Nucleic Acid Hybridization. However, ordinarily, “stringent conditions” for hybridization or annealing of nucleic acid molecules are those that

(1) employ low ionic strength and high temperature for washing, for example, 0.015M NaCl/0.0015M sodium citrate/0.1% sodium dodecyl sulfate (SDS) at 500C, or

(2) employ during hybridization a denaturing agent such as formamide, for example, 50% (vol/vol) formamide with 0.1% bovine serum albumin/0.1% Ficoll/0.1% polyvinylpyrrolidone/50 mM sodium phosphate buffer at pH 6.5 with 750 mM NaCl, 75 mM sodium citrate at 42° C.

Another example is use of 50% formamide, 5×SSC (0.75M NaCl, 0.075M sodium citrate), 50 mM sodium phosphate (pH 6.8), 0.1% sodium pyrophosphate, 5×Denhardt's solution, sonicated salmon sperm DNA (50 μg/mL), 0.1% SDS, and 10% dextran sulfate at 42° C., with washes at 42° C. in 0.2×SSC and 0.1% SDS.

A “heterologous” region or domain of a DNA construct is an identifiable segment of DNA within a larger DNA molecule that is not found in association with the larger molecule in nature. Thus, when the heterologous region encodes a mammalian gene, the gene will usually be flanked by DNA that does not flank the mammalian genomic DNA in the genome of the source organism. Another example of a heterologous region is a construct where the coding sequence itself is not found in nature (eg. a cDNA where the genomic coding sequence contains introns or synthetic sequences having codons different than the native gene). Allelic variations or naturally-occurring mutational events do not give rise to a heterologous region of DNA as defined herein.

A “coding sequence” is an in-frame sequence of codons that correspond to or encode a protein or peptide sequence. Two coding sequences correspond to each other if the sequences or their complementary sequences encode the same amino acid sequences. A coding sequence in association with appropriate regulatory sequences may be transcribed and translated into a polypeptide in vivo. A polyadenylation signal and transcription termination sequence will usually be located 3′ to the coding sequence.

A “promoter sequence” is a DNA regulatory region capable of binding RNA polymerase in a cell and initiating transcription of a downstream (3′ direction) coding sequence. A coding sequence is “under the control” of the promoter sequence in a cell when RNA polymerase which binds the promoter sequence transcribes the coding sequence into mRNA, which is then in turn translated into the protein encoded by the coding sequence.

For the purposes of the present invention, the promoter sequence is bounded at its 3′ terminus by the translation start codon of a coding sequence, and extends upstream to include the minimum number of bases or elements necessary to initiate transcription at levels detectable above background. Within the promoter sequence will be found a transcription initiation site (conveniently defined by mapping with nuclease S1), as well as protein binding domains (consensus sequences) responsible for the binding of RNA polymerase. Eukaryotic promoters will often, but not always, contain “TATA” boxes and “CAT” boxes; prokaryotic promoters contain Shine-Delgarno sequences in addition to the −10 and −35 consensus sequences.

A cell has been “transformed” by exogenous DNA when such exogenous DNA has been introduced inside the cell wall. Exogenous DNA may or may not be integrated (covalently linked) to chromosomal DNA making up the genome of the cell. In prokaryotes and yeast, for example, the exogenous DNA may be maintained on an episomal element such as a plasmid. With respect to eukaryotic cells, a stably transformed cell is one in which the exogenous DNA is inherited by daughter cells through chromosome replication. This stability is demonstrated by the ability of the eukaryotic cell to establish cell lines or clones comprised of a population of daughter cells containing the exogenous DNA. “Integration” of the DNA may be effected using non-homologous recombination following mass transfer of DNA into the cells using microinjection, biolistics, electroporation or lipofection. Alternative methods such as homologous recombination, and or restriction enzyme mediated integration (REMI) or transposons are also encompassed, and may be considered to be improved integration methods.

A “clone” is a population of cells derived from a single cell or common ancestor by mitosis. “Cell,” “host cell,” “cell line,” and “cell culture” are used interchangeably herewith and all such terms should be understood to include progeny. A “cell line” is a clone of a primary cell that is capable of stable growth in vitro for many generations. Thus the words “transformants” and “transformed cells” include the primary subject cell and cultures derived therefrom, without regard for the number of times the cultures have been passaged. It should also be understood that all progeny might not be precisely identical in DNA content, due to deliberate or inadvertent mutations.

Vectors are used to introduce a foreign substance, such as DNA, RNA or protein, into an organism. Typical vectors include recombinant viruses (for DNA) and liposomes (for protein). A “DNA cloning vector” is an autonomously replicating DNA molecule, such as plasmid, phage or cosmid. Typically the DNA cloning vector comprises one or a small number of restriction endonuclease recognition sites, at which such DNA sequences may be cut in a determinable fashion without loss of an essential biological function of the vector, and into which a DNA fragment may be spliced in order to bring about its replication and cloning. The cloning vector may also comprise a marker suitable for use in the identification of cells transformed with the cloning vector.

An “expression vector” is similar to a DNA cloning vector, but contains regulatory sequences which are able to direct protein synthesis by an appropriate host cell. This usually means a promoter to bind RNA polymerase and initiate transcription of mRNA, as well as ribosome binding sites and initiation signals to direct translation of the mRNA into a polypeptide. Incorporation of a DNA sequence into an expression vector at the proper site and in correct reading frame, followed by transformation of an appropriate host cell by the vector, enables the production of mRNA corresponding to the DNA sequence, and usually of a protein encoded by the DNA sequence.

For the purposes of the present invention, the promoter sequence is bounded at its 3′ terminus by the translation start codon of a coding sequence, and extends upstream to include the minimum number of bases or elements necessary to initiate transcription at levels detectable above background. Within the promoter sequence will be found a transcription initiation site (conveniently defined by mapping with nuclease S1), as well as protein binding domains (consensus sequences) responsible for the binding of RNA polymerase.

An “exogenous” element is one that is foreign to the host cell, or is homologous to the host cell but in a position within the host cell in which the element is ordinarily not found.

“Digestion” of DNA refers to the catalytic cleavage of DNA with an enzyme that acts only at certain locations in the DNA. Such enzymes are called restriction enzymes or restriction endonucleases, and the sites within DNA where such enzymes cleave are called restriction sites. If there are multiple restriction sites within the DNA, digestion will produce two or more linearized DNA fragments (restriction fragments). The various restriction enzymes used herein are commercially available, and their reaction conditions, cofactors, and other requirements as established by the enzyme manufacturers are used. Restriction enzymes are commonly designated by abbreviations composed of a capital letter followed by other letters representing the microorganism from which each restriction enzyme originally was obtained and then a number designating the particular enzyme. In general, about 1 μg of DNA is digested with about 1-2 units of enzyme in about 20 μl of buffer solution. Appropriate buffers and substrate amounts for particular restriction enzymes are specified by the manufacturer, and/or are well known in the art.

“Recovery” or “isolation” of a given fragment of DNA from a restriction digest typically is accomplished by separating the digestion products, which are referred to as “restriction fragments,” on a polyacrylamide or agarose gel by electrophoresis, identifying the fragment of interest on the basis of its mobility relative to that of marker DNA fragments of known molecular weight, excising the portion of the gel that contains the desired fragment, and separating the DNA from the gel, for example by electroelution.

“Ligation” refers to the process of forming phosphodiester bonds between two double-stranded DNA fragments. Unless otherwise specified, ligation is accomplished using known buffers and conditions with 10 units of T4 DNA ligase per 0.5 μg of approximately equimolar amounts of the DNA fragments to be ligated.

“oligonucleotides” are short-length, single- or double-stranded polydeoxynucleotides that are chemically synthesized by known methods (involving, for example, triester, phosphoramidite, or phosphonate chemistry), such as described by Engels, et al., Agnew. Chem. Int. Ed. Engl. 28:716-734 (1989). They are then purified, for example, by polyacrylamide gel electrophoresis.

“Polymerase chain reaction,” or “PCR,” as used herein generally refers to a method for amplification of a desired nucleotide sequence in vitro, as described in U.S. Pat. No. 4,683,195. In general, the PCR method involves repeated cycles of primer extension synthesis, using two oligonucleotide primers capable of hybridizing preferentially to a template nucleic acid. Typically, the primers used in the PCR method will be complementary to nucleotide sequences within the template at both ends of or flanking the nucleotide sequence to be amplified, although primers complementary to the nucleotide sequence to be amplified also may be used. Wang, et al., in PCR Protocols, pp.70-75 (Academic Press, 1990); Ochman, et al., in PCR Protocols, pp. 219-227; Triglia, et al., Nucl. Acids Res. 16:8186 (1988).

“PCR cloning” refers to the use of the PCR method to amplify a specific desired nucleotide sequence that is present amongst the nucleic acids from a suitable cell or tissue source, including total genomic DNA and cDNA transcribed from total cellular RNA. Frohman, et al., Proc. Nat. Acad. Sci. USA 85:8998-9002 (1988); Saiki, et al., Science 239:487-492 (1988); Mullis, et al., Meth. Enzymol. 155:335-350 (1987).

A “vector” or “construct” refers to a plasmid or virus or genomic integration comprising a transcriptional unit with (1) a genetic element or elements having a regulatory role in gene expression, for example, promoters or enhancers, (2) a structural or coding sequence which is transcribed into mRNA and translated into protein, and (3) appropriate transcription initiation and termination sequences. Structural units intended for use in yeast or eukaryotic expression systems would include a leader sequence enabling extracellular secretion of translated protein by a host cell. Alternatively, where recombinant protein is expressed without a leader or transport sequence, it may include an N-terminal methionine residue. This residue may or may not be subsequently cleaved from the expressed recombinant protein to provide a final product. Generally, recombinant expression vectors will include origins of replication and selectable markers permitting transformation of the host cell, and a promoter derived from a highly-expressed gene to induce transcription of a downstream structural sequence. The heterologous structural sequence is assembled in appropriate phase with translation initiation and termination sequences, and preferably, a leader sequence capable of directing secretion of translated protein into the periplasmic space or extracellular medium. Optionally, the heterologous sequence can encode a fusion protein including an N-terminal identification peptide imparting desired characteristics, e.g., stabilization or simplified purification of expressed recombinant product. Preferred recombinant expression vectors of the invention are viral vectors (eg. porcine adenoviral vector, mammalian cells (eg. porcine cells), plant cells and bacterial cells.

The term “immune response” is meant to refer to any response to an antigen or antigenic determinant by the immune system of a vertebrate subject. Exemplary immune responses include humoral immune responses (e.g. production of antigen-specific antibodies) and cell-mediated immune responses (e.g. lymphocyte proliferation), as defined herein below.

The term “systemic immune response” is meant to refer to an immune response in the lymph node-, spleen-, or gut-associated lymphoid tissues wherein cells, such as B lymphocytes, of the immune system are developed. For example, a systemic immune response can comprise the production of serum IgG's. Further, systemic immune response refers to antigen-specific antibodies circulating in the blood stream and antigen-specific cells in lymphoid tissue in systemic compartments such as the spleen and lymph nodes. In contrast, the gut-associated lymphoid tissue (GALT) is a component of the mucosal immune system since antigen-specific cells that respond to gut antigens/pathogens are induced and detectable in the GALT.

### Preferred Embodiments

In one particularly preferred embodiment, the present invention provides a method of increasing growth performance comprising the step of administrating to an animal in need thereof a growth promoting amount of one or more cytokines or biologically active fragments thereof.

As cytokines are endogenously expressed in all feed animal species and that many of these have a high degree of cross-reactivity, it follows that cytokines from one species may be administered to animals of a different species and vice versa. For example, when the animal is a pig, human cytokines such as IL-5 may be used in the disclosed methods. There is no requirement that the particular cytokine is identical to the cytokine which is endogenously expressed in the animal.

The purpose of administering a cytokine to an animal is to improve its growth performance. The improvement of growth performance is observed in animals administered with one or more cytokines or one or more cytokines together with one or more antibiotics as compared with animals administered with antibiotic only. As discussed elsewhere growth performance is measurable; however, why there is an increase in growth performance is a little more complex. While not wishing to be bound by any particular theory or hypothesis, the applicant believes that the administration of cytokines acts in a number of complementary ways that result in the improved growth performance. For example, the applicant has found that by improving the immunity of feed animals, stock losses are avoided and consequently growth performance improves. Thus, the present invention provides a method of reducing the susceptibility of an animal to infection. The method is useful for reducing susceptibility to infection by bacteria, virus or parasite.

The applicant has also found that the administration of one or more cytokines together with one or more antibiotics also improves the growth performance of an animal while reducing the total amount of antibiotic used. It is believed that antibiotic limits the microbial load in the animal to a threshold level at which the administered cytokine is then capable of exerting an effect on growth performance.

Accordingly, the applicant believes that rather than functioning as a growth promoter per se, although this may be possible, it will be understood that administration of the cytokines may cause improved growth performance by activating the humoral and cellular arms of the immune response which are capable of being activated by the cytokines. For example, IL-5 induces eosinophil differentiation, proliferation and activation; IgA secretion, thereby decreasing the microbial load on the animal which would otherwise limit growth performance of the animal. Specifically, as described herein, no deaths were observed in a group of animals which were administered with IL-5 and antibiotic and maintained in a ‘commercial’ husbandry environment, and the animals of this group were of generally improved health and condition as compared with animals in other groups not receiving IL-5 and antibiotic.

The methods of this invention involve in one embodiment:

(1) The administration of one or more cytokines, prior to, together with, or subsequent to the administration of one or more antibiotics; or

(2) The administration of a composition comprising one or more cytokines and one or more antibiotics.

(3) The administration of one or more cytokines without any antibiotics.

The cytokine(s) or composition(s) of the invention may be administered orally, topically, or parenterally in dosage unit formulations containing conventional non-toxic pharmaceutically acceptable carriers, adjuvants, and vehicles. The term parenteral as used herein includes subcutaneous injections, aerosol, intravenous, intramuscular, intrathecal, intracranial, injection or infusion techniques.

The present invention also provides suitable topical, oral, and parenteral pharmaceutical formulations for use in the novel methods of improving growth performance of the present invention. The compositions of the present invention may be administered orally as tablets, aqueous or oily suspensions, lozenges, troches, powders, granules, emulsions, capsules, syrups or elixirs. The composition for oral use may contain one or more agents selected from the group of sweetening agents, flavouring agents, colouring agents and preserving agents in order to produce pharmaceutically elegant and palatable preparations. The tablets contain the active ingredient in admixture with non-toxic pharmaceutically acceptable carriers, adjuvants or vehicles which are suitable for the manufacture of tablets.

These carriers, adjuvants or vehicles may be, for example, (1) inert diluents, such as calcium carbonate, lactose, calcium phosphate or sodium phosphate; (2) granulating and disintegrating agents, such as corn starch or alginic acid; (3) binding agents, such as starch, gelatine or acacia; and (4) lubricating agents, such as magnesium stearate, stearic acid or talc. These tablets may be uncoated or coated by known techniques to delay disintegration and absorption in the gastrointestinal tract and thereby provide a sustained action over a longer period. For example, a time delay material such as glyceryl monostearate or glyceryl distearate may be employed. Coating may also be performed using techniques described in the U.S. Pat. Nos. 4,256,108; 4,160,452; and 4,265,874 to form osmotic therapeutic tablets for control release.

The cytokines as well as the antibiotics useful in the methods of the invention can be administered, for in vivo application, parenterally by injection or by gradual perfusion over time independently or together. Administration may be intravenously, intra-arterial, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's intravenous vehicles include fluid and nutrient replenishers, electrolyte replenishers (such as those based on Ringer's dextrose), and the like. Preservatives and other additives may also be present such as, for example, anti-microbials, anti-oxidants, chelating agents, growth factors and inert gases and the like.

The invention includes various compositions useful for improving growth performance. The compositions according to one embodiment of the invention are prepared by bringing one or more cytokines or biologically active fragments thereof, with or without one or more antibiotics into a form suitable for administration to an animal using carriers, adjuvants, vehicles or additives.

Antibiotics suitable for use in this aspect of the invention are those conventionally used in animal husbandry as an additive to animal water and/or feed and for limiting microbial load in the animal. Examples of these antibiotics include lincomycin, spectinomycin and amoxycillin. A detailed analysis of antibiotic usage for food-producing animals in Australia is described in “The use of antibiotics in food-producing animals: antibiotic-resistant bacteria in animals and humans”. Report of the Joint Expert Advisory Committee on Antibiotic Resistance (JETACAR), Commonwealth of Australia, 1999.

An antibiotic can be administered to the animal in an amount that is the same as the amount which would be conventionally administered to the animal for the purpose of decreasing microbial load in the animal. These amounts of antibiotic are known to the skilled worker and referred to in JETACAR above.

Frequently used carriers, adjuvants or vehicles include magnesium carbonate, titanium dioxide, lactose, mannitol and other sugars, talc, milk protein, gelatine, starch, vitamins, cellulose and its derivatives, animal and vegetable oils, polyethylene glycols and solvents, such as sterile water, alcohols, glycerol and polyhydric alcohols. Intravenous vehicles include fluid and nutrient replenishers. Preservatives include antimicrobial, anti-oxidants, chelating agents and inert gases. Other pharmaceutically acceptable carriers include aqueous solutions, non-toxic excipients, including salts, preservatives, buffers and the like, as described, for instance, in Remington's Pharmaceutical Sciences, 15th ed. Easton: Mack Publishing Co., 1405-1412,1461-1487 (1975) and The National Formulary XIV., 14th ed. Washington: American Pharmaceutical Association (1975), the contents of which are hereby incorporated by reference. The pH and exact concentration of the various components of the pharmaceutical composition are adjusted according to routine skills in the art. See Goodman and Gilman's The Pharmacological Basis for Therapeutics (7th ed.).

The pharmaceutical compositions according to the invention may be administered locally or systemically in a growth promoting amount. Amounts effective for this use will, of course, depend on the cytokine and the weight and general state of the animal. Typically, dosages used in vitro may provide useful guidance in the amounts useful for in situ administration of the compositions. Various considerations are described, eg., in Langer, Science, 249: 1527, (1990). Formulations for oral use may be in the form of hard gelatine capsules wherein the active ingredient is mixed with an inert solid diluent, for example, calcium carbonate, calcium phosphate or kaolin. They may also be in the form of soft gelatine capsules wherein the active ingredient is mixed with water or an oil medium, such as peanut oil, liquid paraffin or olive oil.

Aqueous suspensions normally contain the active materials in admixture with excipients suitable for the manufacture of aqueous suspension. Such excipients may be (1) suspending agent such as sodium carboxymethyl cellulose, methyl cellulose, hydroxypropylmethylcellulose, sodium alginate, polyvinylpyrrolidone, gum tragacanth and gum acacia; (2) dispersing or wetting agents which may be (a) naturally occurring phosphatide such as lecithin; (b) a condensation product of an alkylene oxide with a fatty acid, for example, polyoxyethylene stearate; (c) a condensation product of ethylene oxide with a long chain aliphatic alcohol, for example, heptadecaethylenoxycetanol; (d) a condensation product of ethylene oxide with a partial ester derived from a fatty acid and hexitol such as polyoxyethylene sorbitol monooleate, or (e) a condensation product of ethylene oxide with a partial ester derived from fatty acids and hexitol anhydrides, for example polyoxyethylene sorbitan monooleate.

The compositions may be in the form of a sterile injectable aqueous or oleagenous suspension. This suspension may be formulated according to known methods using those suitable dispersing or wetting agents and suspending agents which have been mentioned above. The sterile injectable preparation may also a sterile injectable solution or suspension in a non-toxic parenterally-acceptable diluent or solvent, for example, as a solution in 1,3-butanediol. Among the acceptable vehicles and solvents that may be employed are water, Ringer's solution, and isotonic sodium chloride solution. In addition, sterile, fixed oils are conventionally employed as a solvent or suspending medium. For this purpose, any bland fixed oil may be employed including synthetic mono-or diglycerides. In addition, fatty acids such as oleic acid find use in the preparation of injectables.

Cytokines and compositions of the invention may also be administered in the form of liposome delivery systems, such as small unilamellar vesicles, large unilamellar vesicles, and multilamellar vesicles. Liposomes can be formed from a variety of phospholipids, such as cholesterol, stearylamine, or phosphatidylcholines.

Dosage levels of the cytokines or compositions of the present invention are of the order of about 1 microgram to about 50 microgram per kilogram body weight, with a preferred dosage range between about 5 microgram to about 20 microgram per kilogram body weight per—dose (could be multiple or single)(from about 100 micrograms to about 500 micrograms per animal per dose). The amount of cytokine that may be combined with the carrier materials to produce a single dosage will vary depending upon the animal and the particular mode of administration. For example, a formulation intended for intravenous administration to a pig may contain about 20 μg to 1 g of cytokine with an appropriate and convenient amount of carrier material which may vary from about 5 to 95 percent of the total composition. Dosage unit forms will generally contain between from about 5 μg to 500 mg of cytokine.

It will be understood, however, that the specific dose level for any particular animal will depend upon a variety of factors including the activity of the specific cytokine employed, the age, body weight, general health, diet, time of administration, route of administration, rate of excretion and drug combination.

In one particularly preferred embodiment of the present invention the cytokine or cytokines are expressed in vivo rather than administered exogenously. For example, by inserting a structural DNA sequence encoding a cytokine together with suitable translation initiation and termination signals in operable reading phase with a functional promoter an expression vector is created which would be able to express the cytokine in vivo. The vector will comprise one or more phenotypic selectable markers and an origin of replication to ensure amplification within the host. Suitable prokaryotic hosts for transformation include E. coli, Bacillus subtilis, Salmonella typhimurium and various species within the genera Pseudomonas, Streptomonas; and Staphylococcus, although others may also be employed as a matter of choice. Following transformation of a suitable host strain and expression, the cells are cultured for an additional period. Cells are typically harvested by centrifugation, disrupted by physical or chemical means, and the resulting crude extract retained for further purification. Various mammalian cell culture systems can also be employed to express recombinant protein. Examples of mammalian expression systems include the COS-7 lines of monkey kidney fibroblasts, described by Gluzman, Cell 23:175 (1981), and other cell lines capable of expressing a compatible vector, for example, the C127, 3T3, CHO, HeLa and BHK cell lines and of course porcine cells. Mammalian expression vectors will comprise an origin of replication, a suitable promoter, and enhancer, and also any necessary ribosome binding sites, polyadenylation sites, splice donor and acceptor sites, transcriptional termination sequences, and 5′ flanking non-transcribed sequences. DNA sequences derived from the SV40 viral genome, for example, SV40 origin, early promoter, enhancer, splice, and polyadenylation sites may be used to provide the required non-transcribed genetic elements. Recombinant protein produced in bacterial culture is usually isolated by initial extraction from cell pellets, followed by one or more salting out, aqueous ion exchange or size exclusion chromatography steps. Protein refolding steps can be used, as necessary, in completing configuration of the mature protein. Finally, high performance liquid chromatography (HPLC) can be employed for final purification steps. Microbial cells employed in expression of proteins can be disrupted by any convenient method, including freeze-thaw cycling, sonication, mechanical disruption, or use of cell lysing agents. Use of an expression system that expresses a tag sequence for purification would simplify purification. Recombinant expression systems as defined herein will express heterologous protein upon induction of the regulatory elements linked to the DNA segment or synthetic gene to be expressed. Cell-free translation systems can also be employed to produce porcine cytokines using RNAs derived from the DNA constructs of the present invention. Appropriate cloning and expression vectors for use with prokaryotic and eukaryotic hosts are described by Maniatis, Molecular Cloning: A Laboratory Manual, (Cold Spring Harbor, N.Y., 1985), the disclosure of which is hereby incorporated by reference.

The nucleic acid encoding a particular cytokine is advantageously in the form of plasmid DNA or a viral vector (which vector is derived from an adenovirus, retrovirus, poxvirus, in particular from a vaccinia virus or an MVA virus, herpes virus, adenovirus-associated virus, etc.). The nucleic acid encoding a particular cytokine is transported by means of an infectious viral particle or in the form of a synthetic vector (cationic lipid, liposome, cationic polymer, etc.) or an engineered cell (cell which is transfected or transduced with the said nucleic acid) or non-engineered cell (which naturally contains the said nucleic acid).

According to an additionally preferred variant, the nucleic acid of interest is carried by an adenoviral vector which is defective for replication (unable to replicate autonomously in a host cell). The technology of adenoviruses is described in the state of the art (see, for example, Graham and Prevec in Methods in Molecular Biology, 1991, vol 7, pp. 109-128, ed E. J. Murey, The Human Press Inc). Advantageously, the adenoviral vector which is used within the context of the present invention is derived from the genome of an adenovirus, comprises at least the ITRs (inverted terminal repeats) and an encapsidation sequence and lacks all or part of the E1 adenoviral region. In addition, it can lack all or part of the E3 adenoviral region. However, according to an advantageous embodiment, preference is given to retaining the part of the E3 region which encodes polypeptides, in particular the glycoprotein gp19 k (Gooding et al., Critical Review of Immunology, 1990, 10: 53-71), which make it possible to escape the immune system of the host. Furthermore, the vector can contain additional deletions or mutations which affect, in particular, all or part of one or more regions selected from the E2, E4, L1, L2, L3, L4 and L5 regions (see, for example, international application WO 94/28152). In order to illustrate this point, mention may be made of the temperature-sensitive mutation which affects the DBP (standing for DNA-binding protein) gene of the E2 A region (Ensinger et al., J. Virol., 1972, 10: 328-339). Another variant, or attractive combination, consists in deleting the E4 region with the exception of the sequences which encode open reading frames (ORFS) 6 and 7 (these limited deletions do not require the E4 function to be complemented; Ketner et al., Nucleic Acids Res., 1989, 17: 3037-3048). Preferably, the gene(s) of interest is/are inserted into the vector in place of the deleted adenoviral regions, in particular the E1 region. When several genes of interest are used, they can be inserted at the same site or at different sites in the viral genome and can be under the control of the same regulatory elements or of independent elements and, where appropriate, some of them can be in the opposite orientation to the others in order to minimize the phenomena of interference at the level of their expression. The genome of the recombinant adenoviral vector can be prepared by molecular biology techniques or by homologous recombination (see WO 96/17070).

The adenoviral vectors which are used within the context of the present invention are propagated in a complementing cell line which is able to supply the defective function(s) in trans in order to produce the peptides which are required for forming the infectious viral particles. For example, use will be made of cell line 293 for complementing the E1 function (Graham et al., J. Gen. Virol., 1977, 36: 59-72) or of the cell lines described in international application WO 97/04119 for effecting a double complementation. It is also possible to employ an appropriate cell line and a helper virus in order to complement all the defective functions. The viral particles which are produced are recovered from the cell culture and, if need be, purified using the techniques of the art (caesium chloride gradient, chromatographic steps, etc.).

The adenoviral vector which is used within the context of the present invention can be derived from the genome of an adenovirus of human, canine, avian, bovine, murine, ovine, porcine or simian origin or else from a hybrid which comprises adenoviral genome fragments of different origins. Mention may be made, more specifically, of the CAV-1 or CAV-2 adenoviruses of canine origin, of DAV of avian origin, or else of type 3 Bad of bovine origin (Zakharchuk et al., Arch. Virol., 1993, 128: 171-176; Spibey and Cavanagh, J. Gen. Virol., 1989, 70: 165-172; Jouvenne et al., Gene, 1987, 60: 21-28; Mittal et al., J. Gen. Virol., 1995, 76: 93-102). However, preference will be given to an adenoviral vector that is specific for the particular animal species being studies. For example, porcine adenovirus (PAV) would be administered to pigs.

Throughout the specification, the word “comprise” and variations of the word, such as “comprising” and “comprises”, means “including but not limited to” and is not intended to exclude other additives, components, integers or steps.

The invention will now be further described by way of reference only to the following non-limiting examples. It should be understood, however, that the examples following are illustrative only, and should not be taken in any way as a restriction on the generality of the invention described above. For example, while the majority of the examples relate to pigs, it is to be understood that the invention can also be applied to other animals as disclosed herein, including for example, sheep, cattle and chickens.

### EXAMPLE 1

### Effect on Circulating Eosinophils Levels in Pigs Administered IL-5

This trial compared the effects of recombinant porcine IL-5 protein and DNA delivered porcine IL-5 on eosinophil numbers in the blood of pigs.

Experimental Design

| Treatments | Administered |
|---|---|
| 1. 100 μg IL-5 | injected IM, hind leg on 2 |
| | consecutive days (recIL-5 × 2) |
| 2. 100 μg IL-5 | injected IM, hind leg on 1 day |
| | (recIL-5 × 1) |
| 3. 200 μg pCI-IL5 DNA | needle IM, hind leg |
| 4. 10 μg pCI-IL5 | genegun, on belly and hind legs |
| 5. 200 μg PCI control | needle IM, hind leg |

Note:
Genegun: DNA coated on gold particles
IM: Intramuscular

6 pigs per treatment, approximately 7-8 weeks of age (weaners), mean weight of approx. 15 kg.

The experiment was conducted using medicated feed Barastoc EziWean 150 then Bunge Grolean CREEP ad libitum in an experimental environment (PC2 containment facilities).

Recombinant IL-5 was expressed in E. coli and purified using a GST tag system. (See, for example, Smith, D. B and Johnson, K. S. (1988), Gene, 67:31-40). IL-5 was cleaved from the GST tag, purified and tested in bioassays to confirm activity.

IL-5 cDNA (including the signal sequence) was cloned into the pCI DNA vector. DNA was purified using the Qiagen Giga Prep kit (Qiagen Inc. USA).

Total white cells and eosinophils were counted from slides.

| | |
|---|---|
| Day-4 | Bleed for haematology |
| Day-3 | Bleed for haematology |
| Day 0 | Weigh and group to standardise mean weights, |

Protocol Undertaken

Pre-bleed for haematology and dose with all treatments (1, 2, 3, 4, 5 as shown above)

8 hrs later, bleed for haematology

| | |
|---|---|
| Day 1 | Re-dose treatment 1, bleed for haematology |
| Day 2 | Bleed for haematology |
| Day 3 | Bleed for haematology |
| Day 4 | Bleed for haematology |
| Day 7 | Bleed for haematology, weigh |
| Day 9 | Bleed for haematology |
| Day 11 | Bleed for haematology, weigh |
| Day 16 | weigh. |

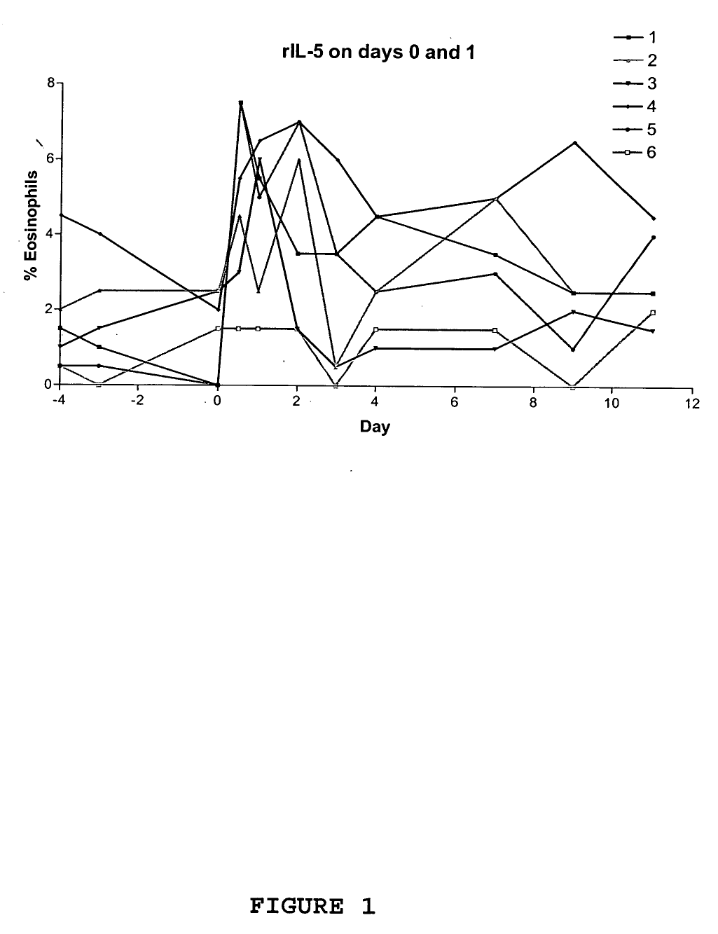

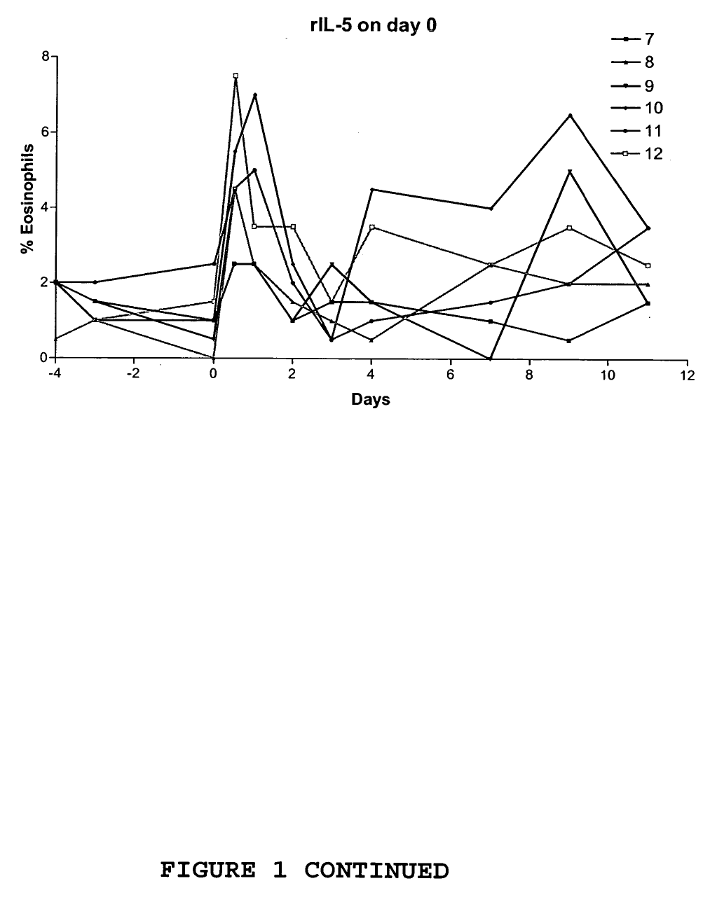

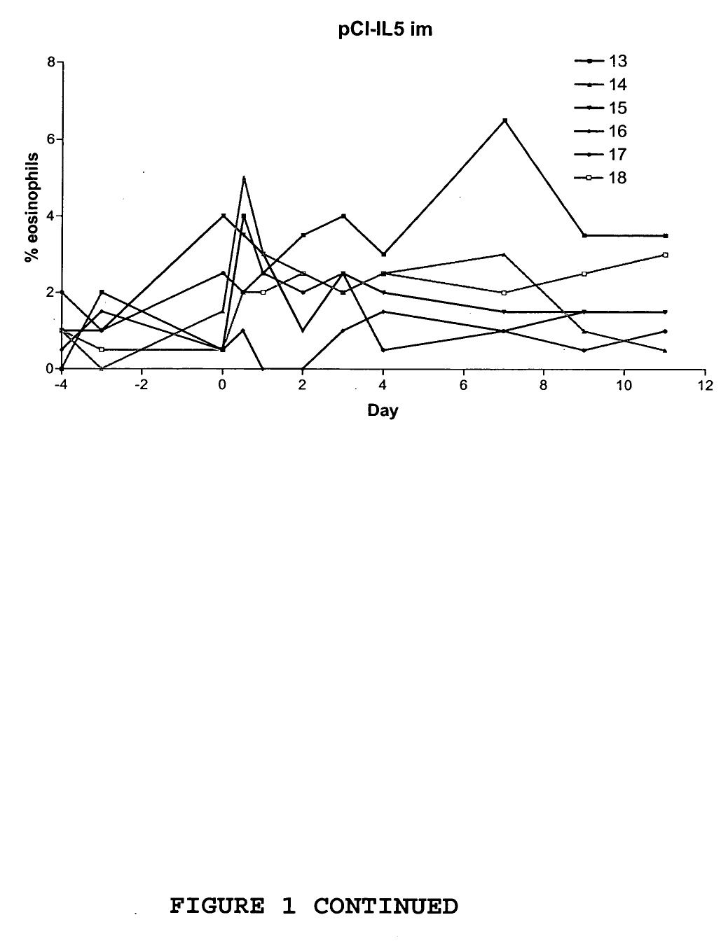

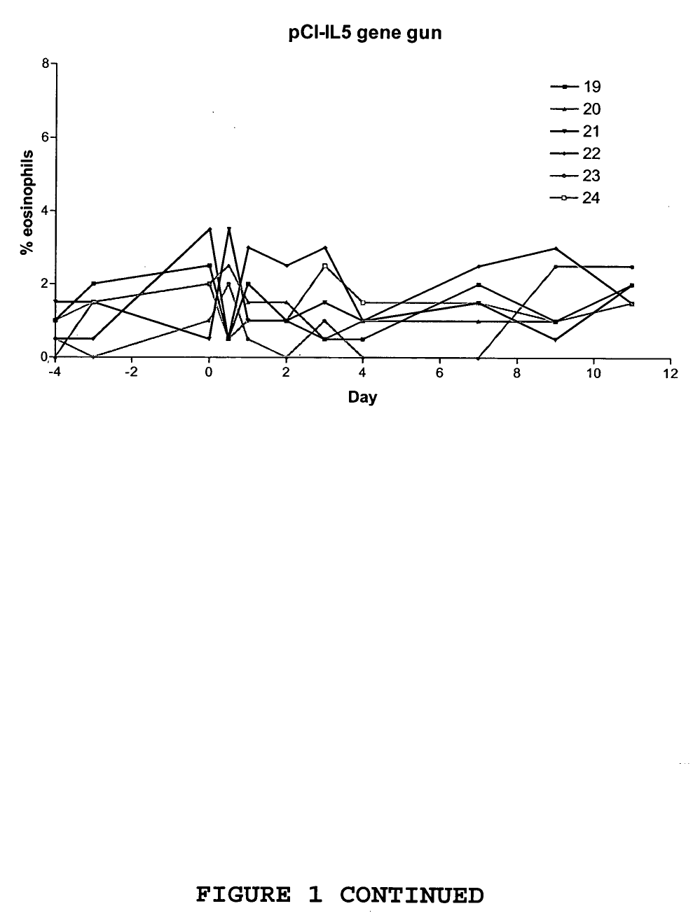

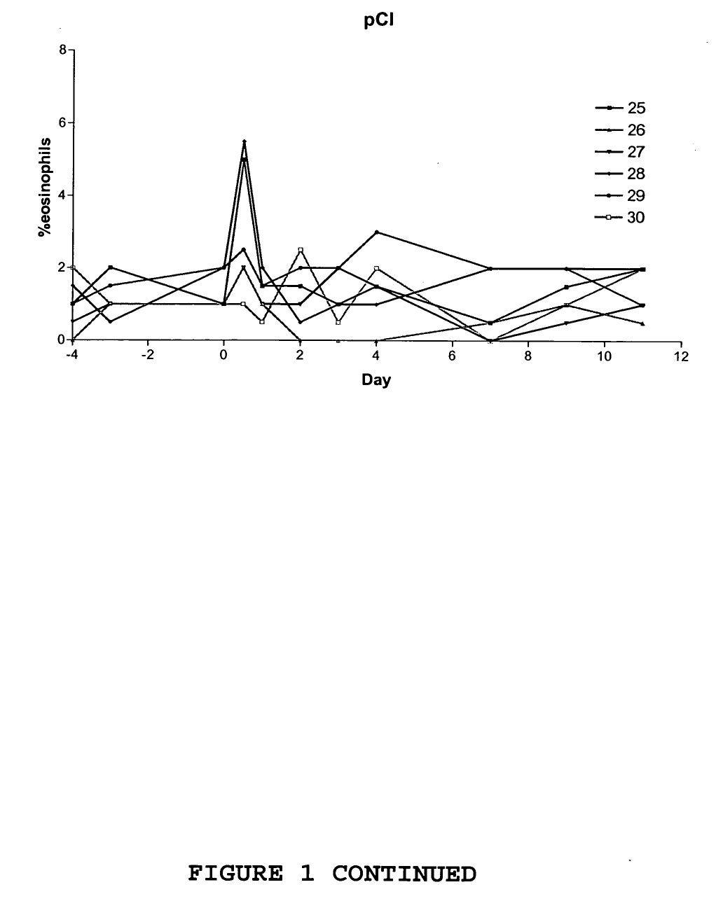

FIG. 1 shows the percentage of eosinophils in white blood cell (WBC) counts from blood taken from pigs for each treatment group. It can be readily seen that recombinant IL-5 resulted in a sustained increase in eosinophil numbers in blood over several days, with two doses being more effective than one dose. There was a variation in eosinophil responses between pigs with each treatment group ie. High and low responders and a biphasic response was also evident. One other conclusion drawn was that recombinant IL-5 was more effective than DNA in increasing eosinophil numbers.

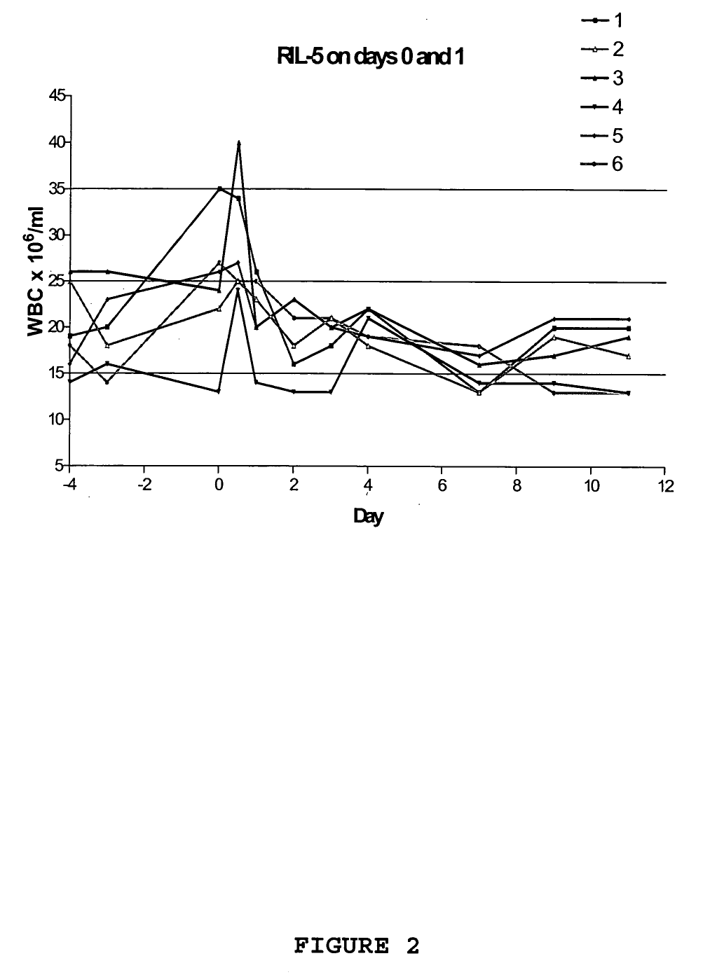

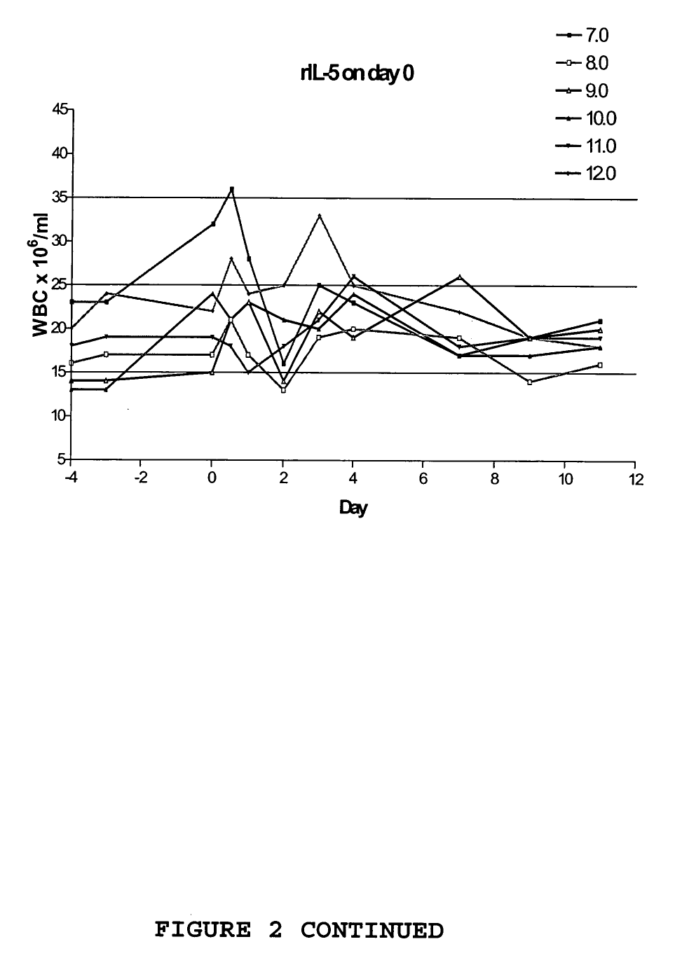

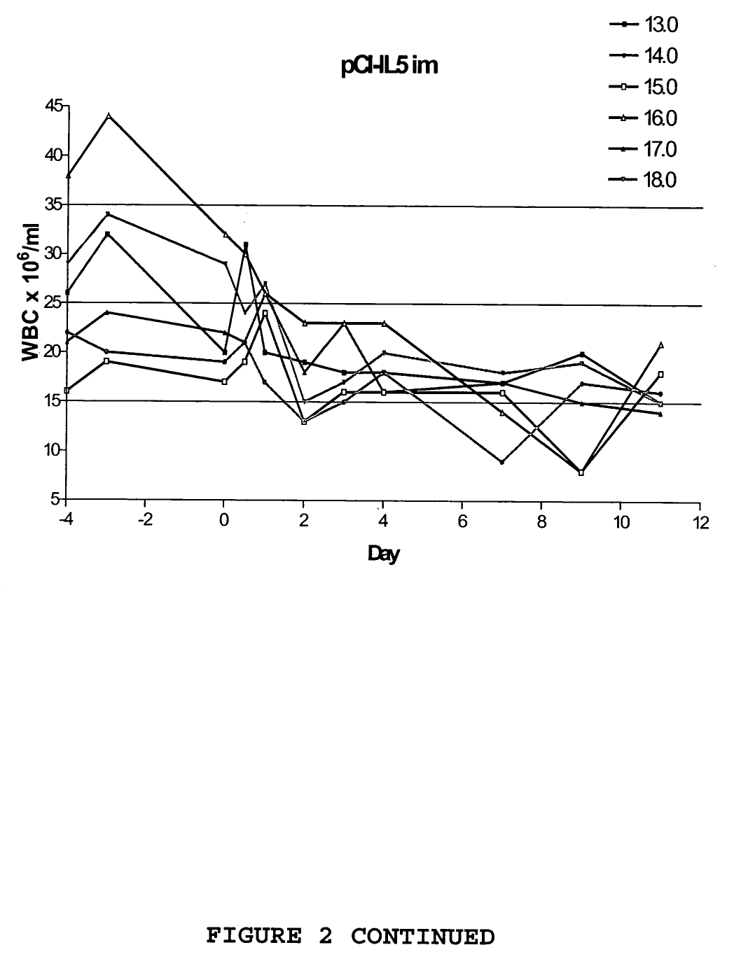

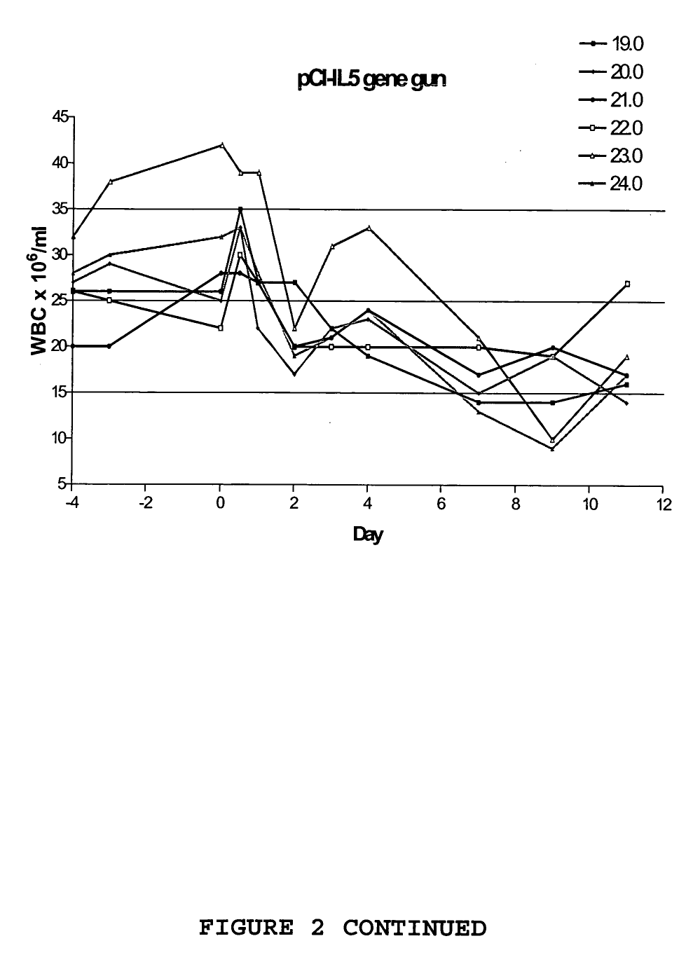

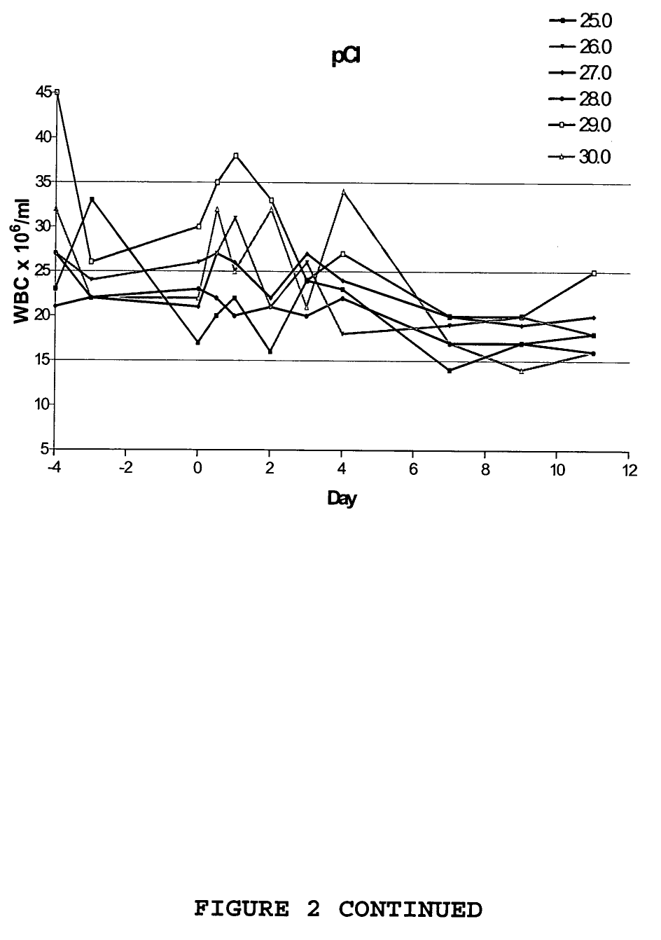

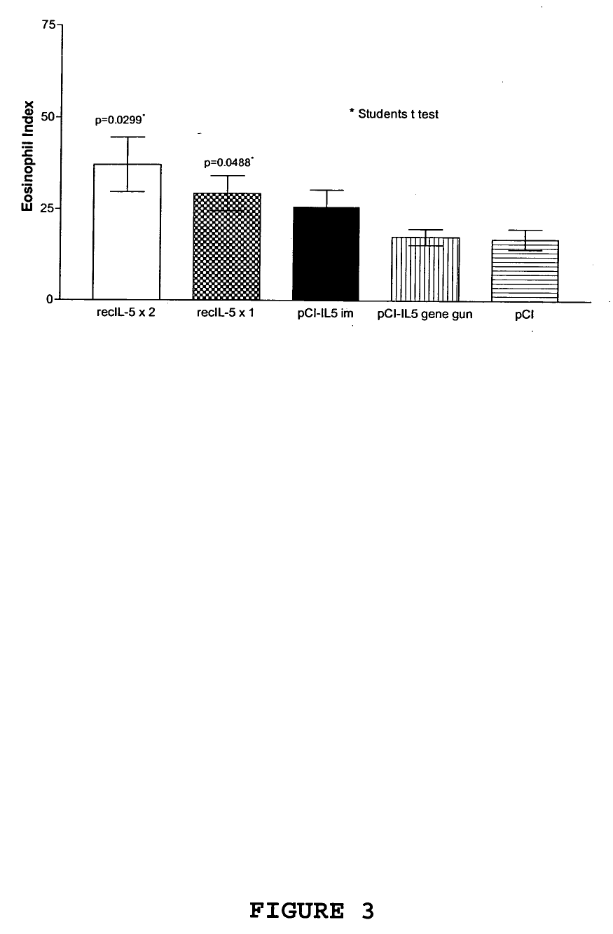

FIG. 2 shows that there was no significant difference between groups in terms of WBC counts, while FIG. 3 shows eosinophil index (statistical analysis) of increases in percentage eosinophils of WBC compared to the control and reveals that the recombinant IL-5 is more effective than DNA in increasing eosinophils, and two doses of recombinant protein more effective than 1. The analysis used a Prism statistical package that measured the area under the curve. Furthermore, FIG. 3 shows that genegun delivery of IL-5 was similar to the pCI parent plasmid control in terms of eosinophil responses.

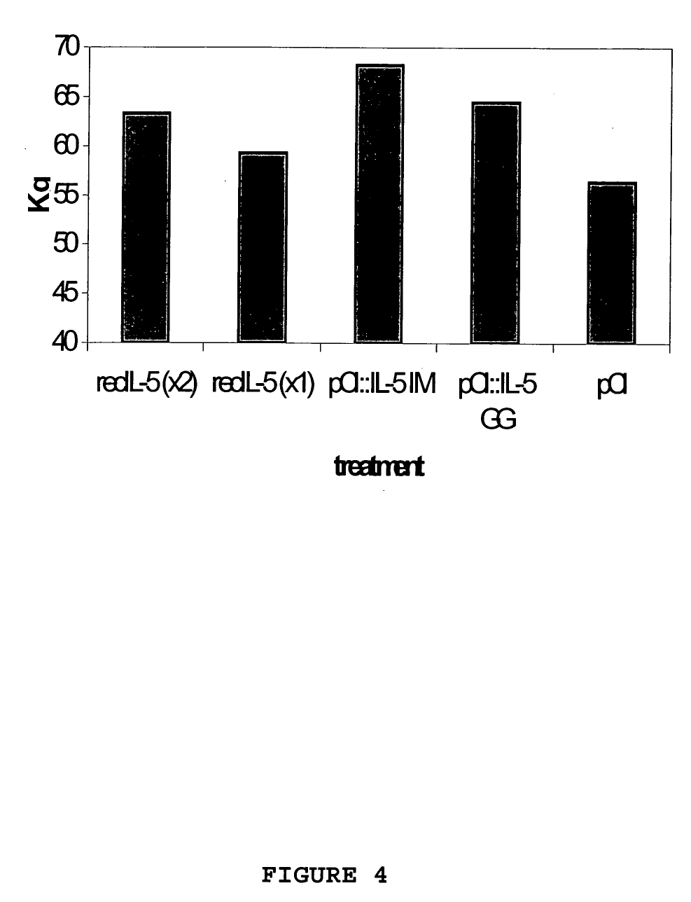

As shown in FIG. 4 there were increases in total group weight gain (over 16 days after initial treatment) of all IL-5 treated groups compared to the pCI DNA control group (5 to 20% increases). Interestingly, DNA delivered IL-5 appeared to have a greater effect on weight gain than recombinant IL-5. This result suggests that continuous administration of IL-5 mediated by expression of IL-5 DNA could be more effective.

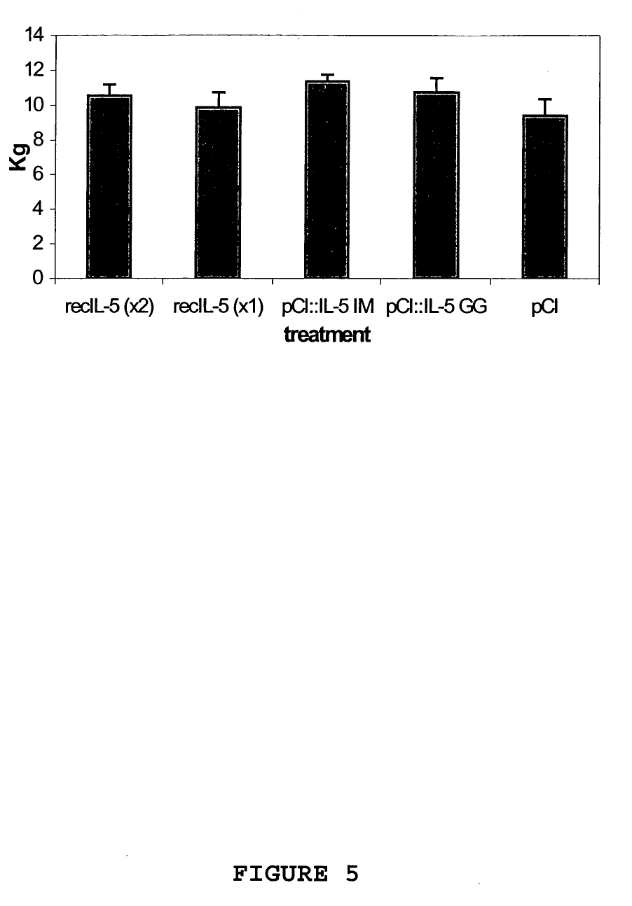

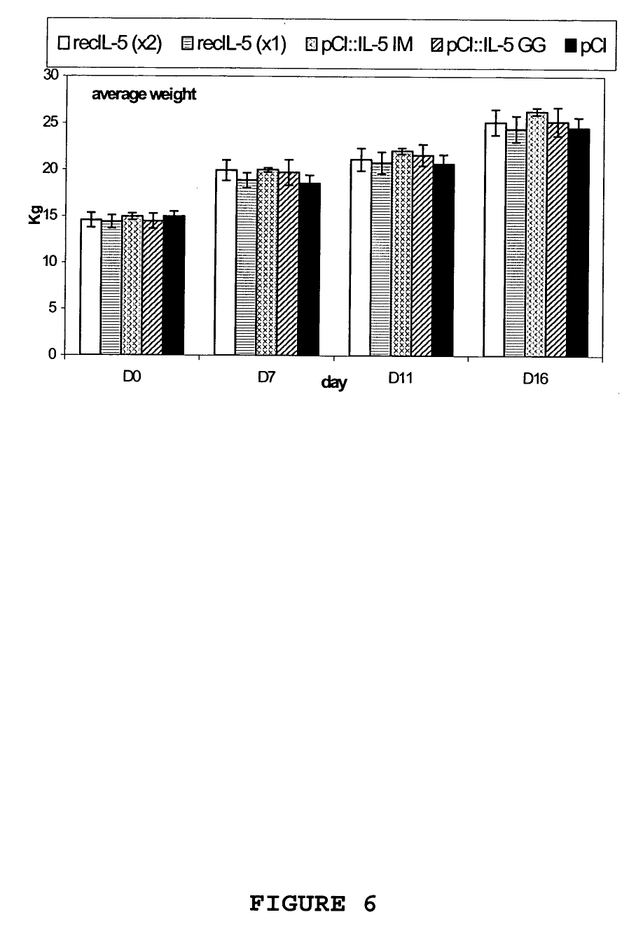

FIGS. 5 and 6 show that pigs treated with IL-5 had higher average weights than the controls (pCI). This is shown as the final average weight (FIG. 5) or throughout the trial (FIG. 6). Increases in weight gain (over 16 days after initial treatment, (FIG. 6) was more evident with IL-5 DNA treated pigs, and all IL-5 treated pigs have higher final average weight gain compared to the pCI DNA control group.

The trial clearly showed that the administration of recombinant IL-5 resulted in a sustained increase in eosinophil numbers (percentage of WBC) and that two doses was better than one. Although the DNA administration of IL-5 did not produce the same level of response as the recombinant protein, eosinophil number increased with IM delivery. The mode of delivery maybe important as genegun delivery delivers DNA to the skin surface and immediately underneath whereas IM is clearly into tissue (muscle).

Although, the experiment was conducted using medicated feed in an experimental environment, there were slight to moderate improvements in the weight gain of pigs (5 to 20% increases in weight gain compared to the pCI control). This result indicated that IL-5 could act as a growth promoting agent (as there was no overt disease or infection evident but no measurement of microbial load was made).

FIGS. 4 to 6 all show a general increase in weight gain (over 16 days) and total weights for those groups administered with IL-5, with a trend that DNA delivered IL-5 resulted in increased weight gain compared to recombinant IL-5, which in turn is higher than the DNA control.

The effect of IL-5 on blood eosinophil numbers was transient (several days).

### EXAMPLE 2

### Effect of High Dose and Multiple Dose IL-5 on Eosinophil Numbers in Blood

This trial compared 2 doses of recombinant IL-5 (100 μg/500 μg) and compared multiple injections (x1/x2/x4) of one dose (100 μg) to elevate eosinophil numbers.

Experimental Design

| Treatments | Administered |
|---|---|
| 1. 1 ml Saline | IM hind leg D0 |
| 2. 100 μg rec.IL-5 | IM hind leg D0 |
| 3. 500 μg rec.IL-5 | IM hind leg D0 |
| 4. 100 μg rec.IL-5 | IM hind leg D0, D1 |
| 5. 100 μg rec.IL-5 | IM hind leg D0, D1, D4, D7 |

Note:
D is the day of experiment
- means 6 pigs/treatment group. Blood haematology and weights measured.

The experiment was conducted using medicated feed Barastoc EziWean 150 then Bunge Grolean CREEP ad libitum in an experimental environment (PC2 containment facilities). Blood total and differential counts were performed using a CellDyn 3700 haematology machine.

Protocol Undertaken

| | |
|---|---|
| Day-4 | Bleed for haematology |
| Day-3 | Bleed for haematology, and weigh |
| Day 0 | Prebleed for haematology and dose with all |
| | treatments (1, 2, 3, 4, 5 as shown above) |

8 hrs later, bleed for haematology

| | |
|---|---|
| Day 1 | Bleed for haematology, Re-dose treatments 4 and 5. |
| Day 2 | Bleed for haematology |
| Day 3 | Bleed for haematology |
| Day 4 | Bleed for haematology, Re-dose treatment 5. |
| Day 7 | Bleed for haematology, Re-dose treatment 5. |
| Day 9 | Bleed for haematology |
| Day 11 | Bleed for haematology |
| Day 14 | Weigh. |

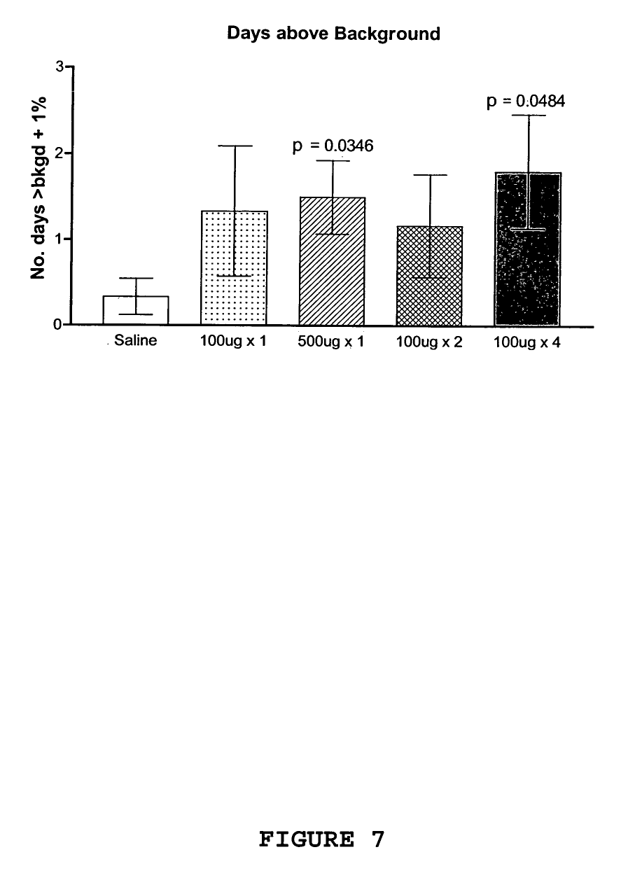

From FIG. 7 it can be seen that IL-5 administration increased eosinophil numbers in blood in terms of percentage of WBC. The analysis used a Prism statistical package that measured the area under the curve. The higher dose and multiple doses were statistically significant, with 4 injections of 100 μg (Days 0, 1, 4, 7) being more effective than a single injection of a higher single dose (500 μg). 1 and 2 doses of 100 g IL-5 were comparable in terms of elevating the percentage circulating eosinophils. EXAMPLE 3

### Effect of Mode of Administration

This trial compared the delivery of IL-5 as recombinant protein or by DNA using different routes of administration. Each treatment was administered six times over 2 weeks (Days 0, 1, 3, 6, 8, 10).

Experimental Design

| Treatments | Administered |
|---|---|
| 1. 100 μg rec.IL-5 | IM hind leg |
| 2. 100 μg rec.IL-5 | intranasal spray using puffer |
| 3. 200 μg pCI::IL-5 | DNA IM hind leg |
| 4. 6 μg pCI::IL-5 | IM genegun, on belly and hind legs |
| 5. Saline | IM hind leg |

6 pigs/treatment group. Blood haematology measured.

The experiment was conducted using medicated feed Barastoc EziWean 150 then Bunge Grolean CREEP ad libitum in an experimental environment (PC2 containment facilities).

Blood total and differential counts were performed using a CellDyn 3700 haematology machine.

Protocol Undertaken

| | |
|---|---|
| Day 0 | Weigh, Prebleed for haematology and dose with all |
| | treatments (1, 2, 3, 4, 5) |
| Day 1 | Bleed for haematology, Re-dose all treatments. |
| Day 2 | Bleed for haematology |
| Day 3 | Bleed for haematology, Re-dose all treatments. |
| Day 6 | Bleed for haematology, Re-dose all treatments. |
| Day 7 | Bleed for haematology |
| Day 8 | Bleed for haematology, Re-dose all treatments. |
| Day 9 | Bleed for haematology |
| Day 10 | Bleed for haematology, Re-dose all treatments. |
| Day 13 | Bleed for haematology and serum. |
| Day 15 | Bleed for serum. |
| Day 21 | Bleed for haematology and serum. |

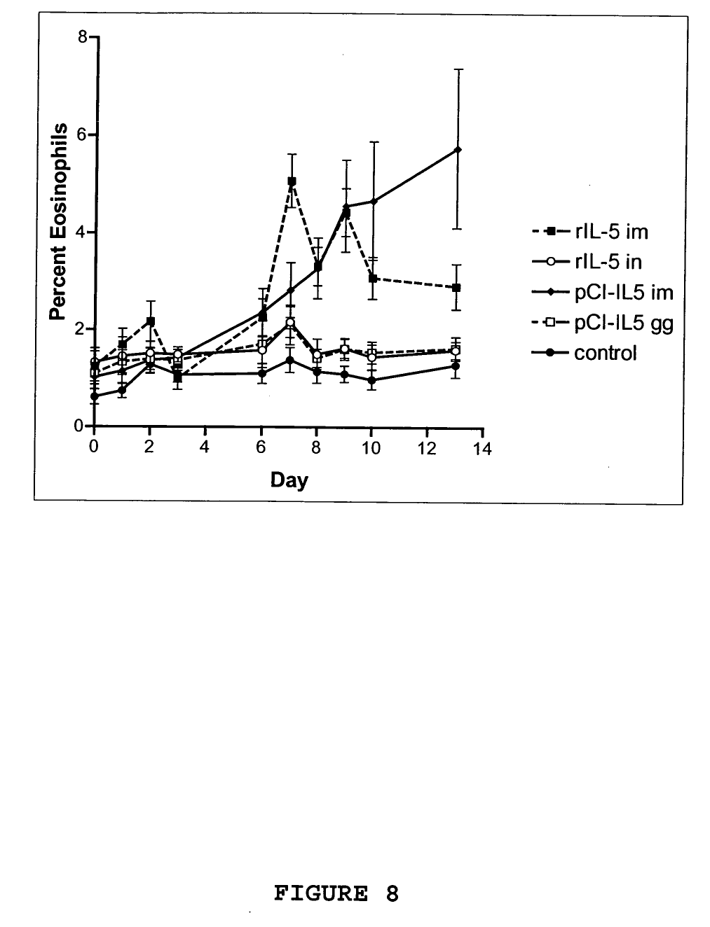

As can be seen from FIG. 8 the effects of multiple administration of recombinant IL-5 IM and DNA IL-5 IM was an increase in the duration and level of eosinophils in blood (percentage of WBC).

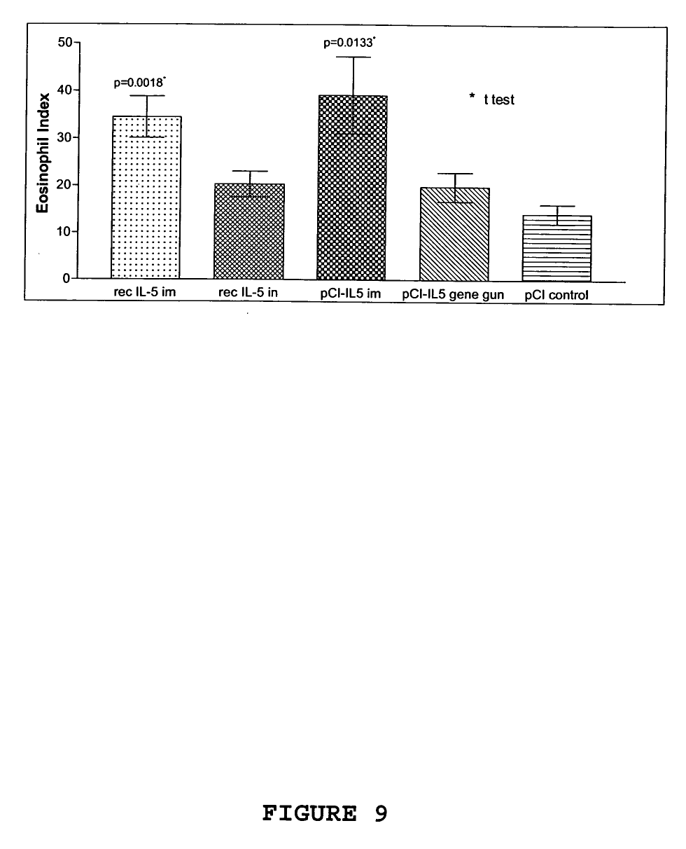

Recombinant IL-5 and IL-5 DNA injected intramuscularly six times over 2 weeks significantly (P<0.05 and P<0.01 respectively) elevated circulating eosinophil numbers. Nasal delivery of recombinant IL-5 and genegun delivered IL-5 DNA had slight increases on eosinophil numbers compared to the DNA control but not statistically significant (See FIG. 9). EXAMPLE 4

### Improved Growth Performance and Immunity of Pigs Administered IL-5

This trial evaluated the capacity of IL-5 to improve growth performance and immunity of pigs by comparing the growth rate and health of weaner pigs (28 day old weaners is Day 0 of the trial and the weaner period continued for 42 days) through to the finisher stage (Days 93 to 113) and slaughter (133 days after commencing the trial). The pigs were administered recombinant porcine cytokine, IL-5, and saline was used as a control, with and without standard weaner medicated water and feed in a commercial piggery environment.

Experimental Design

| Treatments | Administered |
|---|---|
| Saline | injection, needle IM neck muscle, |
| | twice weekly. |
| 100 μg IL-5 | (in saline) injection, needle IM |
| | neck muscle, twice weekly. |

40 pigs per treatment mixed in-groups of 20, with 4 replicates containing standard medicated water and feed and 4 replicates without medicated feed or water.

Overall weights for each group at the start of the experiment were equivalent. All pigs at the start of the trial (Day 0) were 28 day old male weaners.

IL-5 was provided in saline to inject one ml/pig.

Weights were measured at the start, weekly and at end of the experiment. The original proposal was designed for measuring weight over the weaner period; however, due to significant growth responses with IL-5 administration the measurement of weight continued to slaughter (pigs were 161 days old).

Blood and sera samples collected at the start (before treatments) and end of the weaner period. Blood and sera were taken prior to injecting samples.

Recombinant porcine IL-5 was expressed in E. coli and purified using a polyHis tag system as described in Qiagen Inc, USA, instructions and Clontech, USA, Talon Manual instructions. IL-5 was tested for biological activity in a bioassay prior to the start of the experiment.

Protocol Undertaken

| | |
|---|---|
| Day 0 | Weighed and grouped 28 day old |
| | weaners. |
| Day 1 | Bleed. Injected Groups A, B. |
| Day 6 | Injected Groups A, B. |
| Day 7 (Week 1) | Weighed |
| Day 9 | Injected Groups A, B. |
| Day 13 | Injected Groups A, B. |
| Day 14 (Week 2) | Weighed |
| Day 16 | Injected Groups A, B. |
| Day 20 | Injected Groups A, B. |
| Day 21 (Week 3) | Weighed |
| Day 23 | Injected Groups A, B. |
| Day 27 | Injected Groups A, B. |
| Day 28 (Week 4) | Weighed |
| Day 30 | Injected Groups A, B. |
| Day 34 | Injected Groups A, B. |
| Day 35 (Week 5) | Weighed |
| Day 37 | Injected Groups A, B. |
| Day 41 | Injected Groups A, B. |
| Day 42 (Week 6) | Weighed. Final bleed. Moved to |
| | grower pens. |
| (Day 42) | Pigs from highest and lowest growth |
| | groups tested for vocalisation score |
| | (Giles and Furley 1999: Proc. 26^{th} |
| (Days 42-93) | Annual Conference of the |
| | Australasian Society for the Study |
| | of Animal Behaviour. University of |
| | New England, Armidale Australia. |
| | P. 17) to determine correlation. |
| (Days 93-133) | Grower stage. ALL pigs given |
| | standard feed and remained in |
| | previous groups. Weighed during |
| | (D73) and end of grower stage (D93). |
| | Finisher stage. Pigs moved into |
| | single pens and feed intake measured |
| | for FCR (food conversion ratio). |
| | All pigs given standard finisher |
| | feed. Weighed during (D114) and end |
| | of finisher stage (Slaughter D133). |
| | Measured backfat, carcass weight, |
| | % dressing. |

Notes:
+ medicated (plus antibiotics) feed and water.
− unmedicated (no antibiotics) feed and water.
A & B = cytokine treatment and saline treatment.

At the start of the trial, the mean weights and variance for each group were equalised. Thus, the increase in total or average weights witnessed in the IL-5 medicated group was due to IL-5 administration and not simply due to differences between weights at the start of the trial.

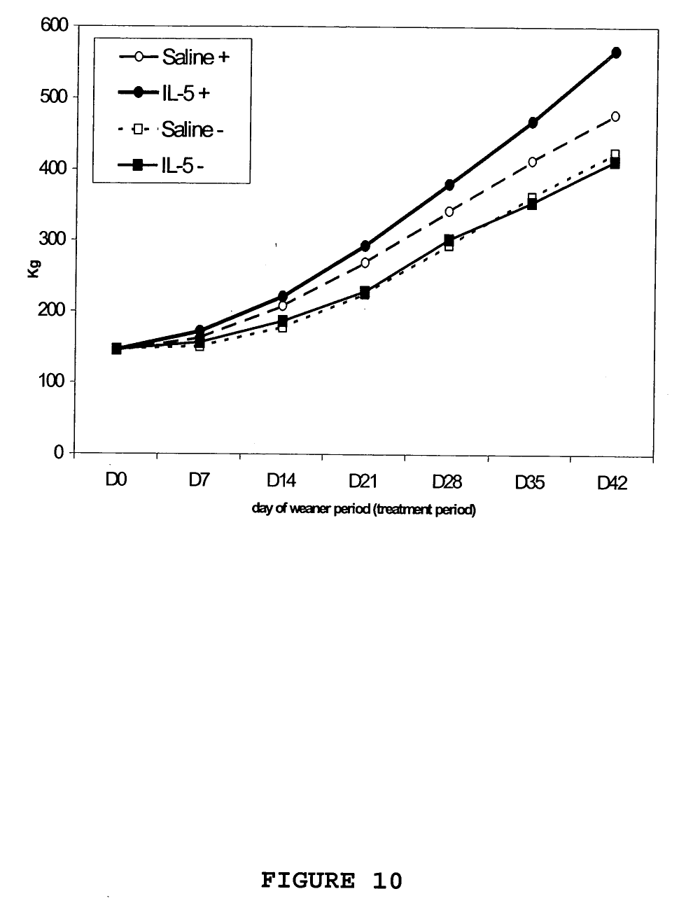

FIG. 10 shows that the total weights of each group over the weaner period represented a combination of weight gained and the number of pigs remaining in each group. These are possibly a reflection of resistance to infections as all pigs were subjected to a severe exposure of pathogens (deaths occurred from H. parasuis “Glasser's”, APP and Swine dysentry).

Antibiotic medicated pigs have consistently higher combined weights than un-medicated pigs (saline un-medicated group weight is 89% of saline medicated group weight).

IL-5 medicated group has a consistently and significantly higher total combined weight than the medicated control groups (Week (W) 1 5.5%, W2 6.3%, W3 8.8%, W4 11.1%, W5 13.4%, W6 18.6% higher than saline medicated group, representing 89 kg over 6 weeks for a group of 20 pigs i.e. During treatment period).

Graphs of group weights are indicative that weight differences between IL-5 group and medicated saline controls increased further during the grower and finisher stages (see results below).

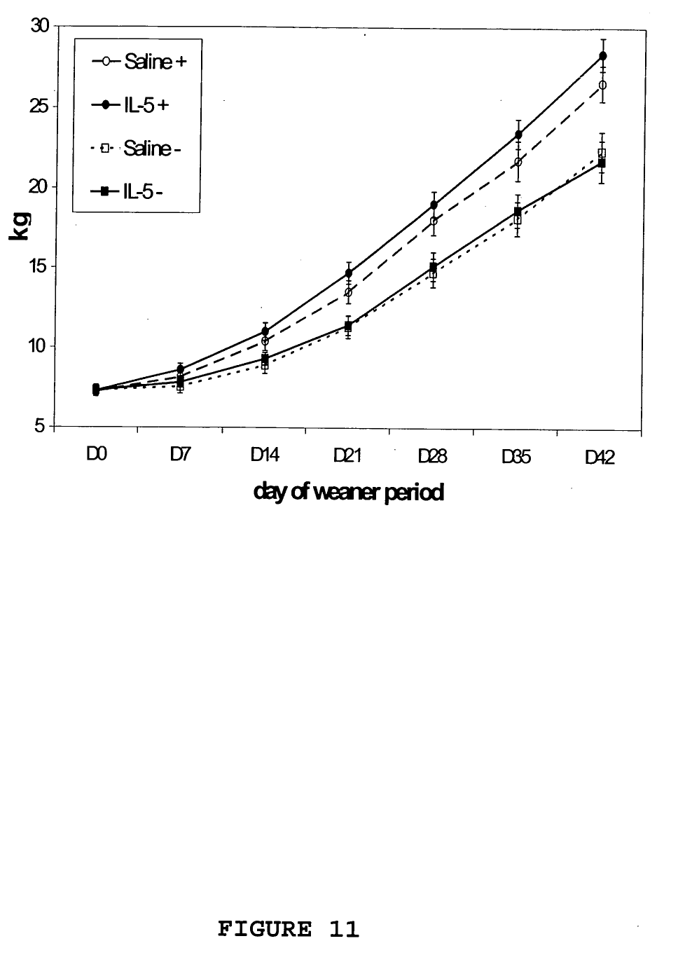

FIG. 11 shows the average weight of individual pigs in each group during weaner period. It should be noted that deaths usually occurred with pigs of lower weights, which resulted in slightly higher average weights in these groups.

The IL-5 medicated group had a consistently higher average weight over the 6-week period compared to saline medicated controls and appears to be increasing with time (W1 5.5%, W2 6.3%, W3 8.8%, W4 5.5%, W5 7.8%, W6 6.8%).

The IL-5 medicated group had a consistent increase over the saline medicated control in terms of weight gain per pig and rate of gain (ROG) during the administration of IL-5 (ROG: W1 45.6%, W2 19.6%, W3 18.2%, W4 8.8%, W5 11.3%, W6 9.1%).

The IL-5 un-medicated group has a consistently small increase in average weights over the un-medicated saline controls except for the last week (Week 6).

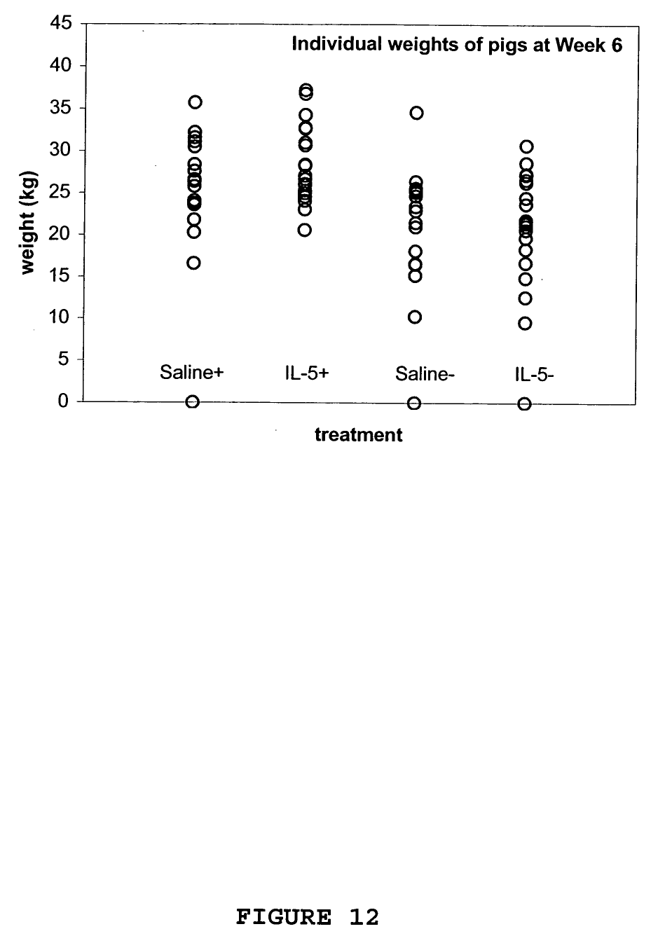

IL-5 has a significant effect when combined with medicated feed and water presumably acted as a growth promoter. IL-5 may also act as an immune stimulant as there were no deaths in the group with antibiotics (described later). The growth performance of pigs in the IL-5 medicated group was more consistent than all other groups (narrower range and higher weights in general, FIG. 12). This was another beneficial effect of IL-5 administration.

It appeared that the weights of smaller pigs in particular have been increased with IL-5 administration.

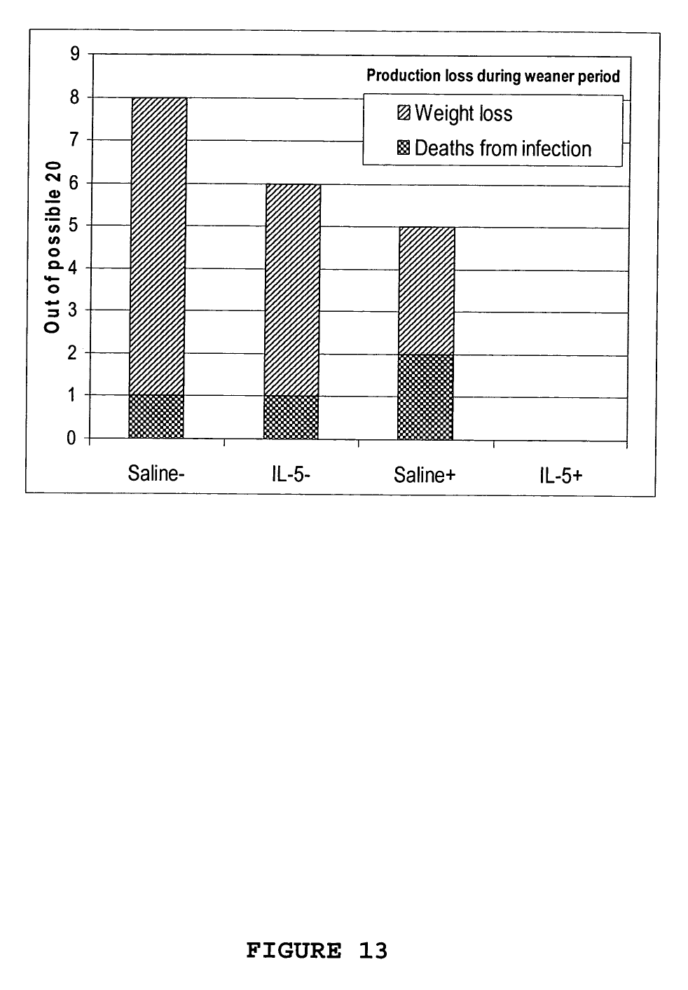

The production loss in terms of deaths from infectious disease or weight loss as measured by weekly weighing is shown in FIG. 13. No deaths occurred in the IL-5 medicated group (FIG. 13), a factor that influenced the positive effect of IL-5 on increasing total group weight as described above (FIG. 10). The majority of deaths also included prior weight loss, but were recorded as deaths only. The antibiotic medicated groups had reduced numbers of pigs that lost weight during 1 or more weeks during the weaner period compared to the un-medicated groups. The first week after weaning was where the majority (>80%) of pigs lost weight. A significant conclusion from these results was that no pig from the medicated IL-5 treatment group lost weight or died from infectious disease. IL-5 treatment in the un-medicated groups also reduced production loss.

Table 2 shows the autopsy report for the above trial.

TABLE 2
AUTOPSY REPORT SUMMARY

| Groups | Deaths |
|---|---|
| Saline, unmedicated | 1x H. parasuis (Glasser's |
| | disease) |
| Saline, medicated | 1x H. parasuis, 1x Swine |
| | dysentry, 1x bleeding trauma |
| IL-5, unmedicated | 2x H. parasuis |
| IL-5, medicated | No deaths or intervention |
| | treatments required |

The conclusions from Table 2 are that there were only 6 deaths from 80 weaners (5 from infectious disease, 1 from bleeding trauma). Pigs in IL-5 medicated group reported to be in excellent condition and health compared to other groups.

Blood was taken the day after IL-5 and saline treatments and represent a single time point only. As shown in FIG. 14 IL-5 treatment had a substantial effect on % eosinophil cells of WBC in both the medicated and un-medicated groups. As a general observation IL-5 plus medication had higher eosinophils numbers than the IL-5 without antibiotics.

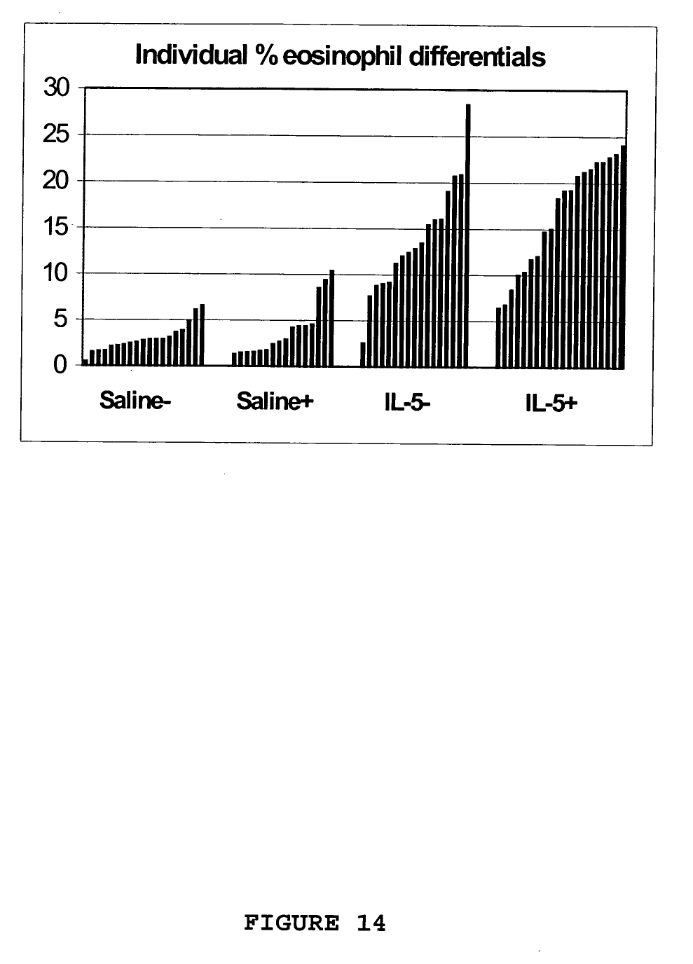

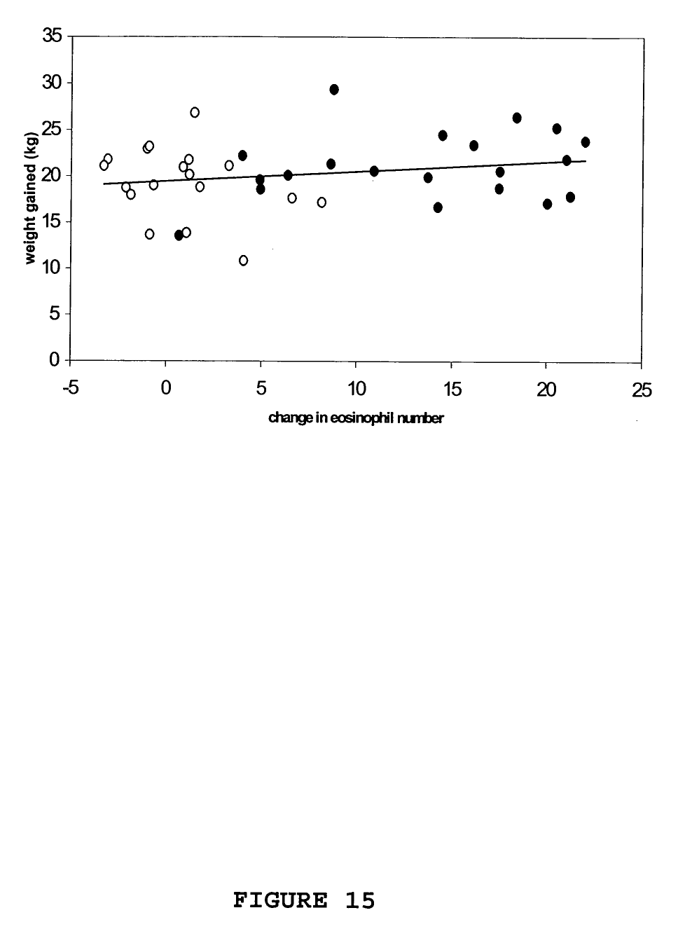

Although IL-5 increased the % eosinophils of blood in both the medicated and un-medicated treatments, an improvement in growth (measured as rate of growth or average daily gain or average weight gain or total weight) over saline controls was higher for the IL-5 plus antibiotics than for the IL-5 without antibiotic. This indicated that an increase in % eosinophils alone may not be the mechanism of growth promotion. FIG. 15 shows that there is a positive relationship between absolute eosinophil numbers and growth rate.

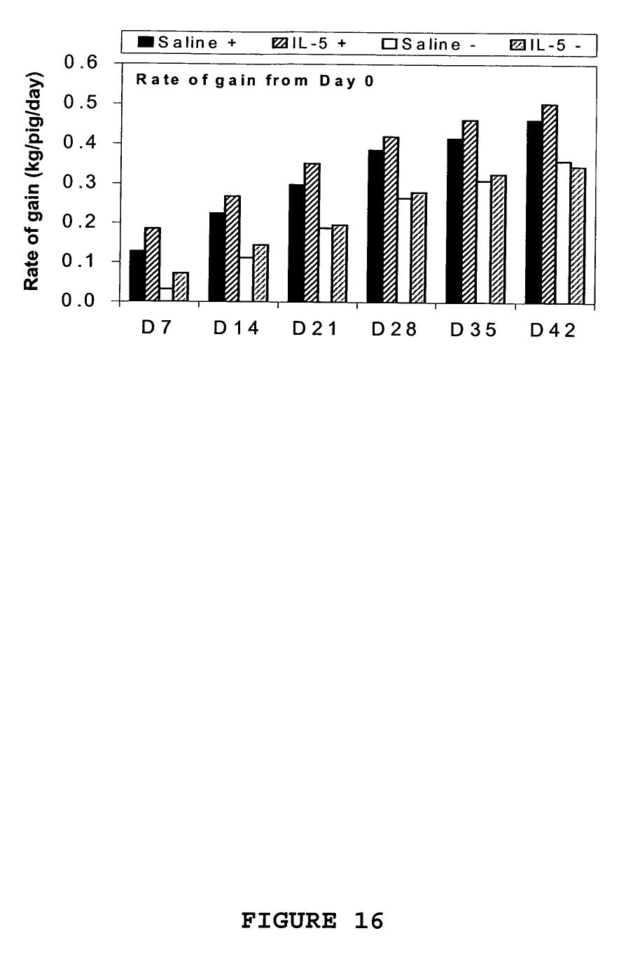

Rate of gain over the weaner period is shown in FIG. 16. It was observed that similar to average weight, total weight and weight gain, IL-5 consistently increased the rate of gain in the medicated pigs.

The rate of gain was consistently higher in the medicated groups compared to the un-medicated groups, with the rate of gain for the un-medicated IL-5 group generally higher than the un-medicated saline control.

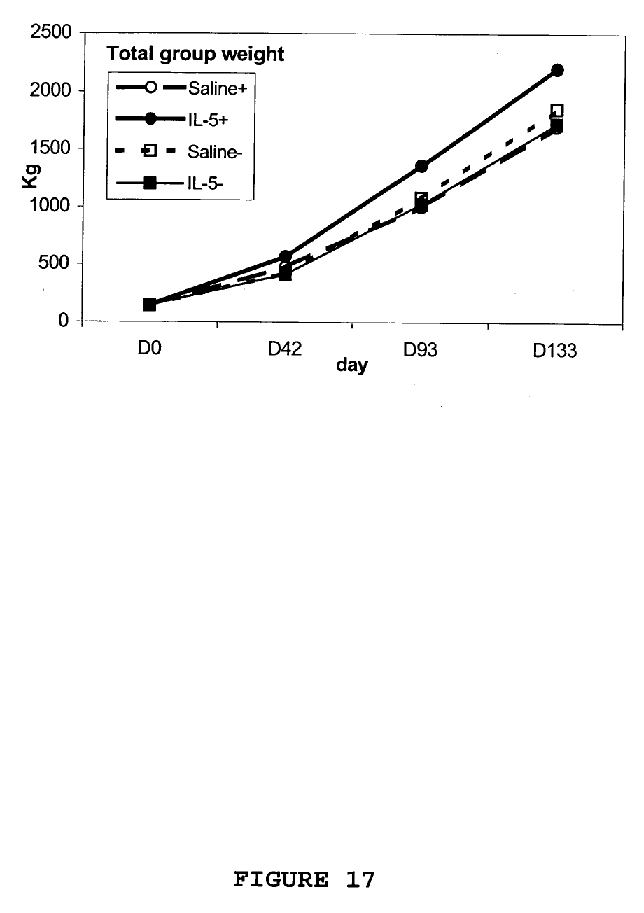

The total weights of all pigs in the treatment groups over the entire production period are shown in FIG. 17. It is evident that the IL-5 medicated group has a significant increase in total weights compared to all other groups. This increase appears to be a combination of a higher average weight gain (or Rate of Gain or Average Daily Gain) and no deaths for the IL-5 medicated group. The increase in total weights of the IL-5 medicated group at the end of the weaner period continued to slaughter.

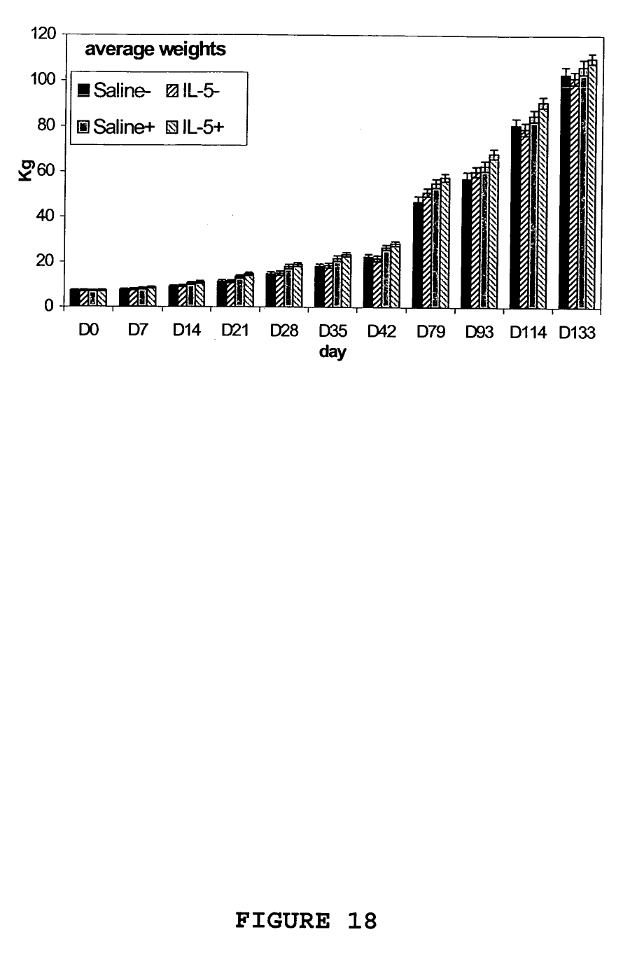

The average weights of individual pigs in treatment groups during trial are shown in FIG. 18. IL-5 medicated group had consistently higher average weights than all other groups, while the medicated pigs generally have higher average weights than un-medicated groups. Deaths typically occurred in pigs of lower than average weight that would artificially increase the average weight of the groups.

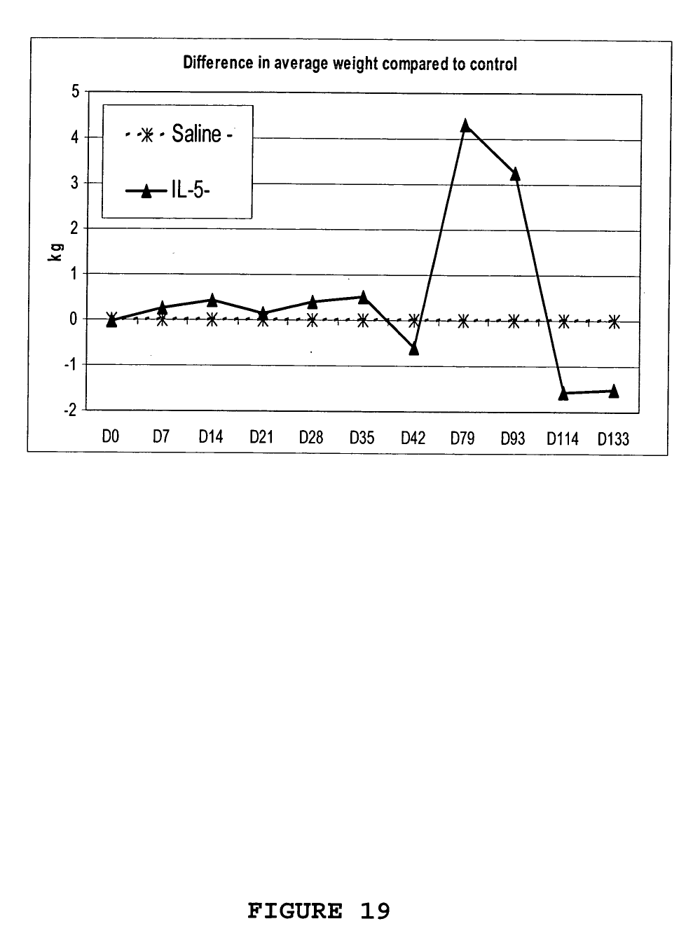

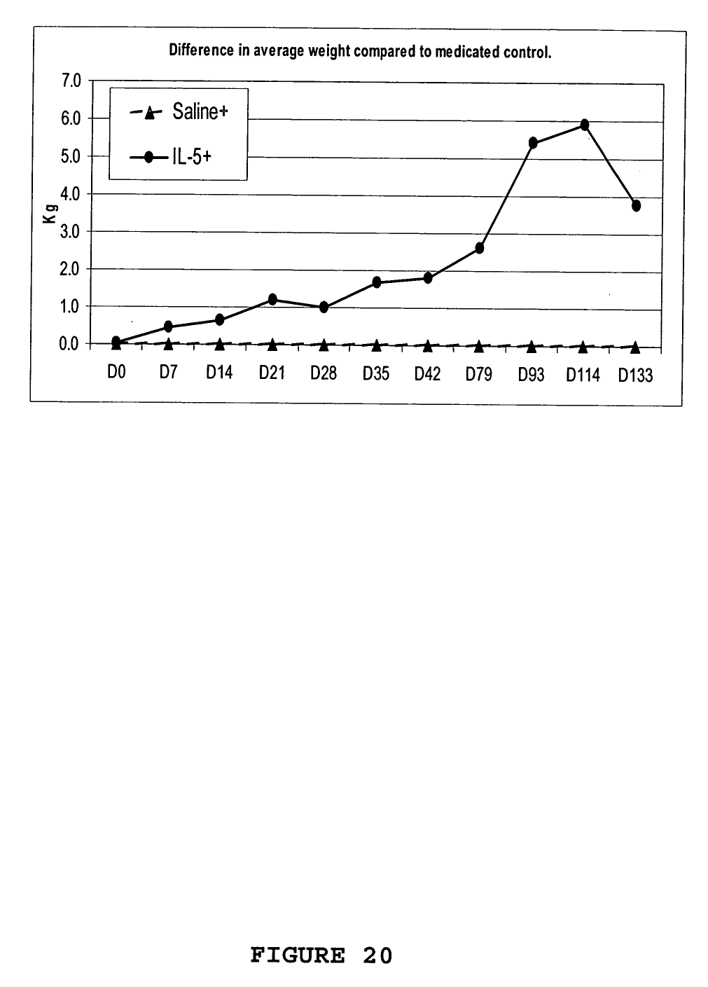

The IL-5 treated pigs have higher average weights than the respective saline controls with each antibiotic regime at nearly all time points (FIGS. 19 and 20). Regardless of antibiotic supplements, pigs treated with IL-5 had consistently higher average weight gains during the Weaner and Grower periods compared to the respective saline controls. This trend did not continue during the finisher period where the difference between average weights diminished.

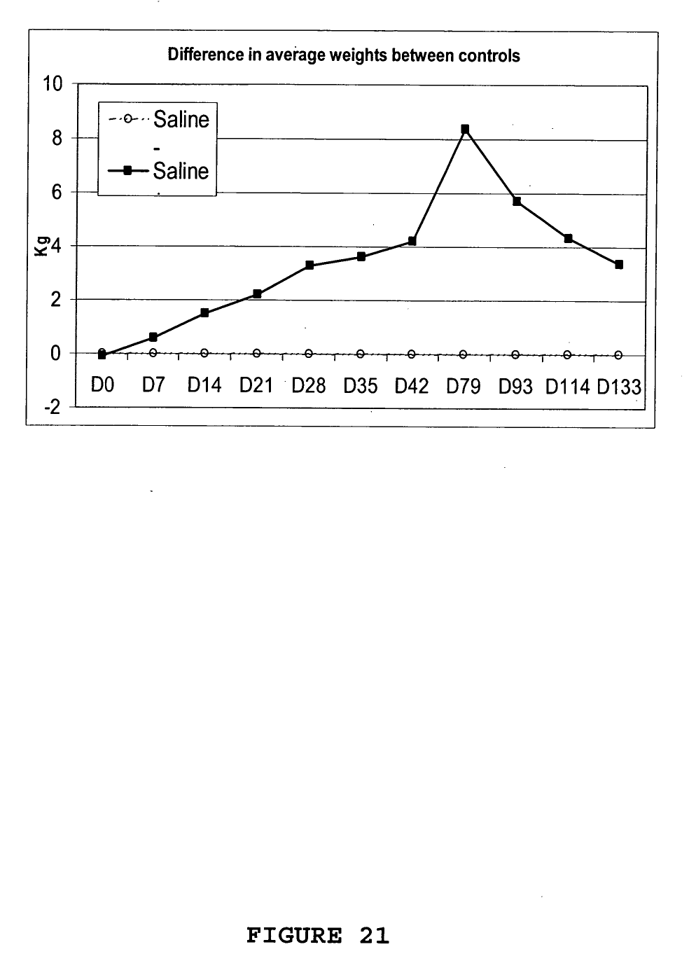

The results also showed that antibiotic supplements result in consistently higher average weight gains during the Weaner, Grower, and Finisher periods compared to the saline control without antibiotic supplements in water (FIG. 21). Again, these differences in average weights also diminished during the Finisher period. The reason for this remains unknown; however, there are two compounding events that may have had an impact:

1). Antibiotics were withdrawn during the weaner period only, and provided during the Grower and Finisher periods. Hence, antibiotics may have increased the growth rate or health of the pigs in the non-medicated group.

2). Pigs were transferred to individual pens at the start of the Finisher period (D93). Although this is not standard practice for commercial piggeries, but undertaken to obtain food conversion ratio data.

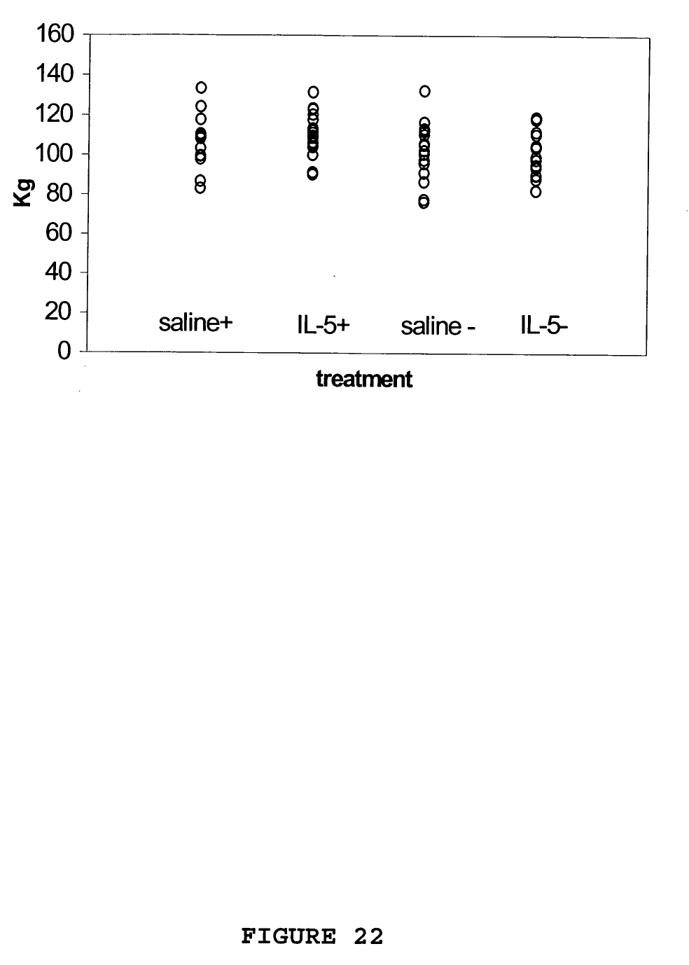

Treatment with IL-5 reduced variation in individual pig weights up to slaughter (FIG. 22), probably by lifting the weight of smaller pigs.

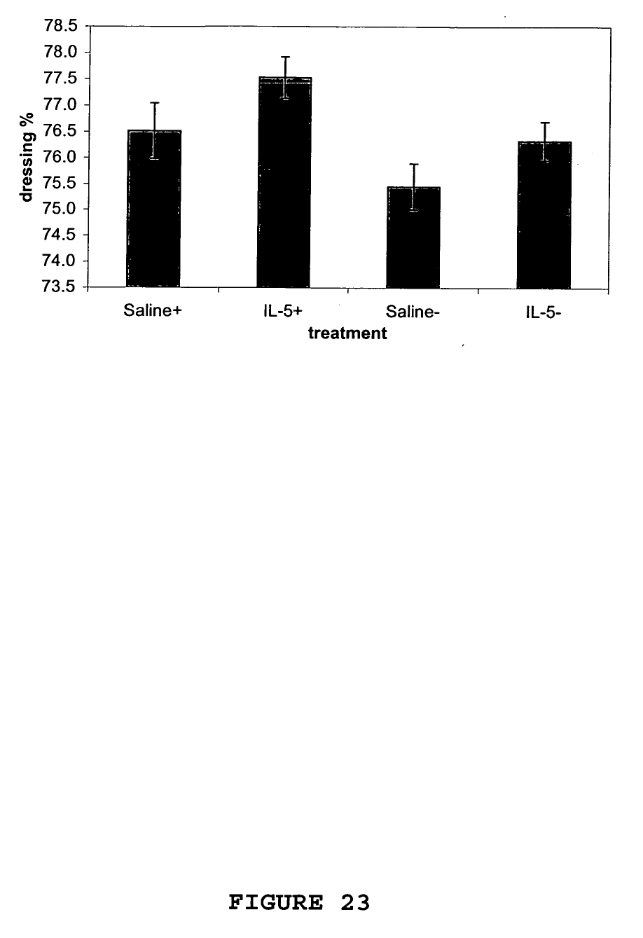

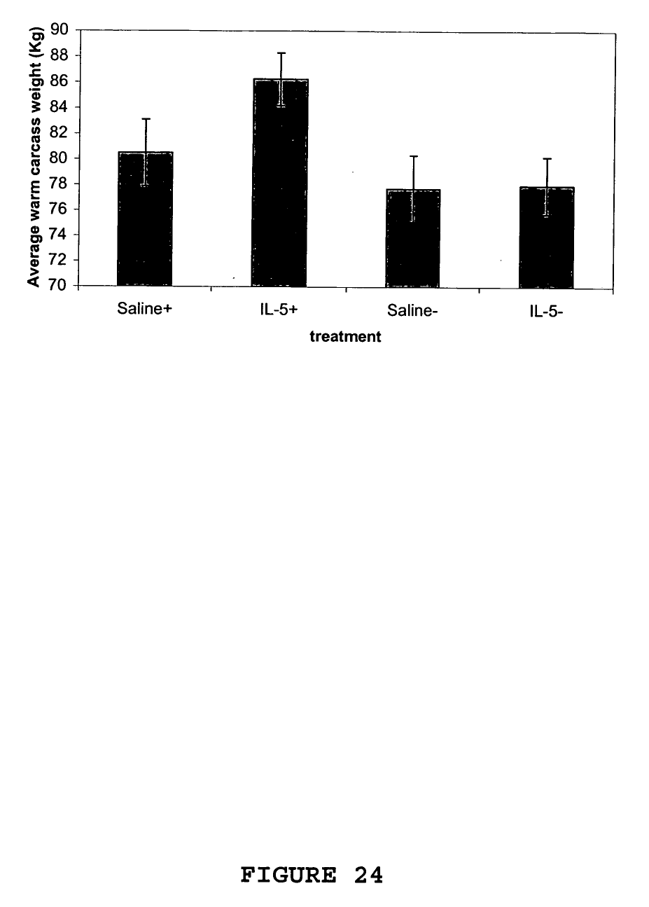

IL-5 treatment was found to have a statistically significant effect (p<0.045) on the dressing percentage of carcasses at slaughter (FIG. 23). IL-5 treatment improved dressing percentage regardless of antibiotic administration. Results for warm carcass weight are shown in FIG. 24. IL-5 increased warm carcass weight compared to saline controls when pigs were medicated with antibiotics. However, this effect of IL-5 was not as obvious in pigs without antibiotics.

These results indicate that IL-5 treatment has a positive effect on slaughter characteristics by increasing dressing percentage under both medication regimens, and increasing warm carcass weight in the presence of antibiotic supplementation.

### EXAMPLE 5

### Repeat of the Growth Performance and/or Immunity of Pigs Administered IL-5

This trial repeats the evaluation of IL-5 to improve growth performance and/or immunity of pigs by comparing the growth rate and health of male and female weaner pigs (from 28 days old: Week 0 of the trial) through the weaner (Weeks 0-6), grower (Weeks 6-13) and finisher (Weeks 13-19) stages to slaughter (Week 19), which were administered with recombinant porcine cytokine IL-5, and saline was used as a control, with and without standard weaner medicated water and feed, and reduced antibiotics, in a commercial piggery environment.

This trial (weaner/grower/finisher trial) was designed to investigate the effect of providing IL-5 and the controls (saline) from weaning to slaughter with normal, reduced and no antibiotics in water supply. The experiment evaluates the capacity of IL-5 for replacing antibiotics under commercial pig rearing conditions and to determine the effect of continuous administration of the cytokines throughout the life of the pig on performance and carcass characteristics.

Experimental Procedures

The experiment was undertaken in a commercial environment where the pigs were weaned at 28 days of age. All injections were 1 ml. There were 16 pigs per treatment, 8 males and 8 females per treatment. The overall weight of pigs in each treatment was similar at the start of the experiment.

Treatment protocol

| Group | Treatment | Administration | Medicated | feed/water |
|---|---|---|---|---|
| 1. | Saline | Weaner/Grower/Finisher | | 0 |
| 2. | Saline | Weaner/Grower/Finisher | | Reduced |
| 3. | Saline | Weaner/Grower/Finisher | | Normal |
| 4. | IL-5 | Weaner/Grower/Finisher | | 0 |
| 5. | IL-5 | Weaner/Grower/Finisher | | Reduced |
| 6. | IL-5 | Weaner/Grower/Finisher | | Normal |
| 7. | IL-5 | Weaner (Control) | | Normal |

Group 7 is a repeat of the previous trial at the commercial piggery for comparison.

Symbols Used

−means no antibiotic supplements in water or feed throughout trial.

0.5 means single antibiotics used throughout trial at normal dose

+ means normal antibiotic regime used throughout trial IL-5+ means IL-5 administered during Weaner/Grower/Finisher periods.

IL-5+p means IL-5 administered during Weaner period only.

Treatments

A. Saline injection, 1 ml IM neck muscle.

B. 100 μg IL-5 injection, 1 ml IM neck muscle.

Weaner stage: 2 injections per week,

Grower and finisher stage 1 injection per week.

Pigs were weaned and weighted at the start of the experiment (D0, W0) and weekly until the end of the weaner period (W6), at the end of the grower (W13) and finisher (W19) stages and once during the grower (W9) and finisher (W16) stages. Blood and sera samples were collected at the start (before treatments) and end of the weaner, grower and finisher periods. Blood and sera were taken prior to treatment injection. Haematology (totals and differentials) was performed.

At the start of the trial, the mean weight and the variance of all groups was equalised to reduce confounding influence of starting weight. Thus, the positive effects on growth were due to treatment effects.

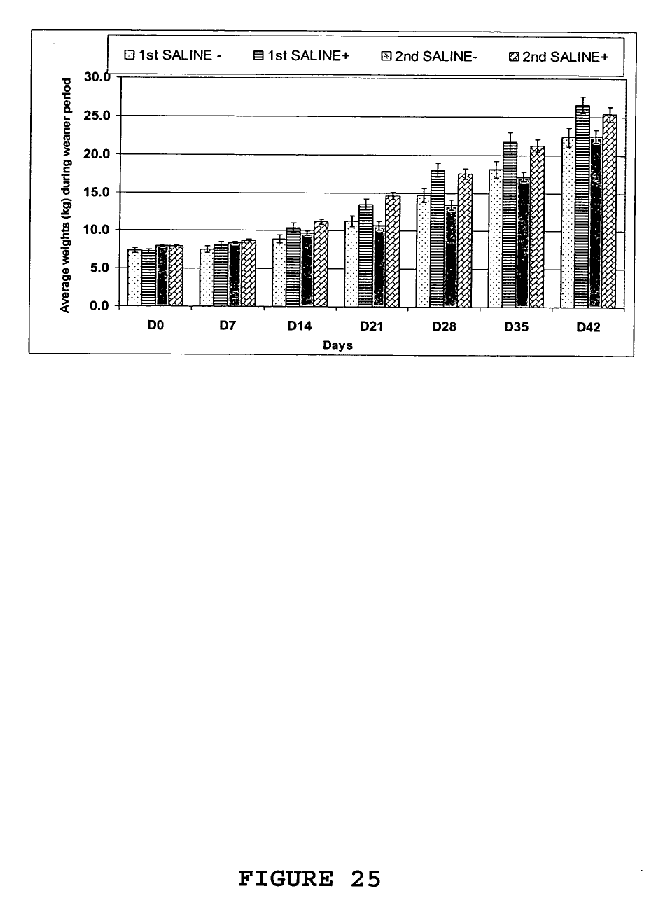

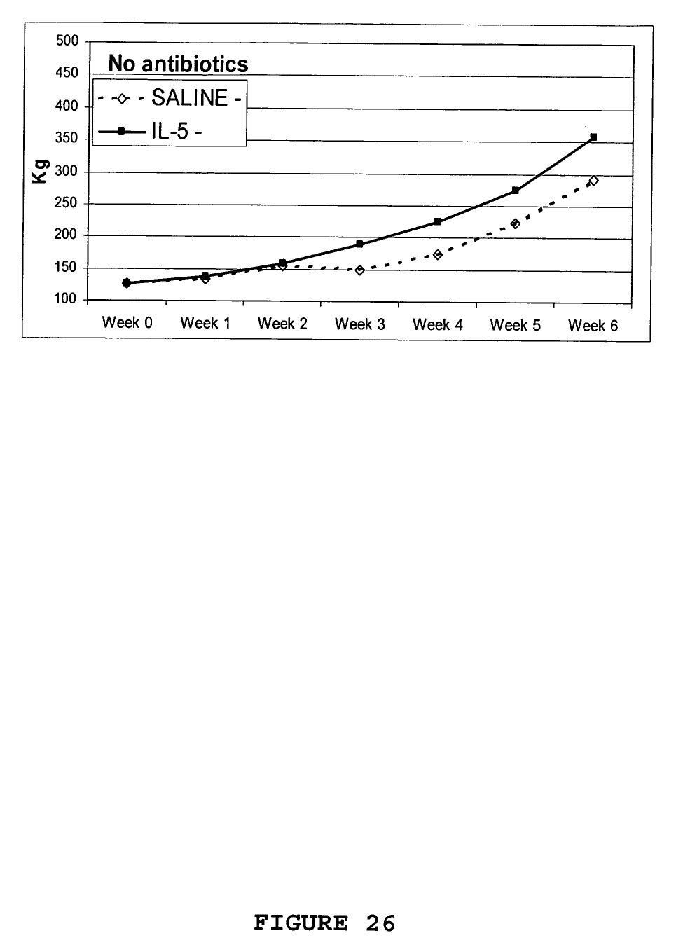

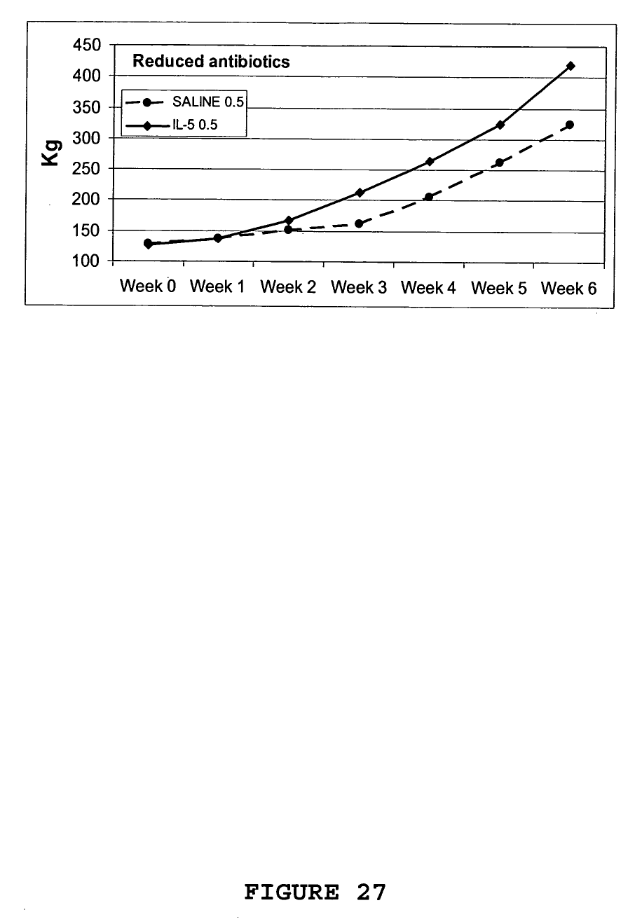

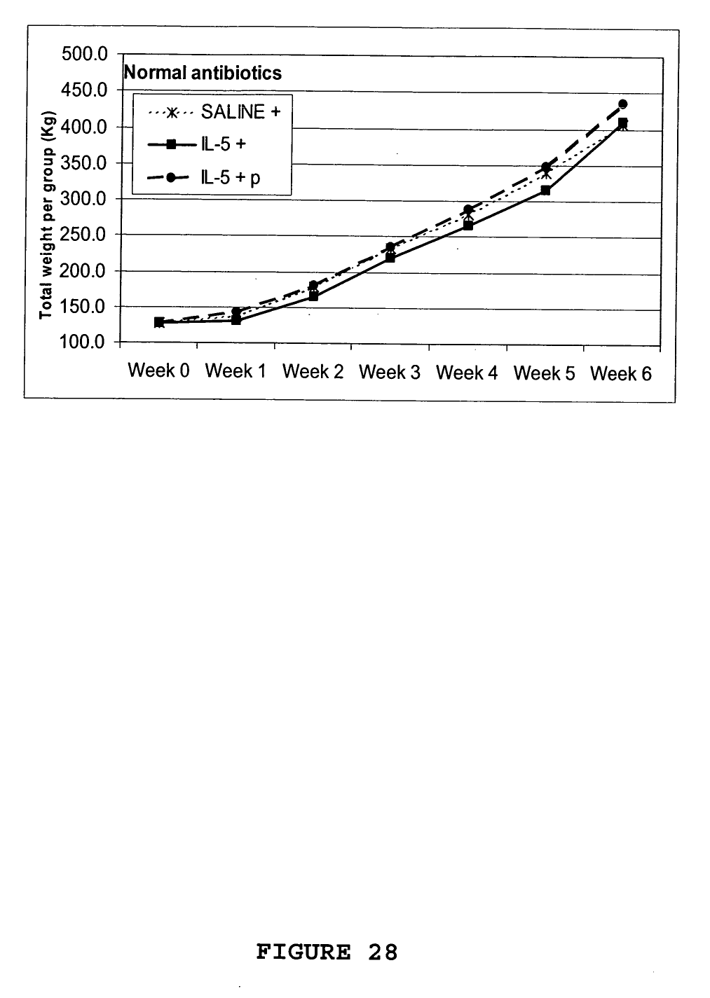

Unfortunately, a severe outbreak of post weaning scours affected all treatment groups. The most likely cause of the scours was E. coli. The effect of this infection(s) resulted in reduced weight gain as compared to the previous trial at the commercial piggery. FIG. 25 compares the average weights of the saline controls, with and without antibiotics, from this experiment and the previous experiment. This figure shows that the saline controls from this trial started with a higher average weight (0.7 kg) and finished at the end of the Weaner stage with a lower average weight for the saline medicated group (almost 2 kg lower). The infectious disease (scours) affected the un-medicated saline group at an earlier stage than the medicated group. The total weights of groups with the different antibiotic regimes are shown in FIGS. 26, 27 and 28.

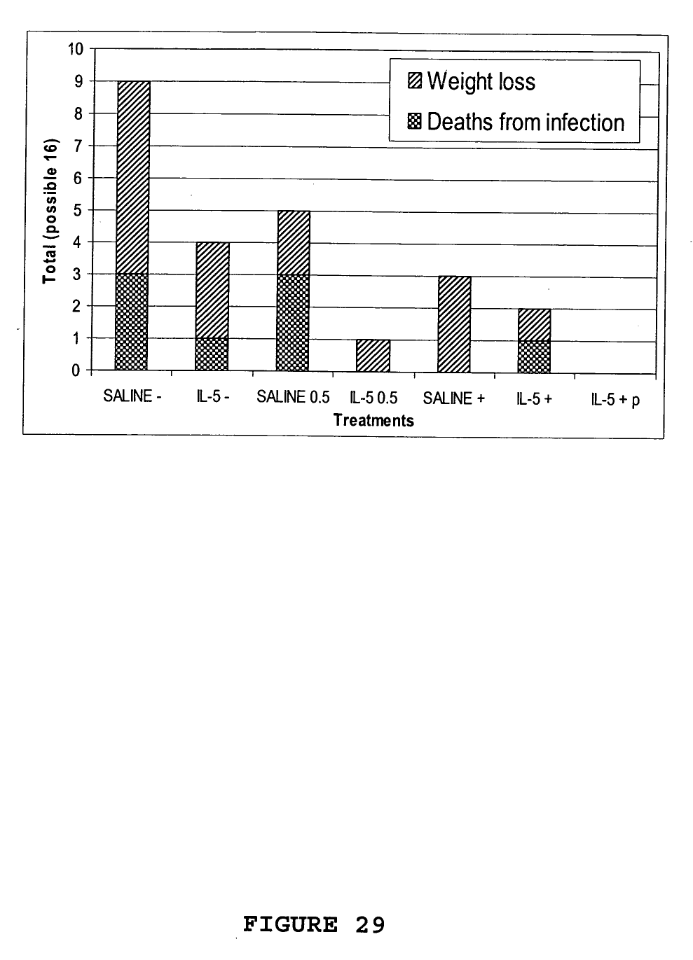

Weights were taken from the start to the end of the weaner period. These data represent differences in weight and number of pigs remaining in each treatment group. It can be seen from the results that IL-5 administration had beneficial effects on total weights, especially with reduced or no antibiotic supplements. These results show that IL-5 administration can increase the growth rate of pigs above controls in the face of infectious disease. This benefit was also reflected in the production loss as measured by deaths from infectious disease, or weight loss over any given week during the weaner period (FIG. 29).

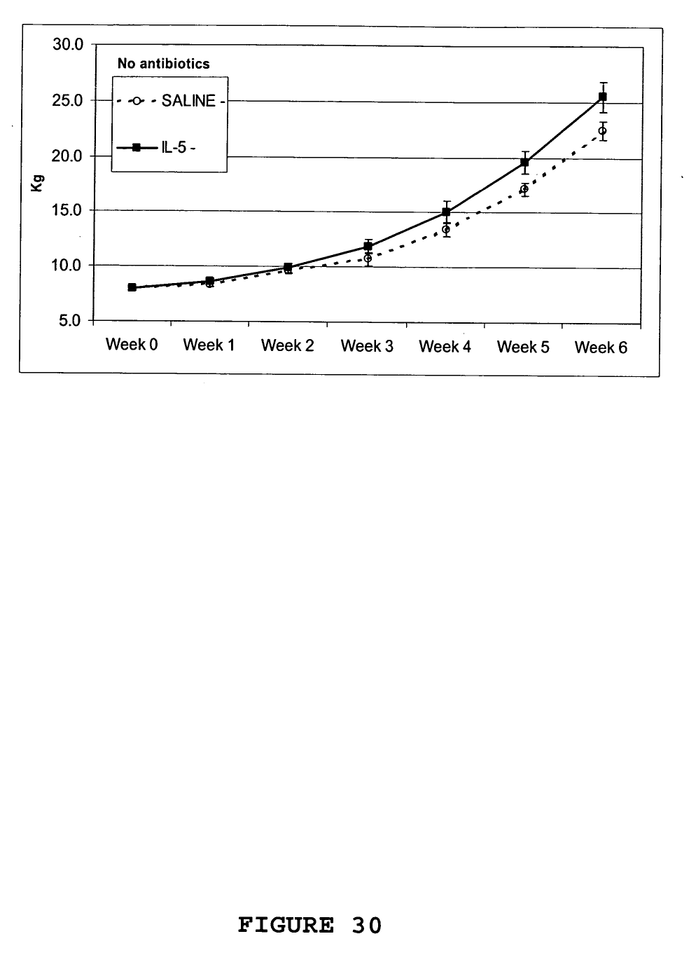

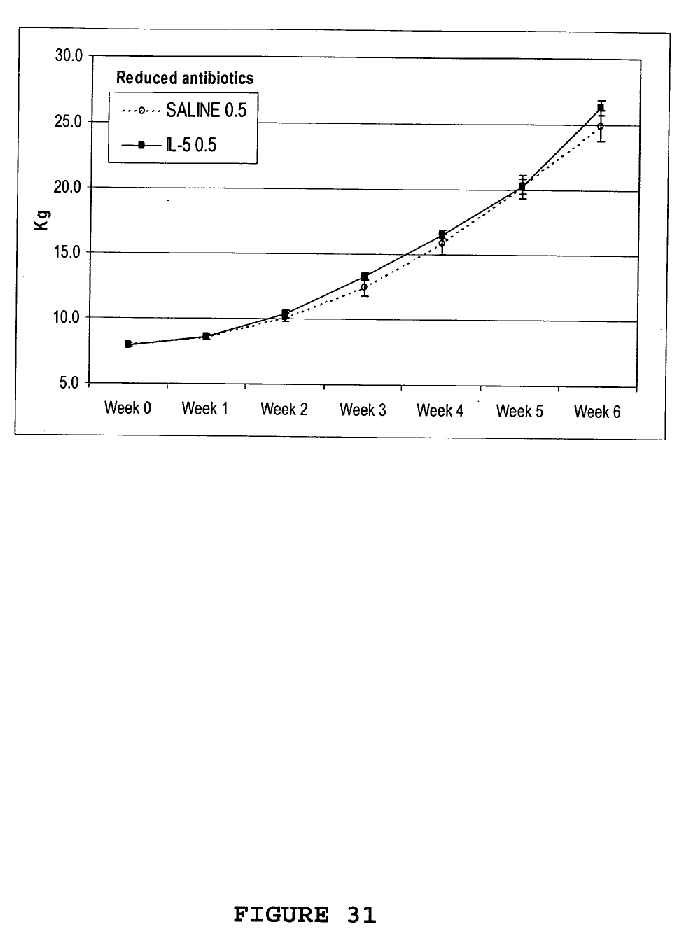

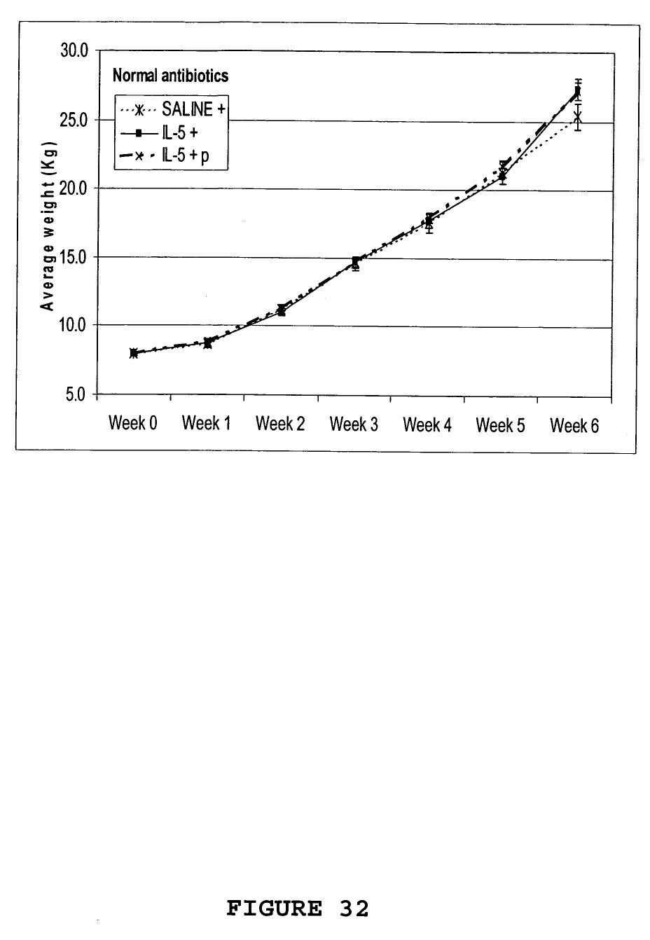

H. parasuis deaths occurred in IL-5+ (2 days into weaner period, presumably infected prior to the start of the trial). All other deaths were a result of scours. Weight loss was defined as one or more weekly weight reductions of individual pigs. IL-5 treatment groups had less production loss compared to their respective saline controls. Antibiotic supplements also lessened production loss. Weight loss could have also been a result of stress, especially at the time of weaning when pigs are removed from sows, transported, mixed in different social groups and fed dry food. Groups treated with IL-5 had consistently higher average weight gains during the Weaner period (FIG. 30) compared to the saline controls without antibiotic supplements in water or feed. The higher average weights were presumably due to reduced severity of disease and associated weight loss. Furthermore, IL-5 was shown to reduce the clinical effects of a haemolytic E. coli challenge. The pattern of higher average weight gains in IL-5 treated pigs was repeated with the reduced level and normal level of antibiotic supplementation (FIG. 31 and FIG. 32 respectively). However, the positive effect of IL-5 treatment was not as pronounced as that observed without antibiotic supplementation (FIG. 30).

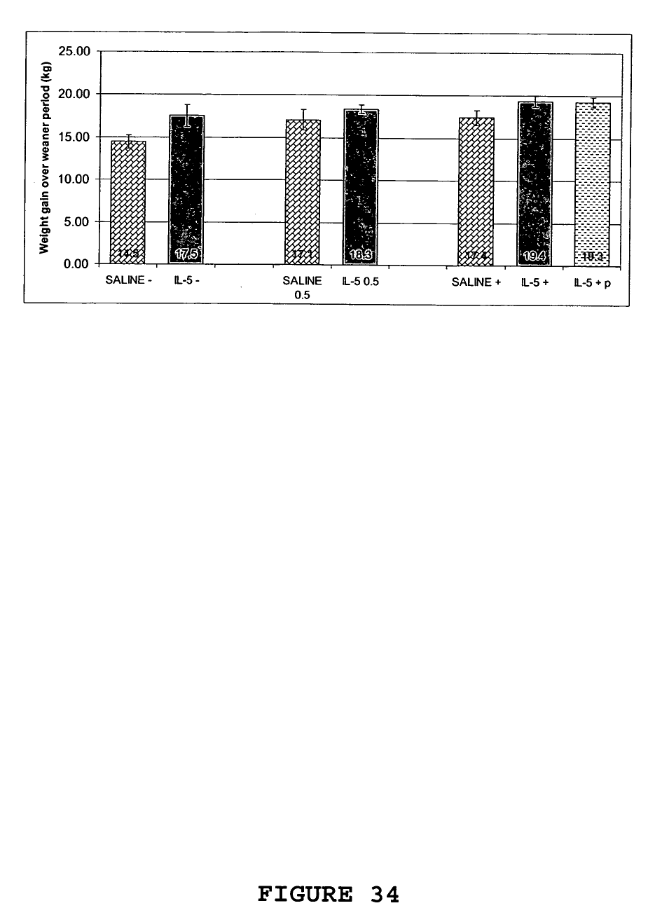

The average weight of each group throughout the weaner period is shown below in FIGS. 30, 31 and 32 and the average weight gained over the weaner period in FIG. 34. The IL-5 treated pigs showed higher average weights than the respective saline controls with each antibiotic regime at nearly all time points (FIGS. 30-32). The error bars (standard deviation/sqrt(number remaining) did not overlap for the cytokine and saline control for both the no and normal antibiotic regimes and was indicative of significant increases in average weights for the IL-5 treatments. Furthermore, the average weights of all cytokine treated groups were higher than all saline control groups demonstrating the beneficial effects of IL-5 on growth performance and/or health. There were no obvious or significant trends in average weights between males and females (data not shown).

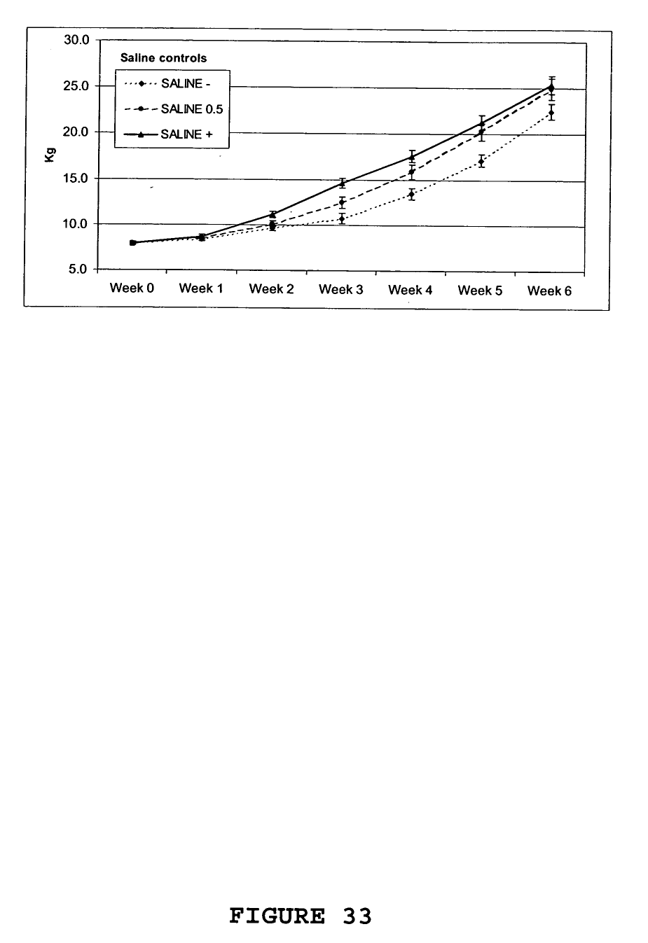

FIG. 33 shows the effects of three different antibiotic regimes on average weights of saline treated pigs throughout the trial. Antibiotic supplementation clearly increased the average weight gain in pigs in this trial and demonstrates the need for growth promotion and/or immune stimulation to increase health and productivity.

Pigs in groups treated with IL-5 had consistently higher average weight gains during the Weaner period compared to the respective saline controls with or without antibiotic supplements in water or feed (FIG. 34). These results also showed that antibiotic supplements result in consistently higher average weight gains during the Weaner period compared to the saline control without antibiotic supplements in water.

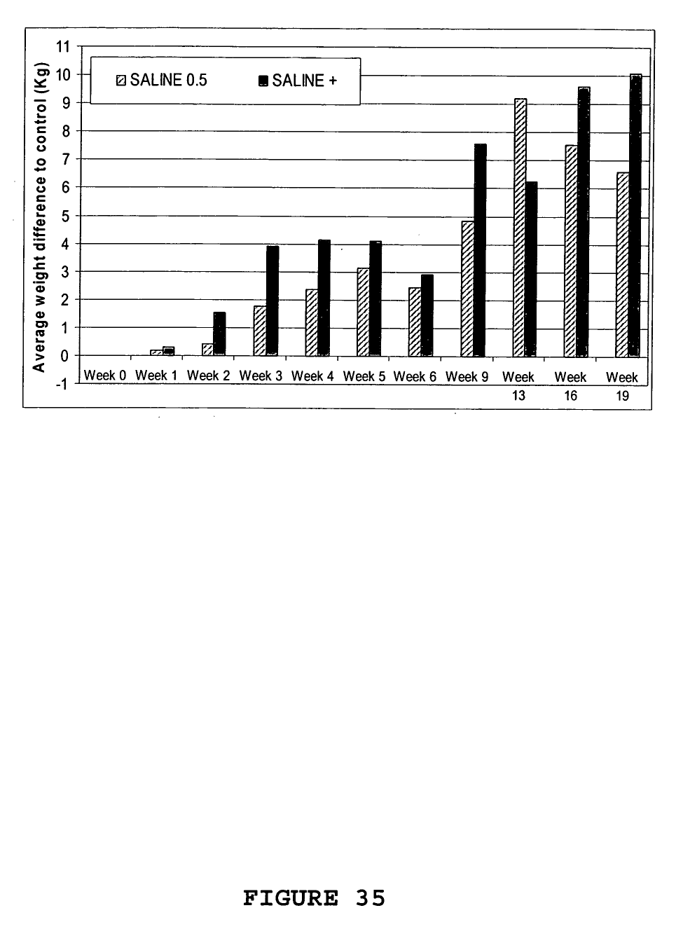

FIG. 35 shows the average weight gain over the weaner period compared to no-antibiotic saline control. The conclusions drawn were that antibiotic supplements resulted in consistently higher average weights than the saline control (without antibiotics) during weaner, grower and finisher periods. Also that reduced antibiotics increased the final average weight of 6.6 kg/pig (˜8%), while normal antibiotics resulted in an increased final average weight of 10.1 kg/pig (˜12%).

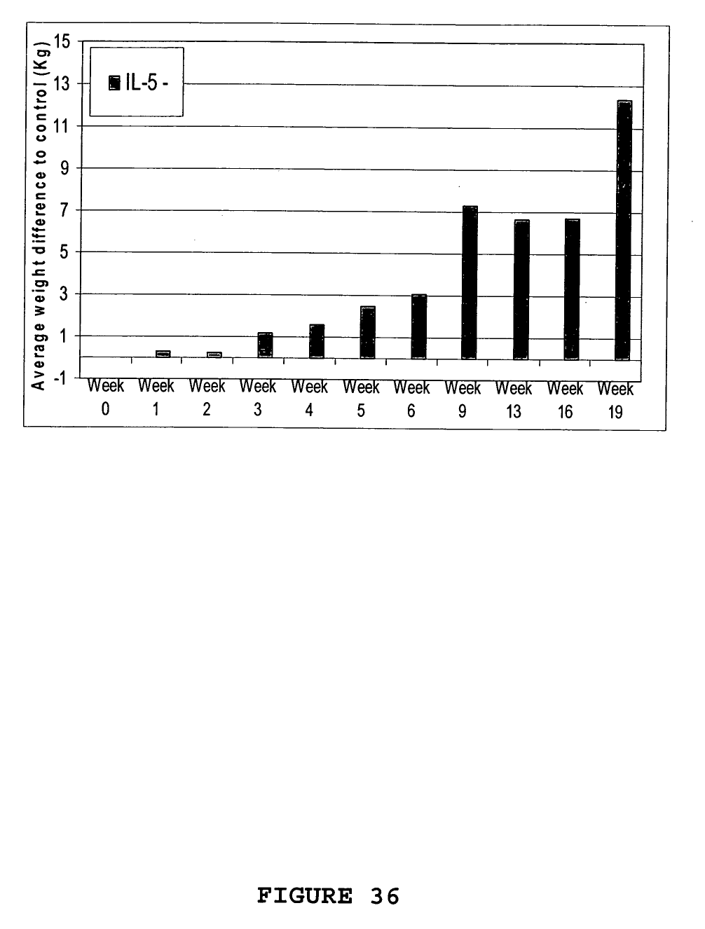

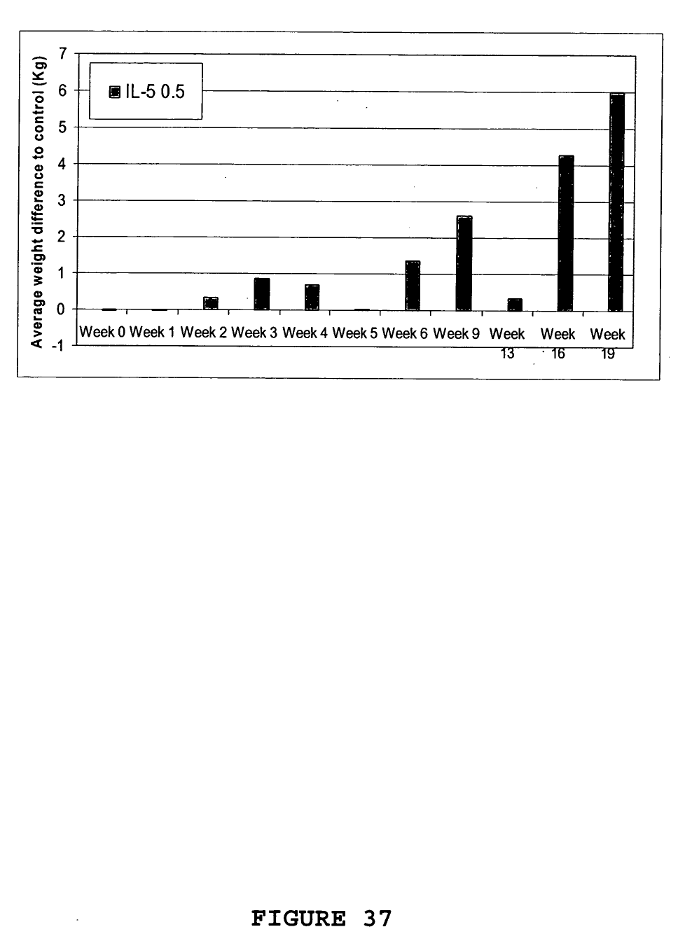

As shown in FIG. 36, IL-5 administration (without antibiotics) resulted in consistently higher average weights than the control during weaner, grower and finisher periods. IL-5 increased the final average weight of 12.3 kg (˜15%) over saline controls. The average weights compared to saline control with reduced antibiotics (Saline 0.5) are shown in FIG. 37. IL-5 administration (with reduced antibiotics) consistently resulted in higher average weights than the saline control (with reduced antibiotics) during weaner, grower and finisher periods. IL-5 also increased the final average weight of 6 kg (˜7%) over saline controls.

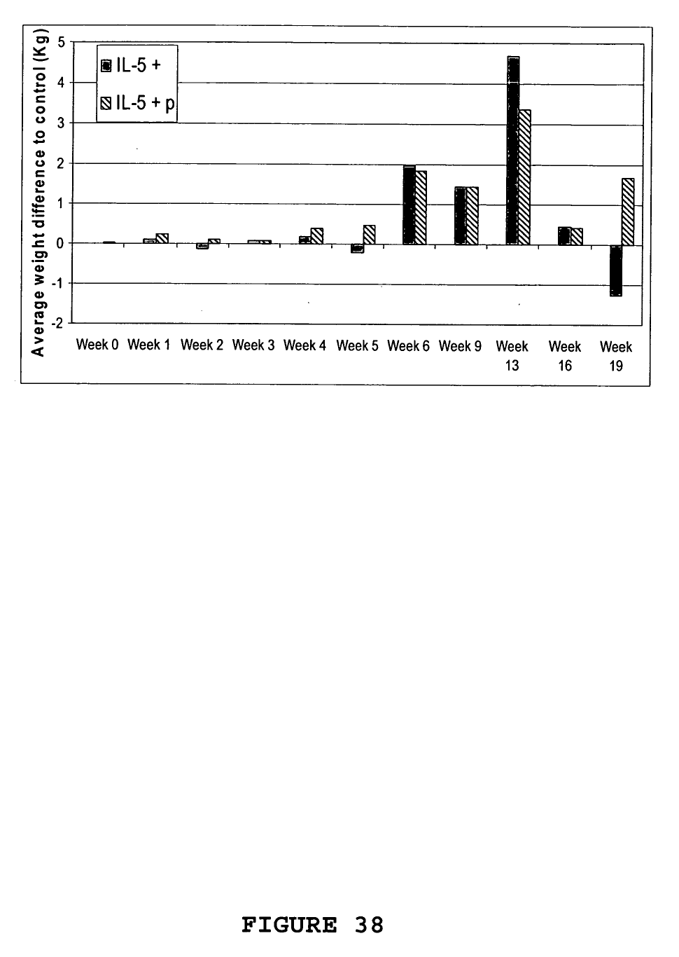

FIG. 38 shows the average weights compared to saline control with normal antibiotics (Saline+). IL-5 administration (with normal antibiotics) resulted in consistently higher average weights than the saline control (with normal antibiotics) from the end of the weaner period (W6) to the end of the grower period (W13).

IL-5+—was administered during W, G, F periods, whereas IL-5+p was administered during Weaner period only. IL-5+ decreased final average weight of 1.3 kg (˜−1.5%), while IL-5+p increased final average weight of 1.7 kg (˜2%). Consequently, pigs in the groups treated with IL-5 had consistently higher average weight gains during the Weaner, Grower and Finisher periods compared to the saline control without antibiotic supplements in water or feed. The results also showed that antibiotic supplements result in consistently higher average weight gains during the Weaner, Grower, and Finisher periods compared to the saline control without antibiotic supplements in water. IL-5 treatment was comparable to antibiotic supplements in terms of weight gain and reduced deaths from infectious disease. Within the normal antibiotic groups the saline control had reduced average weights compared to all cytokine treatments from the end of the weaner period to the end of the grower period. This trend did not continue during the finisher period, where the final average weights of all normal antibiotic groups were similar. The reason for this change was not known and was observed for the previous trial in a commercial piggery. This was highlighted by the 6 kg difference in average weights between IL-5+ and the saline control during the finisher period (IL-5+ was 4.7 kg higher at Week 13 and 1.3 kg lower at Week 19). This discrepancy was also highlighted by the difference between IL-5+ and IL-5+p (IL-5+was 1.3 kg higher at Week 13 and 2 kg lower at Week 19 compared to IL-5+p).

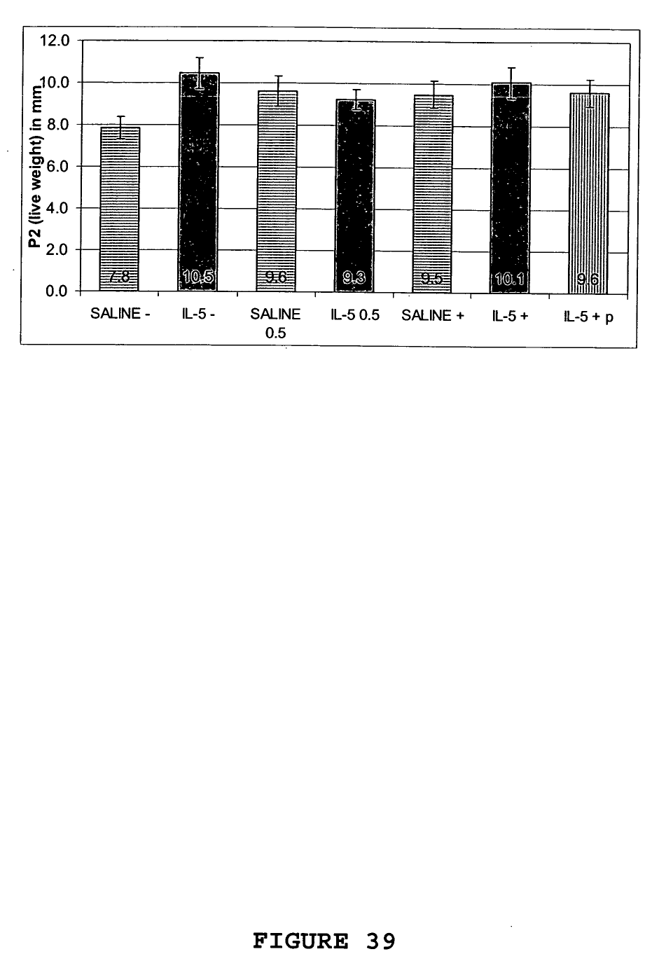

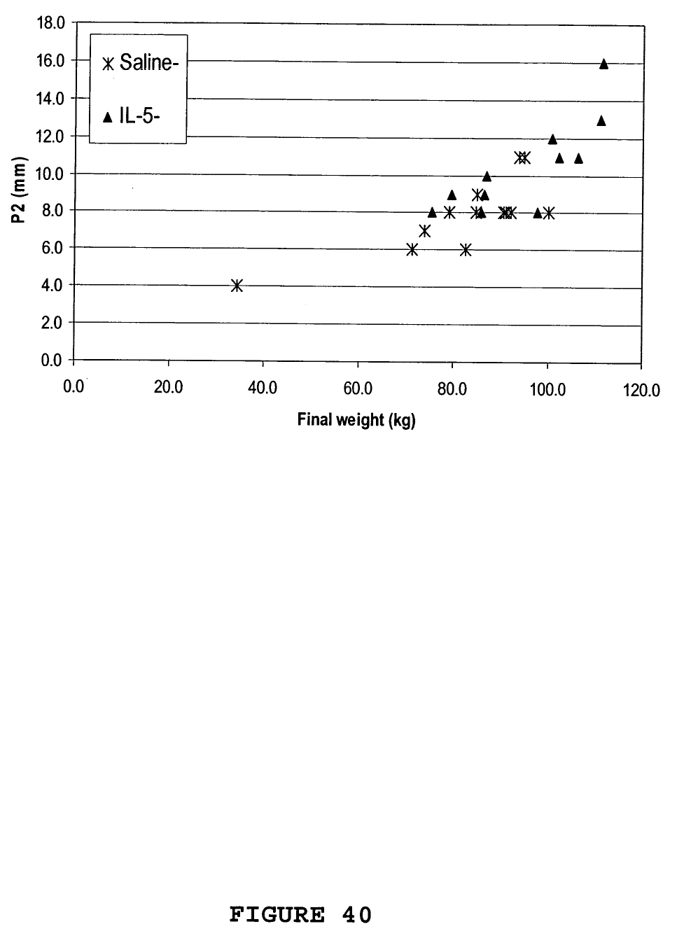

FIG. 39 shows that the cytokine treatments had similar backfat values as measured by P2 values, except for the unmedicated groups. A plot of P2 versus final weight for individual pigs showed the main difference between saline- and IL-5− groups was due to the lower individual weights of the saline-group (FIG. 40). FCR was measured in each period and no obvious differences were detected (data not shown).

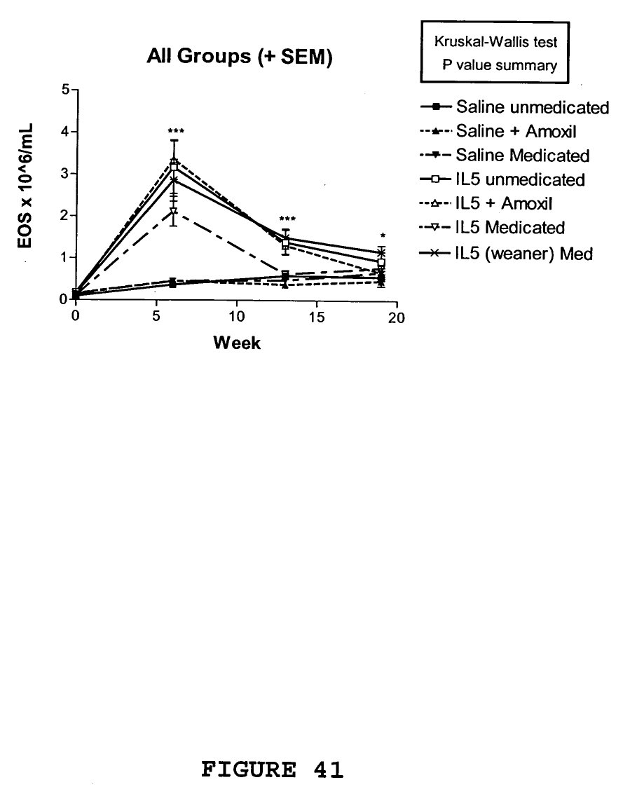

Eosinophil levels for all groups was also determined and these are shown in FIG. 41.

Haematology and differentials were undertaken at various time points during the trial. There were no significant changes between groups for these parameters except for eosinophil levels (FIG. 41). IL-5 significantly increased the levels of eosinophils in blood (both in terms of absolute numbers and differentials).

IL-5 administration substantially increased the average weights of pigs compared to saline controls. This was particularly evident in the groups without antibiotic supplements (IL-5: an increased final average weight of 12.3 kg or approximately 15%). Although there was a difference in the normal antibiotic groups between the saline control and the cytokine treatments in the weaner and grower periods, it did not continue through to the end of the finisher period. At the end of the grower period, normal antibiotic IL-5 treated pigs had increased average weights compared to the saline control. These increases in weight gain were substantial, but did not translate to increased weight gain at slaughter.

IL-5 appeared to reduce production loss during the weaner period in all antibiotics treatment groups. IL-5 was shown to reduce the detrimental effects of E. coli challenge. IL-5 may increase resistance to infection, especially with natural challenge, with or without antibiotic supplements in water or feed.

IL-5 appeared to protect pigs from infectious challenge and possibly have growth promoting effects in pigs without severe disease challenge. IL-5 administration also appeared to reduce the variation in weights or weight gain.

IL-5 reduced the effects of disease challenge and consistently increased average weights in the absence of antibiotics compared to un-medicated controls.

The IL-5 administration had substantial affects on average weight increases during the trial, and there was no significant differences between groups given IL-5 during the weaner or continuously throughout the weaner, grower and finisher periods.

### Summary of IL-5 Trials

Antibiotic supplements to feed and water at sub-therapeutic levels resulted in higher (>10%) average weight, weight gain or total weights during the weaner period (both trials).

The increased growth performance of antibiotic medicated groups at the end of the weaner period translated to increased (2-10%) processed productivity at slaughter (both trials) (also warm carcass weight, 1^{st} trial). This increase may have been greater if the antibiotic supplements were also withdrawn from the grower and finisher periods in the 1^{st} trial (all pigs were medicated during these periods). There were significant differences (P<0.001) between the non-antibiotic and normal antibiotic saline controls in terms of average weights at slaughter in the 2^{nd} trial.

IL-5 significantly increased circulating eosinophils in blood (both trials).

The beneficial effects of IL-5 plus medication on growth performance and health were outstanding in the 1^{st} trial. This was evident from the 18% increase in total weight, 9% increase in weight gain or 7% in average weight at the end of the treatment period compared to the saline medicated control group (1^{st} trial). This result was repeated in the 2^{nd} trial where IL-5 plus normal medication groups had an 11% increase in weight gain and a 7% increase in average weights over the respective medicated saline control (males and females used) during the weaner period.

Antibiotic supplements reduced the production loss in terms of weight loss of pigs during the weaner period (both trials). IL-5 also reduces production loss compared to respective saline controls (both trials).

There was severe disease challenge and 10-20% mortality in all groups except for the IL-5 medicated group (1^{st} trial). IL-5 also reduced the production loss in terms of weight loss and may have enhanced resistance to infection in the medicated group that was not evident for the un-medicated group. There was a severe post weaning scours in the 2^{nd} trial and full antibiotic supplements and/or IL-5 or IRAP reduced the production loss.

In addition to the increased growth performance and decreased production loss with IL-5 administration, there was reduced variability in size and growth of pigs during the weaner period when administered with IL-5. This trend continued for the IL-5 group through to final weight (D133). Although there was a similar trend in the 2^{nd} trial with IL-5 administration the reduced variability was not as significant.

IL-5 treated groups had-significantly higher average weights and average weight gain compared to the respective saline controls (2^{nd} trial). This was particularly important for reducing the antibiotic use or increasing the growth rate of pigs raised without antibiotic supplements. This was highlighted by the fact that all cytokine treated groups (with, without and reduced antibiotic regimes) have higher average weights and average weight gain than every saline control group ie. the IL-5 treatment groups without any antibiotic supplements have equivalent or higher average weights than the full medicated saline group.

One of the most relevant production parameters is the processed carcass weight (warm carcass weight—viscera, trotters and head). The antibiotic medicated saline pigs had an average warm carcass weight almost 3 kg higher than the un-medicated saline group. In contrast, the medicated IL-5 treated pigs increased the average warm carcass weight by 6 kg over the medicated saline control. All IL-5 treated groups had equivalent or higher average warm carcass weights compared to the saline un-medicated control. Various immune and haematology parameters were measured. Although the most obvious trend involved eosinophils with IL-5 administration all parameters and production traits were analysed for statistical difference by an independent source. Table 3 shows the number of deaths during trial (Days 42 and 133). Started with 20 per group.

TABLE 3
NUMBER OF DEATHS DURING TRIAL

Treatment:

| | Saline+ | IL-5+ | Saline− | IL-5− |
|---|---|---|---|---|
| Weaner: | 3 | 0 | 1 | 2 |
| Final: | 4 | 0 | 2 | 3 |

The conclusions drawn from Table 3 are that there were no deaths in the IL-5 medicated group during trial. Also there were deaths in every other group ranging from 10 to 20% by the end of the trial, with most deaths occurring during weaner period.

IL-5 medicated pigs and the IL-5 un-medicated pigs tended to have a more consistent weight range than other groups that is an economic benefit to some piggeries (FIG. 22). Only the IL-5 medicated groups had all individual weights above 90 kg (note that the IL-5 medicated group had no deaths and that deaths in the other groups generally involved lower weight pigs).

As shown in FIG. 23, the IL-5 treated pigs had significantly higher % dressing than the respective saline controls (p, 0.045). The pigs from the medicated groups have higher % dressing than the un-medicated groups. The IL-5 medicated pigs have a substantially higher warm carcass weight than the saline medicated control. The medicated groups have higher warm carcass weights than the un-medicated groups (FIG. 24).

### EXAMPLE 6

### Delivery of Recombinant IL-5 to Weaner Pigs Infected with Haemorrhagic E. coli

This study determined whether IL-5 was able to improve the health of pigs exposed to infections, such as haemorrhagic E. coli.

One aim was to determine whether IL-5 could improve growth in pigs infected with E. coli at weaning. A further aim was to determine whether IL-5 could reduce infection rates and improve health in pigs infected with E. coli. Finally, it was hoped that an assessment of the prophylactic or therapeutic potential of IL-5 against E. coli infections in weaner pigs could be determined relative to current antibiotic treatments.

Male weaner pigs, with a mean weight of 5.4 kg were allocated to groups of 8, with the mean weight being equalised between groups. Pigs were housed in group pens. Pigs were provided with pelleted feed and water ad libitum.

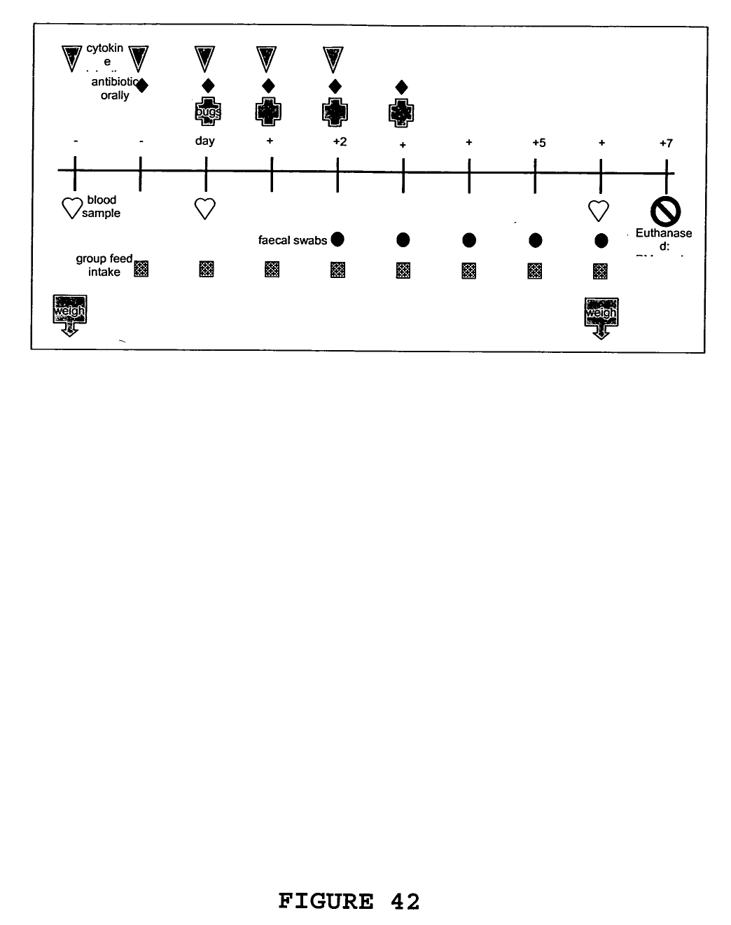

Pigs were treated with cytokines or the antibiotic, Apralan, and challenged with E. coli according to the schedule outlined in FIG. 42. E. coli were delivered orally in an 8 ml dose containing 10⁸ cfu/ml. Blood was sampled from pigs by venipuncture at −2 days, day 0, and +6 days from initial challenge with E. coli as outlined in FIG. 42. Blood was assayed for immunological parameters as previously described. Pigs were weighed at day −2 and at the end of the trial on day 7.

Faecal samples were taken from each pig daily from day 2 to day 6 after challenge; these samples were cultured on sheep blood agar to quantify E. coli load. The condition of faeces on each day of challenge was noted as normal, wet or diarrhoea, as an indication of clinical signs.

At the conclusion of the experiment, pigs were euthanased and samples were taken from different areas in the gastro-intestinal tract, including the small intestine (25%, 50% and 75% along the length of the small intestine), the caecum and colon, and from the faeces. These post-mortem samples were also plated out on sheep blood agar to quantify E. coli load. Growth on sheep blood agar was scored from 0 to 5 (where 0 was no growth, 1 signified growth in the primary inoculum, 2 signified growth in the first streak, 3 signified growth in the 2^{nd} streak, 4 signified growth in the 3^{rd} streak, and 5 signified growth of E. coli in the final streak), and group means and standard errors were calculated.

Table 4 shows the treatments and doses applied in the cytokine experiment.

TABLE 4
TREATMENTS AND DOSES APPLIED
IN CYTOKINE EXPERIMENT
(N = 8 PER GROUP)

| Treatment | Treatment Dose |
|---|---|
| saline | 1 ml |
| IL-5 | 200 μg in 1 ml |
| Apralan | 12 mg/kg in 2 ml |

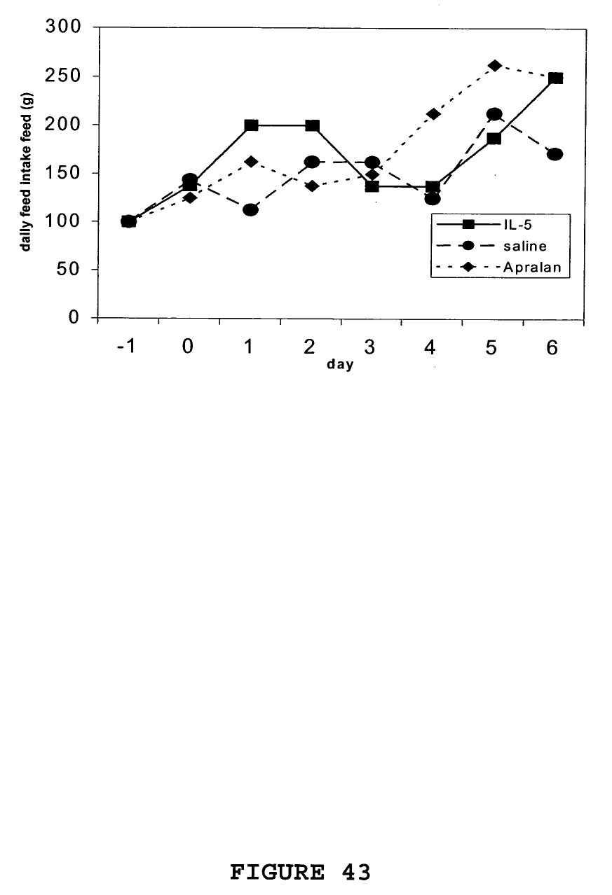

FIG. 42 shows the time line sequence of events for the cytokine experiment with E. coli challenge. It can be seen that the pigs that were treated with IL-5 or Apralan improved appetite compared to saline treated pigs (FIG. 43). This improvement in intake did not alter feed conversion efficiency (data not shown). Increased appetite was indicative of improved health and reduced inflammatory responses. Due to the short duration of the challenge, there was no significant difference between treatment groups for weight gain over the 5 day challenge period.

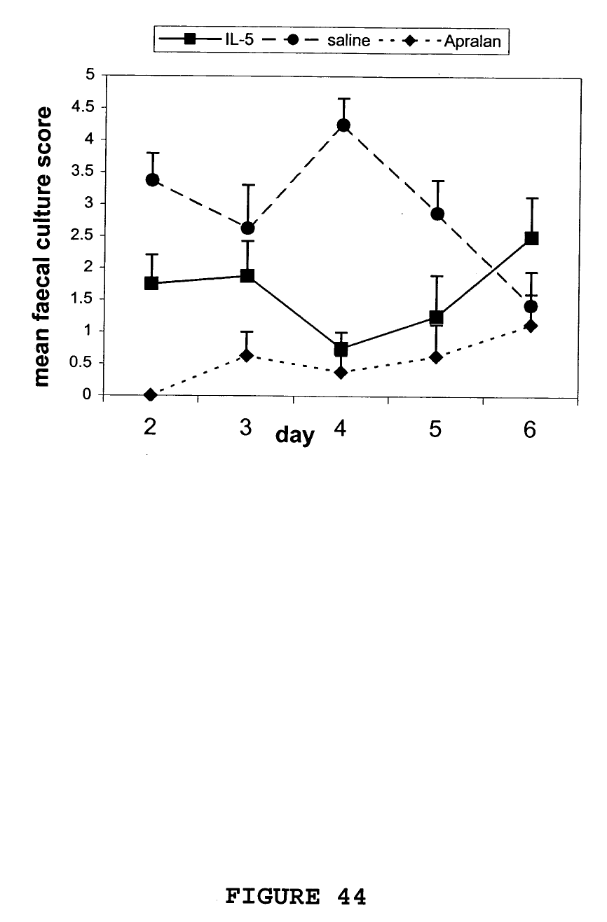

Pigs treated with IL-5 and Apralan showed decreased E. coli shedding in faeces compared to control pigs treated with saline (FIG. 44). Pigs treated with Apralan or IL-5 had reduced bacterial shedding from day 2 to day 5. On day 6 after challenge, bacterial shedding from all groups was equal. Overall, the Apralan treated group displayed the least bacterial shedding of all treatments.

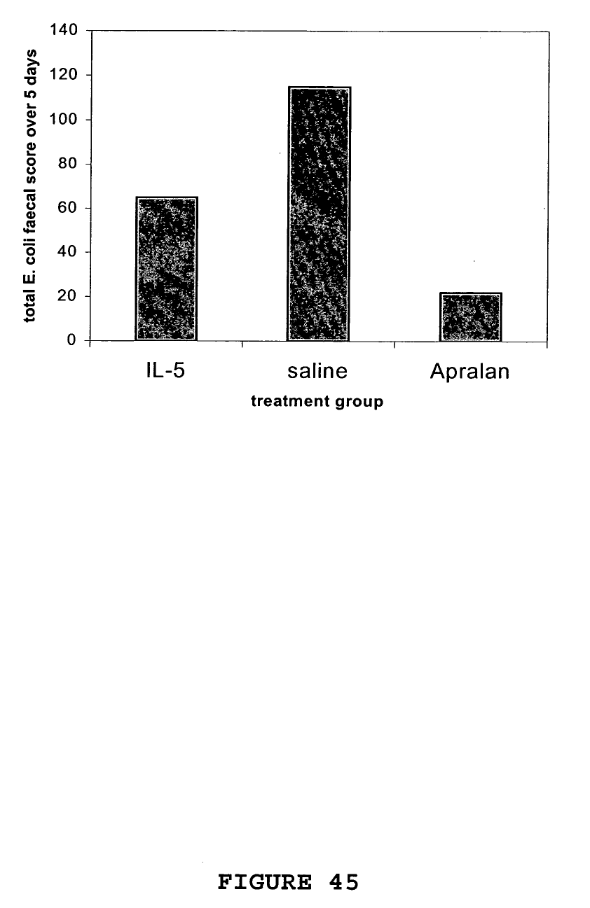

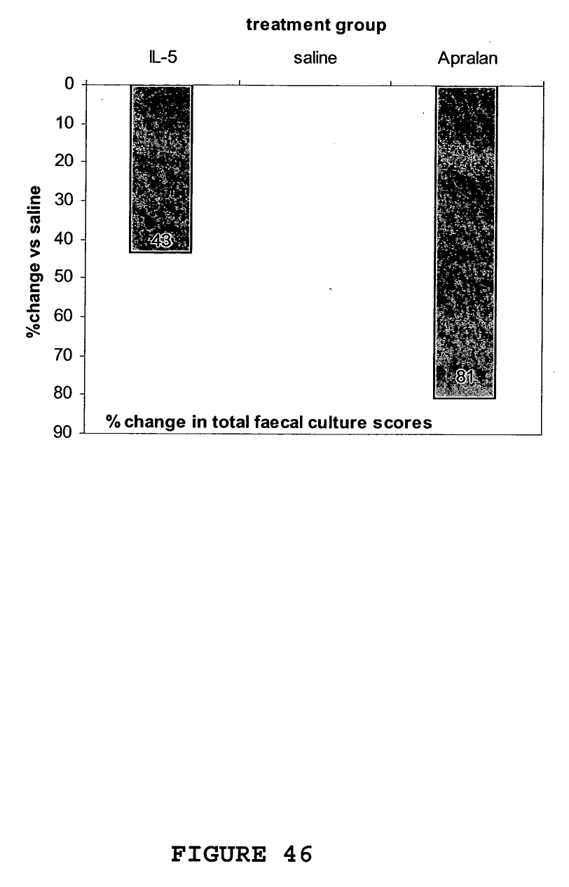

Faecal scores tallied over the entire challenge period for each group show an 80% decrease in faecal shedding for Apralan treated pigs compared to saline treated controls, while IL-5 treated pigs showed a 43% reduction in bacterial shedding compared to saline treated controls (FIGS. 45 and 46).

In commercial situations, reduced bacterial shedding from infected pigs would further reduce re-infection in other members of the herd or pen, thereby improving the health of weaners, and enhancing growth potential in later phases.

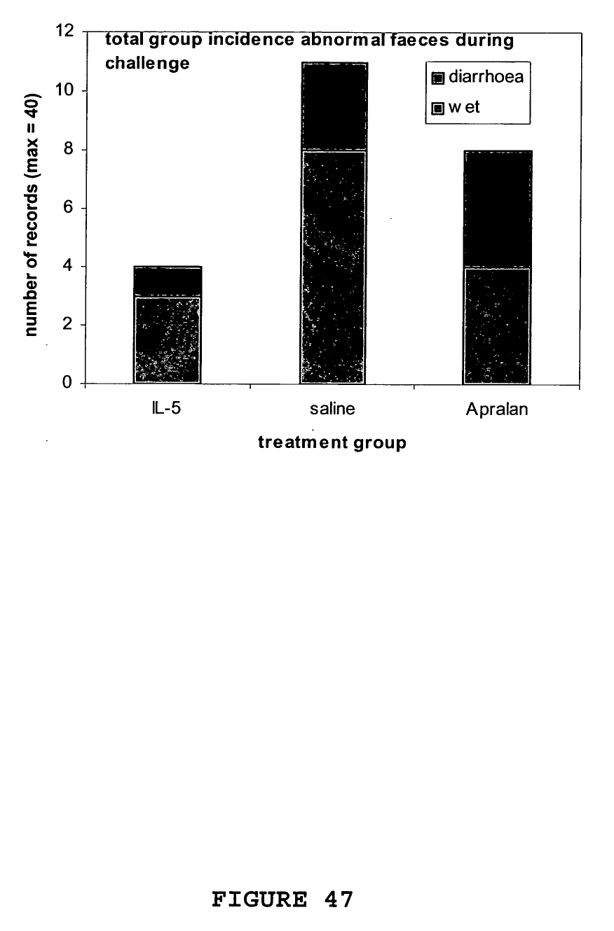

Clinical signs, recorded as the presence of wet faeces or diarrhoea, were decreased in pigs treated with IL-5 or Apralan (FIG. 47). Pigs treated with IL-5 had fewer recorded cases of wet faeces and diarrhoea than saline controls or Apralan treatment. Pigs treated with Apralan had less recordings of wet faeces than did saline controls, but also displayed a minor increase in the prevalence of diarrhoea in the post-challenge period (FIG. 47).

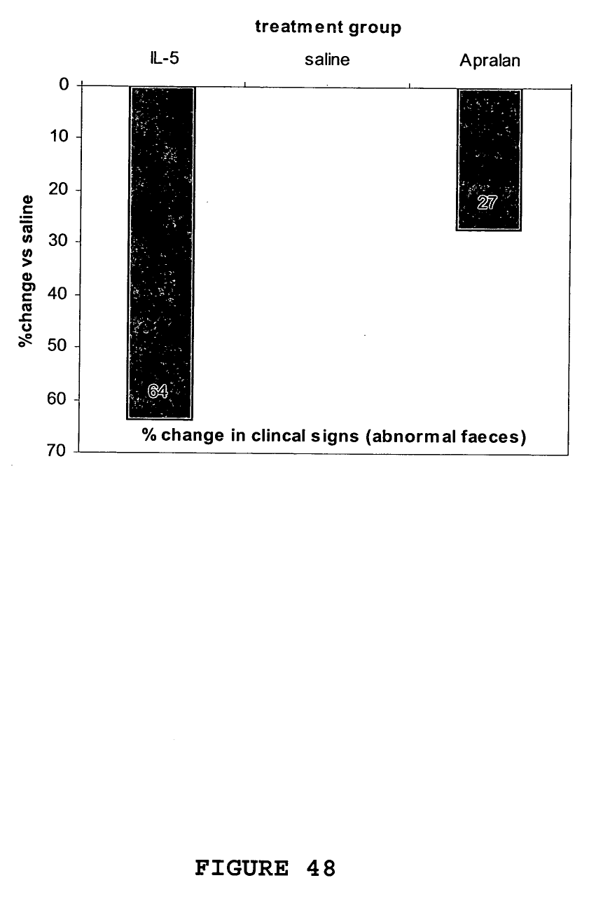

When these clinical signs were described as a percentage reduction in symptoms compared to saline controls, we found that the IL-5 treatment produced a 64% reduction in clinical signs, while Apralan caused clinical symptoms to be reduced by 27% (FIG. 48).

The results for clinical symptoms showed that IL-5 and Apralan were both able to reduce the outward signs of infection with E. coli. In this measure of health, IL-5 performed as well as Apralan, the current antibiotic treatment for E. coli infections.

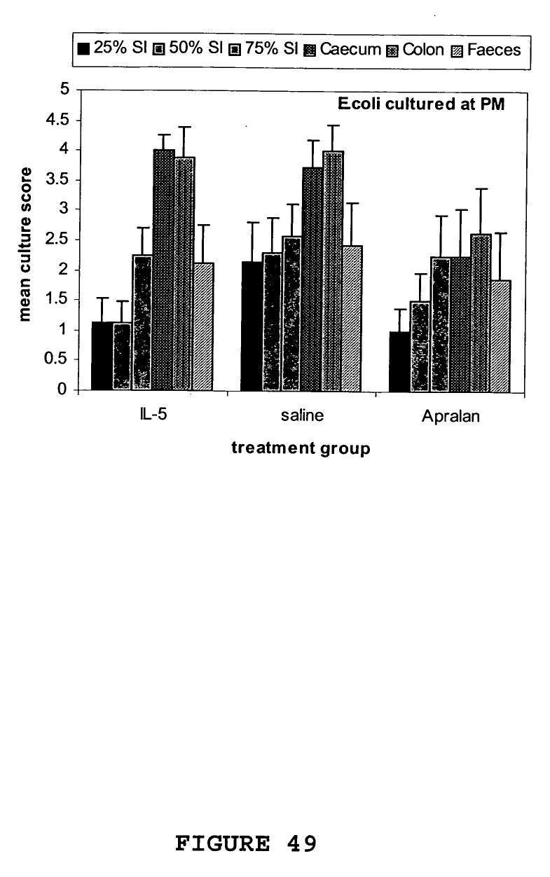

Both Apralan and IL-5 treatments resulted in reduced bacterial load in most areas of the gastro-intestinal tract (GIT) compared with saline-treated controls (FIG. 49). The effect of IL-5 treatment was most noticeable in the small intestine.

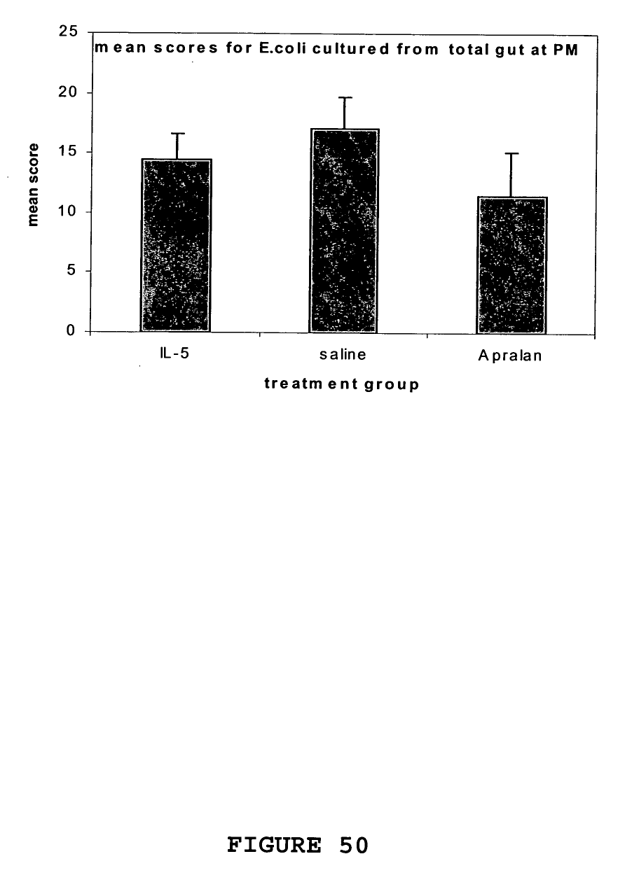

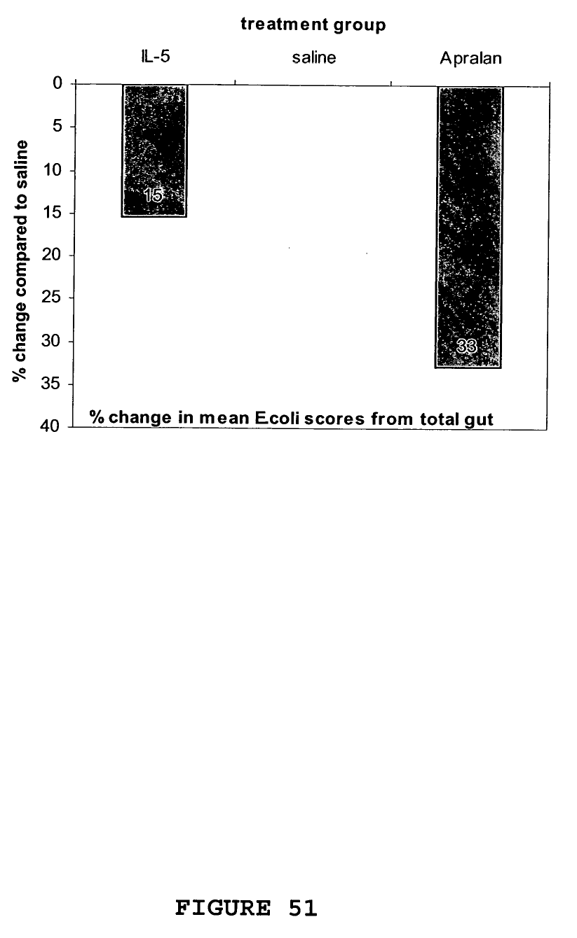

When all culture scores were tallied for each pig and used to calculate group mean total scores (FIG. 50), pigs treated with IL-5 scored less than 15 out of a possible 30, compared with 17/30 for saline treated pigs, and 12/30 for Apralan treated pigs. When these data were expressed as a percentage reduction in E. coli culture scores compared to saline controls (FIG. 51), prophylactic application of IL-5 resulted in a 15% reduction in the amount of E. coli in the gastro-intestinal tract.

These results illustrated that bacterial load was reduced in pigs treated with IL-5 when compared with saline controls, further emphasising the value of this preparation for the control of haemorrhagic E. coli in young pigs.

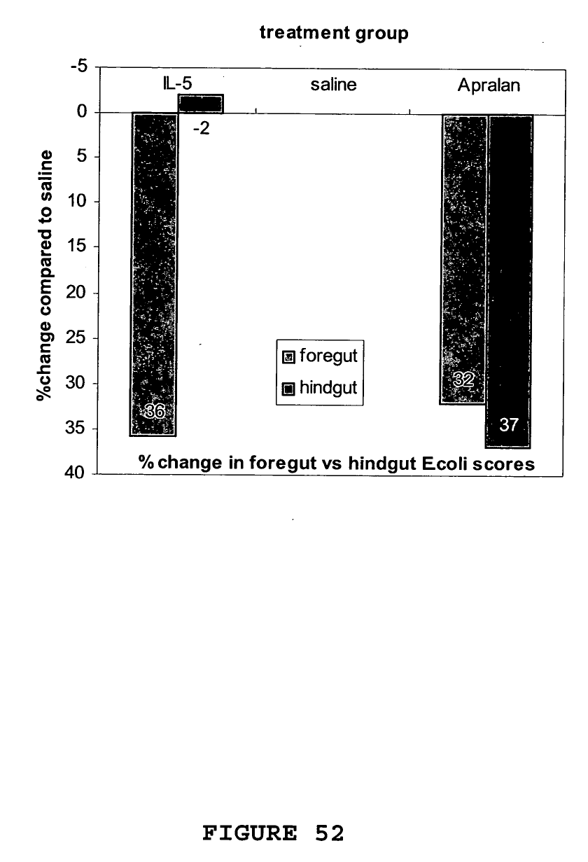

When the post-mortem results for E. coli cultures were separated on the basis of location in the gut, differences may be seen in the action of IL-5 and Apralan (FIG. 52). E. coli bacterial load in the small intestine (foregut) correlates with the severity of disease, as the small intestine is the site in which the secretory diarrhoea is manifested. Treatment with IL-5 reduced the bacterial load in the small intestine by 36% compared with saline controls, while Apralan caused a reduction of 32% in the bacterial load in the small intestine. In the hindgut area (caecum and colon), bacterial loads recorded for Apralan were lowest of all treatments (FIG. 49). The ability of IL-5 to reduce bacterial load in the foregut suggested that the treatment might reduce the severity of disease associated with haemorrhagic E. coli infection. Thus, IL-5 might be a potential replacement or adjunct for the antibiotics currently administered in the pig industry to control the deleterious effects of this disease on pig production.

### Conclusions

IL-5 improved the health of pigs ie. it reduced the clinical signs of disease, in terms of faecal changes associated with haemorrhagic diarrhoea in the presence of haemorrhagic E. coli infection. It also improved appetite during challenge.

The improvement in health produced by IL-5 treatment was in some cases greater than that produced by treatment with the antibiotic Apralan, the current method of treating haemorrhagic E. coli in pigs.

IL-5 treatment resulted in decreased bacterial shedding in faeces during the course of infection compared with saline-treated controls. Pigs treated with IL-5 showed bacterial shedding significantly less than saline treated controls on 3/5 days after challenge. Such results suggested that under commercial conditions, infection rates might be reduced by decreasing the bacterial load in the environment.

The effect of IL-5 administration resulted in decreased numbers of bacteria in most areas of the GIT compared with saline treated controls.

Significantly, IL-5 caused a 36% reduction in the bacterial load in the small intestine (foregut), the site in which secretory diarrhoea is normally located during the course of E. coli infection. As bacterial load in the small intestine is associated with disease severity, IL-5 may have a significant therapeutic effect on the progression and pathology of the disease.

IL-5 treatment performed as well as Apralan, the current antibiotic treatment used in industry, in reducing clinical signs of disease, E. coli levels present in the gut at post-mortem, in addition to E. coli present in the crucial site of the small intestine.

A summary of the comparative effects of IL-5 and Apralan on bacterial shedding, clinical signs and bacterial load at post-mortem is included in Table 5.

### Summary Comparing the Therapeutic Effects of IL-5 (IL-5) and Apralan for the Control of Haemorrhagic E. coli Infections in Young Weaner Pigs. Dark Arrows Show Positive Effects, while Light Arrows Show Negative Effects in this Example

| Change compared to saline | | |
|---|---|---|
| treated controls | Apralan | IL-5 |
| Presence of bacterial shedding in faeces | 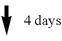 | 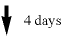 |
| Change in faecal bacterial load | 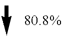 | 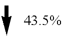 |
| Change in clinical signs | 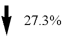 | 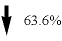 |
| E. coli at postmortem | 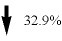 | 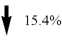 |
| E. coli in foregut | 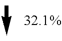 | 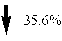 |
| E. coli in hindgut | 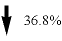 | 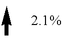 |

### EXAMPLE 7

### Delivery of recombinant IL-5 as a Prophylactic to Pigs Exposed to Swine Dysentery Challenge

One aim of this example was to determine whether IL-5 could improve the health of pigs infected with an enteric inflammatory pathogen causing swine dysentery, Brachyspira (Serpulina) hyodysenteriae. A further aim was to determine whether IL-5 could improve the growth rate of pigs under conditions of challenge with swine dysentery.

Male pigs with a mean starting weight of 6.5 kg, were allocated to treatment groups consisting of eight pigs (Table 6). Pigs were housed in group pens, with each pen containing a replicate from each of the treatment groups. One group of 8 pigs was housed in a separate room and left uninfected to act as untreated controls. Pigs were provided with pelleted feed and water ad libitum.

Prior to swine dysentery challenge, pigs were treated with recombinant IL-5 or saline, as described in Table 6. Cytokines and the antibiotic, Lincomycin, were delivered by intramuscular injection at intervals outlined in Table 7. Pigs were infected with Brachyspira hyodysenteriae at day 0, day 1 and day 2, given as an oral bolus of 120 ml of spirochaete culture in log phase of growth, containing approximately 108 cells.

Faecal swabs and blood samples were taken from each pig at intervals described in Table 7. Faecal swabs were cultured for the presence of spirochaetes. Blood samples were assayed for immunological parameters as described in example 1 above. Pigs were weighed at weekly intervals throughout the experiment, which was terminated by euthanasia on days 19 and 20 after the initial challenge. At post mortem swabs from areas of the hindgut were cultured for the presence of spirochaetes, and the gross pathological condition of the gastro-intestinal tissue noted.

TABLE 6
SUMMARY OF TREATMENT GROUPS FOR
CHALLENGE TRIAL TO ASSESS THE EFFICACY
OF IL-5 AS A PROPHYLACTIC TREATMENT FOR SWINE
DYSENTERY INFECTION (N = 8 PER GROUP)

| Treatment | Treatment Dose |
|---|---|
| saline | 1 ml |
| IL-5 | 1 ml @200 μg/ml |
| Lincocin | 2 ml (as per |
| | manufacturers |
| | instructions) |
| untreated | No treatment, no |
| | challenge |

TABLE 7
PROTOCOL FOR EXPERIMENTAL PROCEDURES TO ASSESS
THE EFFICACY OF IL-5 AS A PROPHYLACTIC TREATMENT
FOR SWINE DYSENTERY INFECTION

| | | | | | Inject 1 ml | Inject | |
|---|---|---|---|---|---|---|---|
| | | Faecal | | | IL-5, or | 2 ml | |
| Day | Weigh | Swabs | Infect | Bleed | Saline | Lincocin | Kill |
| −7 | X | | | | | | |
| −6 | | | | | | | |
| −5 | | | | | | | |
| −4 | | | | | | | |
| −3 | | | | | | | |
| −2 | | | | | X | | |
| −1 | | | | | X | | |
| 0 | X | X | X | X | X | | |
| 1 | | | X | | X | X | |
| 2 | | | X | | X | X | |
| 3 | | | | | | X | |
| 4 | | | | | | | |
| 5 | | X | | X | X | X | |
| 6 | | | | | | X | |
| 7 | X | X | | | X | X | |
| 8 | | | | | | X | |
| 9 | | X | | | X | X | |
| 10 | | | | | | | |
| 11 | | | | | | | |
| 12 | | X | | X | X | X | |
| 13 | | | | | | X | |
| 14 | X | X | | | X | X | |
| 15 | | | | | | X | |
| 16 | | X | | | X | X | |
| 17 | | | | | | | |
| 18 | | | | | X | X | |
| 19 | X | | | | | | X |
| 20 | X | | | | | | x |

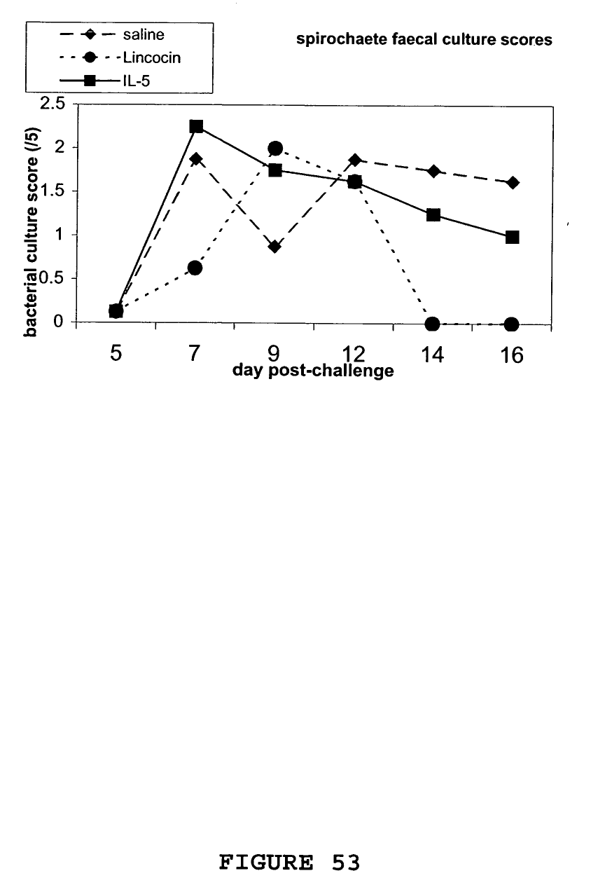

All groups of pigs infected with swine dysentery were shedding spirochaetes in faeces from day 5 after challenge as detected by faecal culture (FIG. 53). Pigs treated with IL-5 showed decreasing levels of spirochaete shedding by day 14 after challenge, compared to the saline treated control group, suggesting that IL-5 treated animals were resolving their infection more quickly than saline treated controls. Pigs treated with the antibiotic Lincocin showed no signs of faecal spirochaete shedding by day 14.

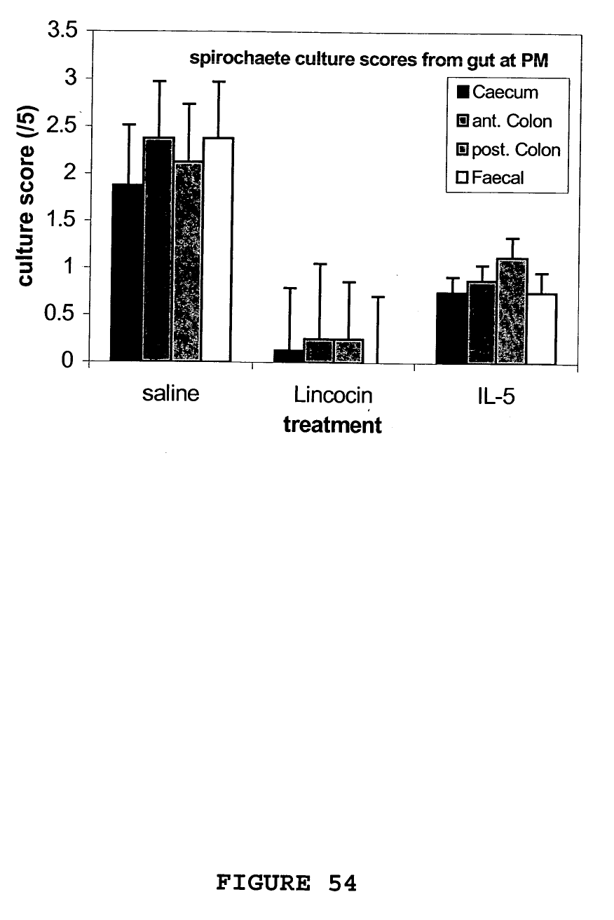

Spirochaete cultures taken from the hindgut at post mortem show that treatment of pigs with IL-5 reduced the number of spirochaetes residing in the gut compared to saline controls (FIG. 54). IL-5 was able to reduce spirochaete culture scores in the caecum, anterior colon, posterior colon and faeces compared to saline treated controls. Importantly, the effect of IL-5 in reducing spirochaete load at post mortem was comparable to that shown by the Lincocin antibiotic. Although achieving a similar result as Lincocin for spirochaete load, IL-5 treatment resulted in reduced variation, which implies that more consistent treatment results are possible with IL-5 application.

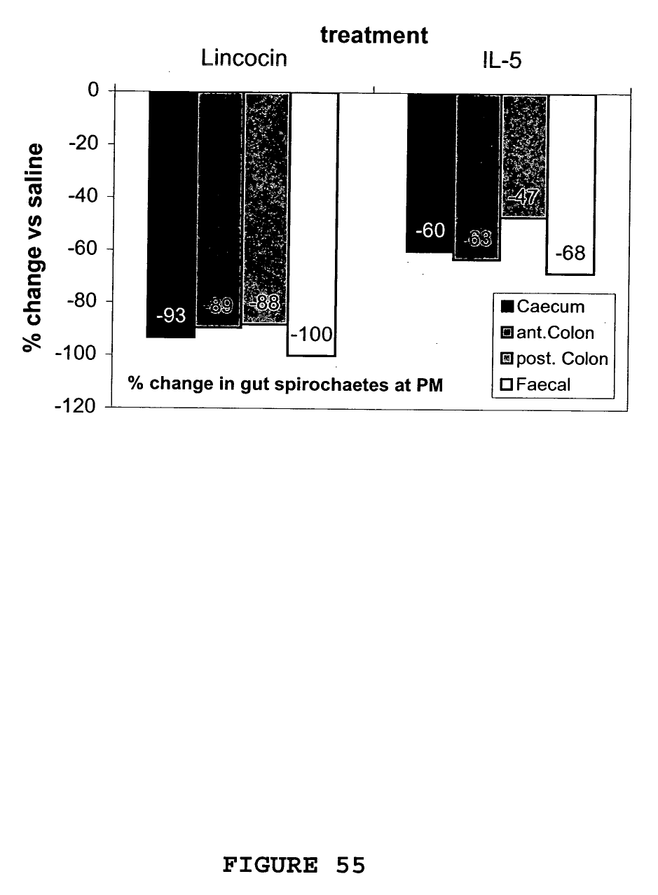

Compared to saline treated pigs, IL-5 treatment resulted in a 60% reduction in the number of spirochaetes in the caecum, 63% reduction in the anterior colon, 47% in the posterior colon and 68% reduction in faecal spirochaetes (FIG. 55). Lincocin treatment produced respective reductions of 93%, 89%, 88% and 100% for spirochaete load at post mortem.

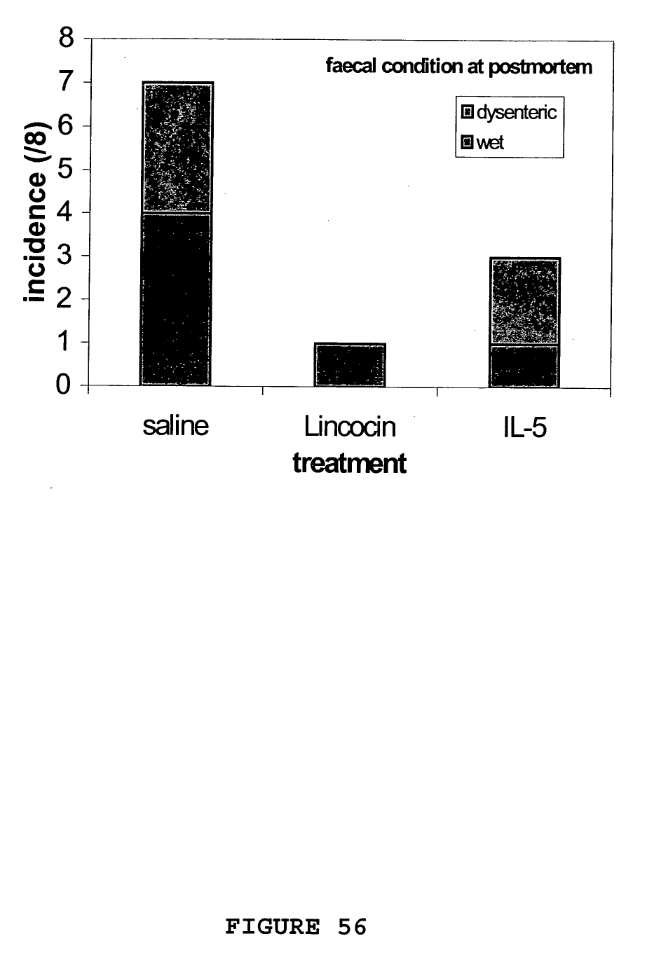

In addition to a reduction in the number of spirochaetes in the gut, treatment with IL-5 also reduced the clinical signs associated with infection indicated by faecal condition. FIG. 56 shows that IL-5 treated pigs showed fewer signs of dysentery-affected faeces (wet and mucoid with blood) or wet faeces (abnormally wet faeces unable to hold form) compared to saline treated controls. Of the 8 saline treated pigs, 7 showed clinical manifestation of swine dysentery determined by faecal condition, 3 of which were dysenteric mucoid and bloody in nature. Treatment with IL-5 reduced the incidence of clinical signs in faeces to 3 from 8 pigs infected (FIG. 56). Pigs treated with the Lincomycin antibiotic had negligible clinical signs of infection with only one of the 8 pigs in this group showing wet faeces, which was a comparable result with the uninfected control pigs.

Treatment of pigs with IL-5 reduced the number of spirochaetes present in the hindgut and faeces at post mortem compared with saline treatment. IL-5 reduced the clinical manifestation of swine dysentery infection as detected by faecal condition, compared with saline controls. Improved health, as determined by improved faecal condition, and reduced presence of spirochaetes in the gut and faeces at post mortem with IL-5 treatment, was comparable to the results obtained using the antibiotic Lincocin, the current therapeutic for swine dysentery infection in pig herds.

### EXAMPLE 8

### Administration of IL-3 to Pigs

This trial evaluated the capacity of IL-3 to improve growth performance and immunity of pigs by comparing the growth rate and health of weaner pigs (28 day old weaners is Day 0 of the trial and the weaner period continued for 42 days) through to the finisher stage (Days 93 to 113) and slaughter (133 days after commencing the trial), which were administered with the recombinant porcine cytokine, IL-3, and saline was used as a control, with and without standard weaner medicated water and feed in a commercial piggery environment.

Experimental Design

| Treatments | Administered |
|---|---|
| Saline | injection, needle IM neck muscle, |
| | 1 ml twice weekly for 6 weeks. |
| 100 μg IL-3 | (in saline) injection, needle IM |
| | neck muscle, twice weekly for 6 |
| | weeks of 100 μg of IL-3 in 1 ml |
| | saline. |

40 pigs per treatment were mixed in groups, with 4 replicates containing standard medicated water and feed and 4 replicates without medicated feed or water.

Overall weights for each group at the start of the experiment were equivalent. All pigs at the start of the trial (Day 0) were 28 day old male weaners.

IL-3 was provided in saline to inject 1 ml/pig.

Weights were measured at start, throughout the experiment and at the end of experiment. The trial continued for 133 days after the commencement ie. final weights were determined and animals slaughtered 133 days after the start of the weaning period. Weaning Days 0-42, Grower period Days 42-93, Finisher period Days 93-133. Treatments were administered during the weaner period only.

Blood and sera samples collected at start (before treatments) and end of the weaner period. Blood and sera were taken prior to injecting samples.

Materials and Methods

Recombinant porcine IL-3 was expressed in E. coli and purified using a polyHis tag system as described in Example 4. IL-3 was tested for biological activity in a bioassay prior to the start of the experiment.

Protocol Undertaken

| | |
|---|---|
| Day 0 | Weighed and grouped 28 day old |
| | weaners. |
| Day 1 | Bleed. Injected Groups. |
| Day 6 | Injected Groups. |
| Day 7 (Week 1) | Weighed |
| Day 9 | Injected Groups. |
| Day 13 | Injected Groups. |
| Day 14 (Week 2) | Weighed |
| Day 16 | Injected Groups. |
| Day 20 | Injected Groups. |
| Day 21 (Week 3) | Weighed |
| Day 23 | Injected Groups. |
| Day 27 | Injected Groups. |
| Day 28 (Week 4) | Weighed |
| Day 30 | Injected Groups. |
| Day 34 | Injected Groups. |
| Day 35 (Week 5) | Weighed |
| Day 37 | Injected Groups. |
| Day 41 | Injected Groups. |
| Day 42 (Week 6) | Weighed. Final bleed. Moved to |
| | grower pens. |
| (Days 42-93) | Grower stage. All pigs given |
| | standard feed and remained in |
| | previous groups. Weighed during |
| | (D73) and end of grower stage (D93). |
| (Days 93-133) | Finisher stage. Pigs moved into |
| | single pens and feed intake measured |
| | for FCR (food conversion ratio). |
| | All pigs given standard finisher |
| | feed. Weighed during (day of |
| | experiment(D) 114) and end of |
| | finisher stage (Slaughter D133). |
| | Measured final weight, P2 backfat. |

Notes:
+ medicated (antibiotics)feed and water.
− un-medicated (no antibiotics)feed and water.

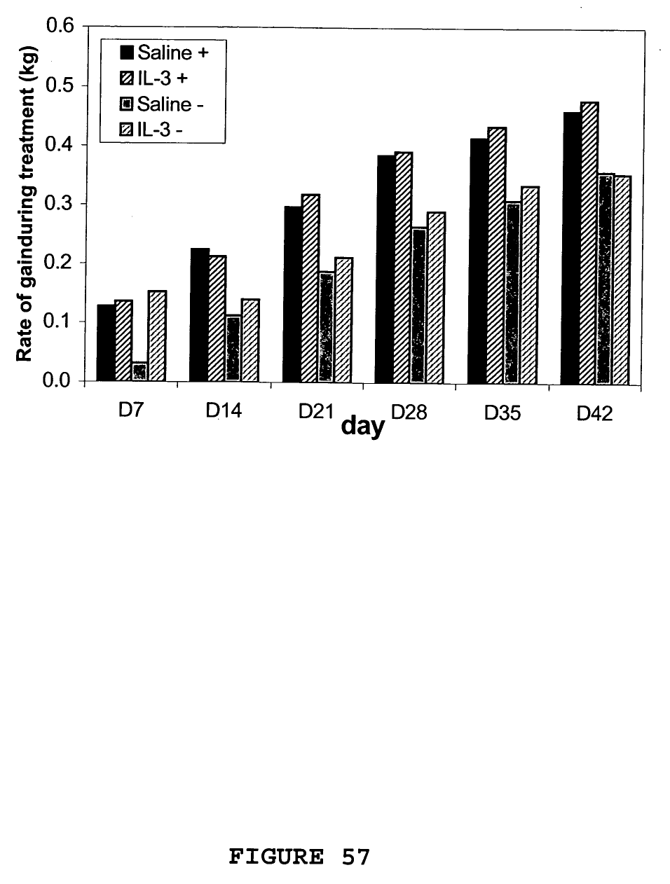

At the start of the trial, mean weights and variance were equalised between groups. FIG. 57 shows that IL-3 increased the rate of gain over the medicated and un-medicated saline controls, with the rate of gain consistently higher in the medicated groups compared to the un-medicated groups.

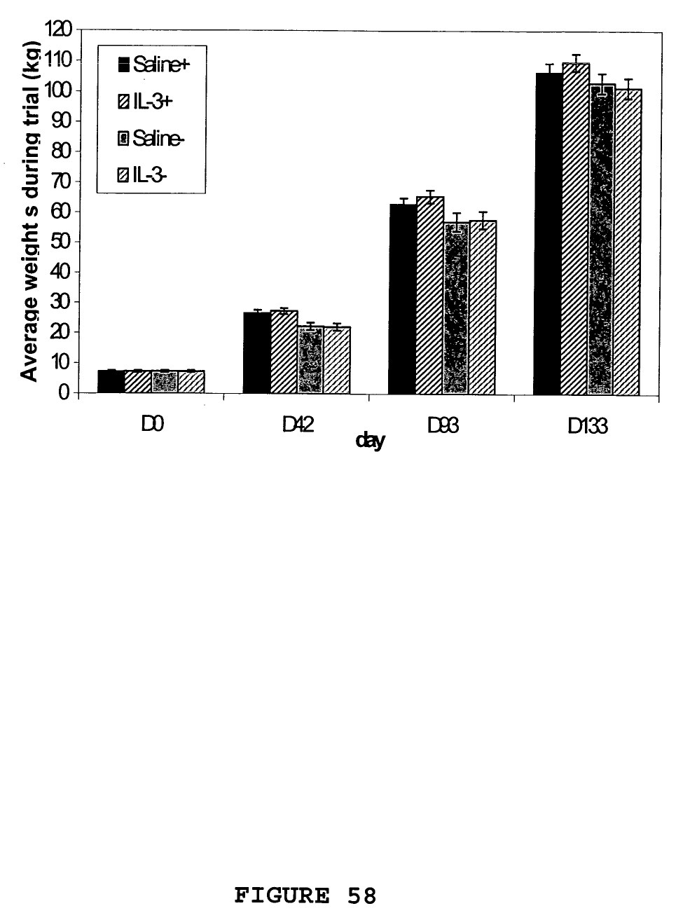

FIG. 58 shows that the IL-3 medicated group consistently showed higher average weights than all other groups. It also shows that the medicated pigs generally have higher average weights than un-medicated groups.

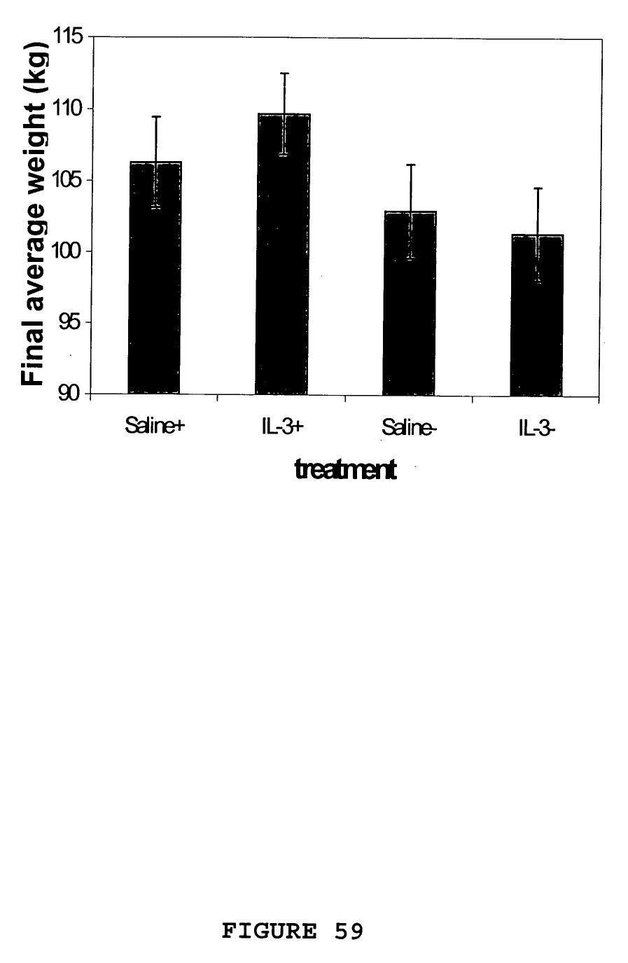

The average weight of pigs administered with IL-3 in the medicated group was over 3.5 kg higher than the saline medicated control average weight. The medicated groups had higher average weights than the un-medicated groups (approximately 3.5 kg difference between the medicated and un-medicated saline controls) (FIG. 59).

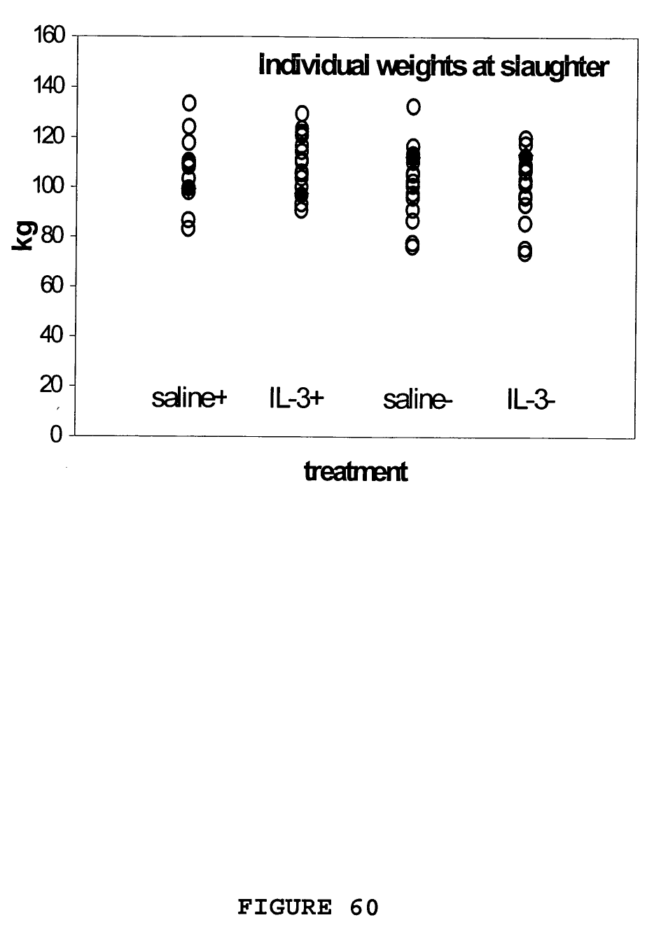

FIG. 60 shows that the IL-3 medicated pigs had a more consistent weight range than other groups which is an economic benefit to piggeries, especially which require less variation in final weights or carcass.

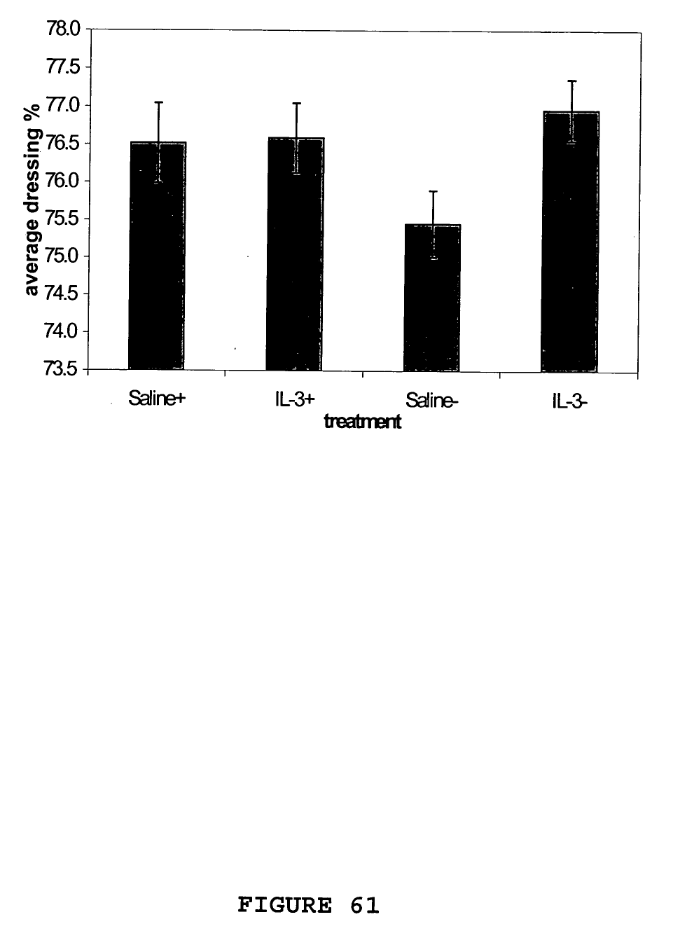

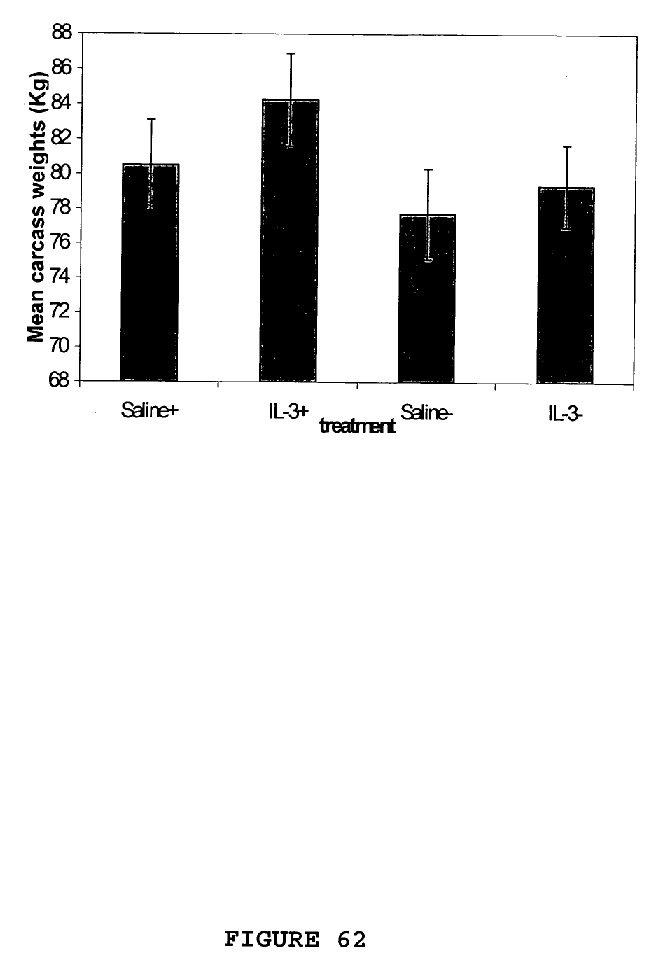

The IL-3 un-medicated pigs had a substantially higher % dressing than the respective un-medicated saline control and thereby a better carcass quality (FIG. 61). The average warm carcass weight at slaughter was also better for the IL-3 treated medicated and un-medicated pigs than the respective saline controls (approximately 4 kg/pig and 2 kg/pig) (FIG. 62). The medicated groups also had higher warm carcass weights than the un-medicated groups.

The FCR for the saline and IL-3 treated medicated pigs were similar eg. FCR Saline+2.50 and IL-3+2.52 (error bars overlap).

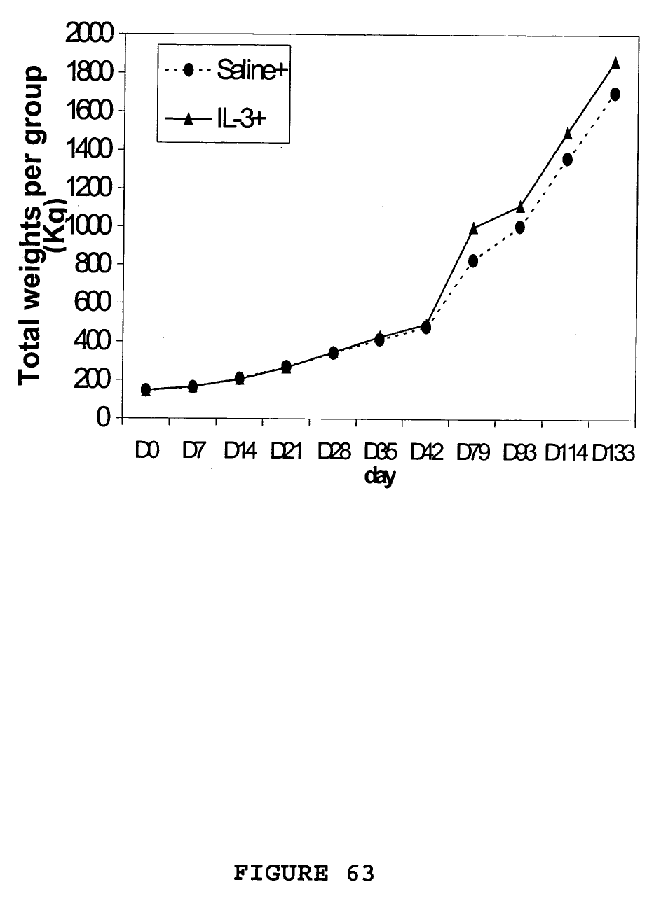

FIG. 63 shows that the IL-3 medicated group had a substantial increase in total weight (approximately 10% increase) of all pigs compared to the saline medicated group. Although the increase in total weights was not as apparent during the treatment period (weaner period, days 0-42), the duration of response continued post treatment period.

### EXAMPLE 9

### Examining the Effects of Porcine IL-3 on Blood Cell Populations

This trial examined the effect of administering recombinant porcine IL-3 protein on cell populations in the blood of pigs.

Protocol Undertaken

The experiment was conducted using medicated feed (Barastoc EziWean 150 then Bunge Grolean) ad libitum in an experimental environment (PC2 containment facilities).

Experimental Design

| Treatments | Administered |
|---|---|
| Group 1 | 4 pigs given 100 μg recombinant IL-3 |
| | daily for 5 days (days 0, 1, 2, 3, 4) |
| Group 2 | 4 pigs given 500 μg recombinant IL-3 on |
| | day 0 |
| Group 3 | 3 pigs given saline on day 0. |

Injections were administered intramuscularly in the hind leg. Pigs were 9 weeks of age at commencement of trial.

Blood samples were taken for haematology on days 0, 1, 2, 3, 4, 7, 9, 11, 15 and 17. Full haematology analysis was performed, using the Abbott Cell-Dyn 3700 and examination of selected smears were done for confirmation.

There was no significant changes or trends in total white blood cell counts, lymphocytes, monocytes, platelets, neutrophils or red blood cell counts (data not shown).

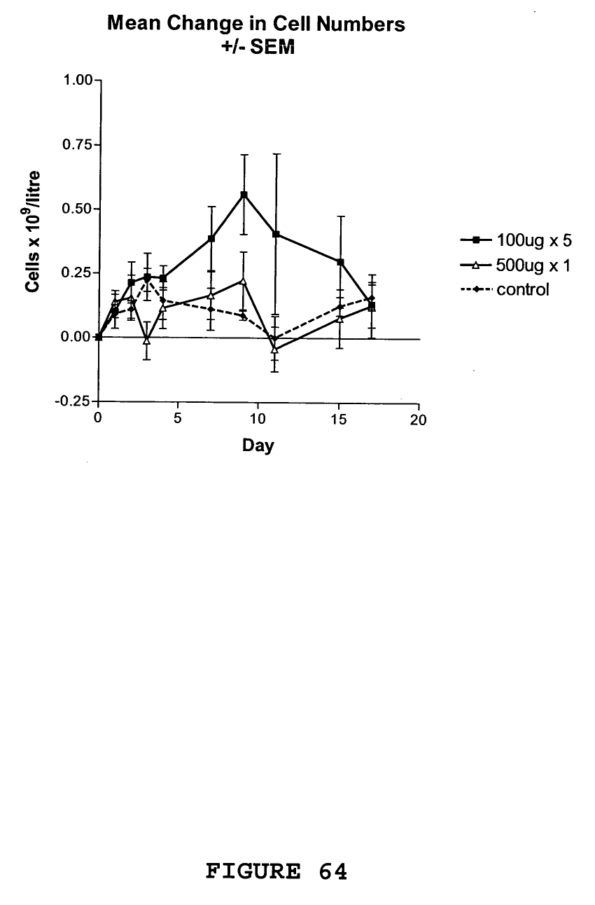

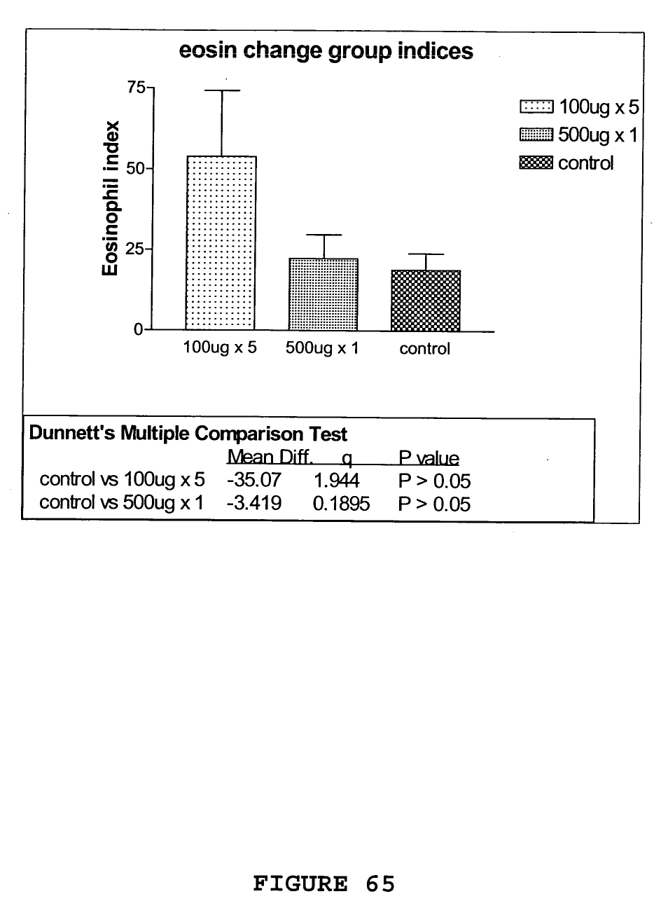

FIG. 64 shows that there was an increase in eosinophils in pigs given daily IL-3 and smaller increases in pigs given a single high dose of IL-3. Indices (calculated from the area under the curves) show the differences in group means; although there was a biological trend it was not statistically significant (FIG. 65).

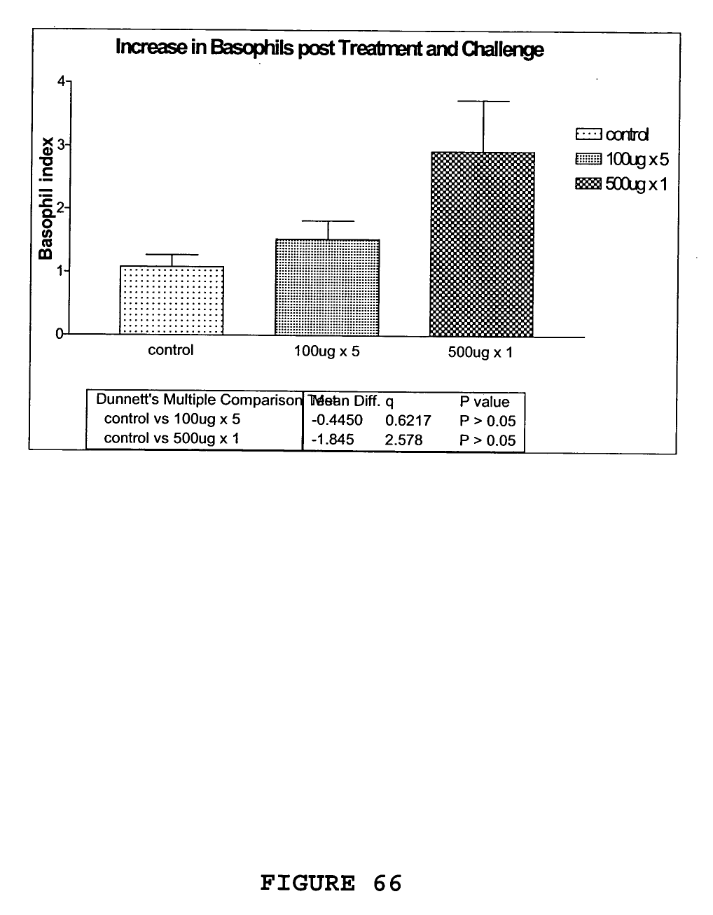

There appears to have been an increase in basophil numbers in the IL-3 treated groups, particularly the group that received a single high dose, although this may be biologically significant it was not statistically significant (FIG. 66).

### EXAMPLE 10

### Examining the Effects of Porcine IL-3 on Cell Populations Over a Longer Duration

This trial compared the effects of recombinant porcine IL-3 protein on eosinophil numbers in the blood of pigs over 8 weeks.

Protocol Undertaken

The experiment was conducted using medicated feed (Barastoc EziWean 150 then Bunge Grolean) ad libitum in an experimental environment (PC2 containment facilities).

Experimental design

| Treatments | Administered |
|---|---|
| Group 1 | 6 pigs given 100 μg recombinant IL-3 |
| | twice weekly for 2 weeks (days 0, 3, |
| | 7, 10) |
| Group 2 | 6 pigs given saline twice weekly for 2 |
| | weeks (days 0, 3, 7, 10) |

Injections were administered intramuscularly in the hind leg. Pigs were 5 weeks of age at commencement of trial. Blood samples were taken for haematology on days 0 (prior to injection), 1, 2, 3, 4, 7, 8, 10, 11, 15, 22, 29, and 57. Full haematology analysis was performed, using the Abbott Cell-Dyn 3700. Examination of selected smears was done for confirmation.

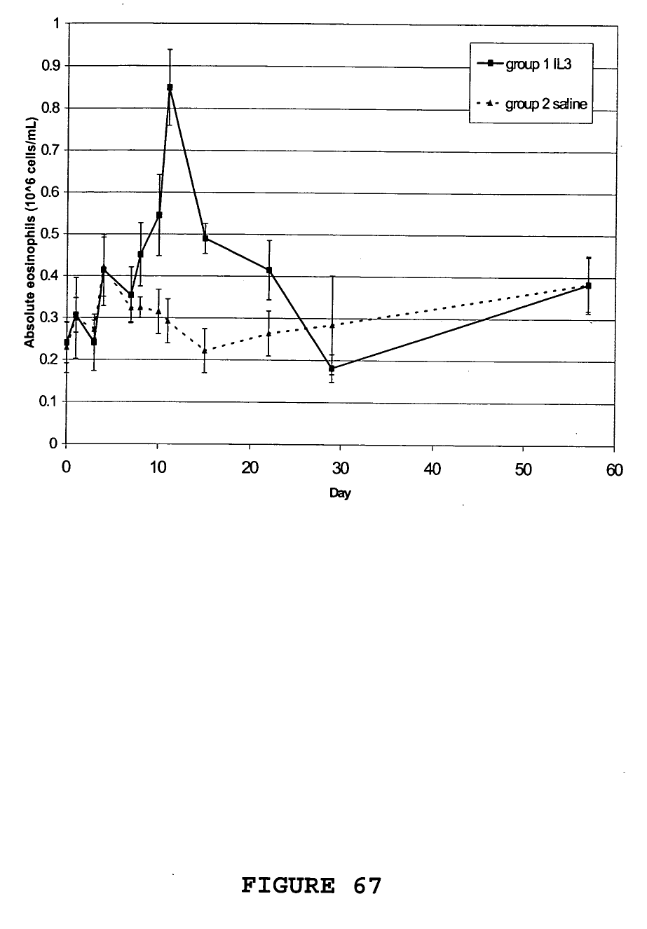

In a larger experiment with 6 pigs per group, there was a statistically significant increase in eosinophils in terms of absolute numbers of eosinophils in peripheral blood (FIG. 67) for an extended duration post cytokine administration.

No statistically significant trends were observed in this experiment for other cell types measured (total white blood cell counts, lymphocytes, monocytes, platelets, neutrophils or red blood cell counts, data not shown).

### EXAMPLE 11

### Synergistic Effects of IL-5 and IL-3 on Eosinophil Production

The aim of this example was to determine if the effects of IL-3 and IL-5 act synergistically to increase eosinophil levels and antibody production.

As IL-3 stimulates the proliferation of pre-active B cells prior to the effects of IL-5, it was believed that administration of both IL-3 and IL-5 would have a greater impact on eosinophil production than the administration of either cytokine alone. An experiment was undertaken using pigs raised under clean PC2 conditions with raised floors. There were 5 treatments with 6 pigs per treatment. Pigs were 5-6 weeks of age. The animals were injected intramuscularly on days 0, 3, 7 and 10 of the experiment with either recombinant cytokines or saline. The treatments include IL-5 and IL-3 alone and given either together at the same time at different sites or to the same animal but with IL-3 being administered one week before IL-5. The treatments were as follows:

Group 1—100 μg IL-3

Group 2—100 μg IL-5

Group 3—100 μg IL-3+100 μg IL-5 (separate sites)

Group 4—100 μg IL-3 (week 1); 100 μg IL-5 (week 2)

Group 5—Saline

The pigs were bled 4 times per week for the first 2 weeks and once per week for the following 2 weeks and haematology measured using a CellDyn machine. Sera were collected also each week and tested for antibody levels.

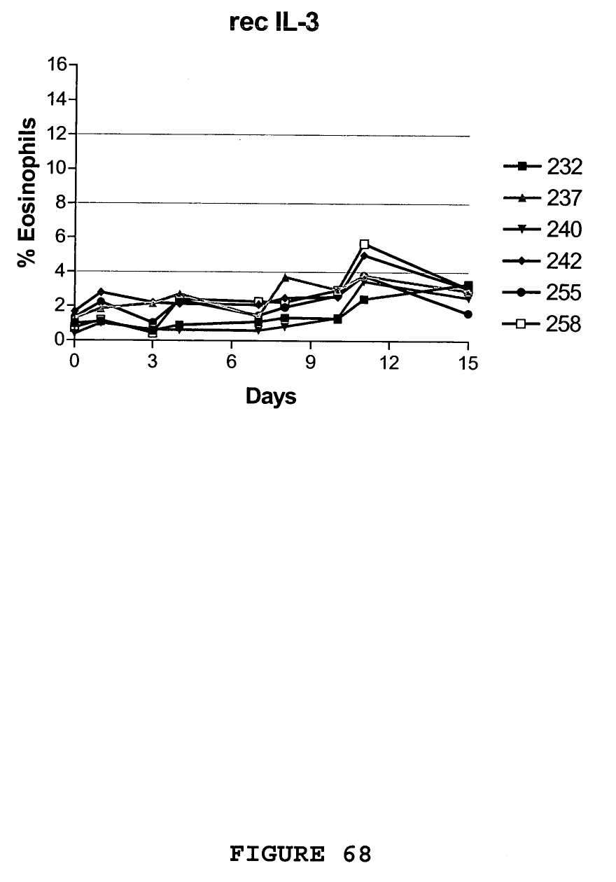

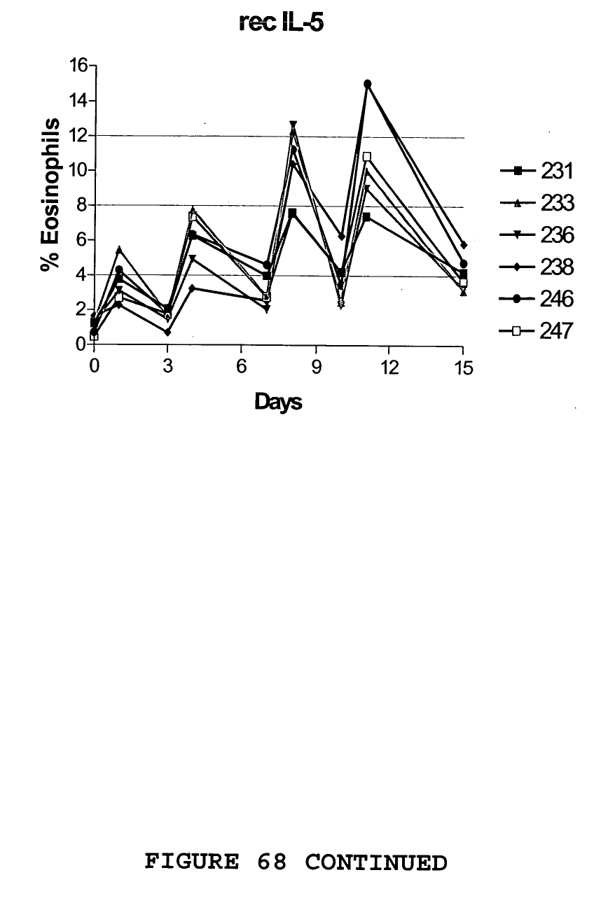

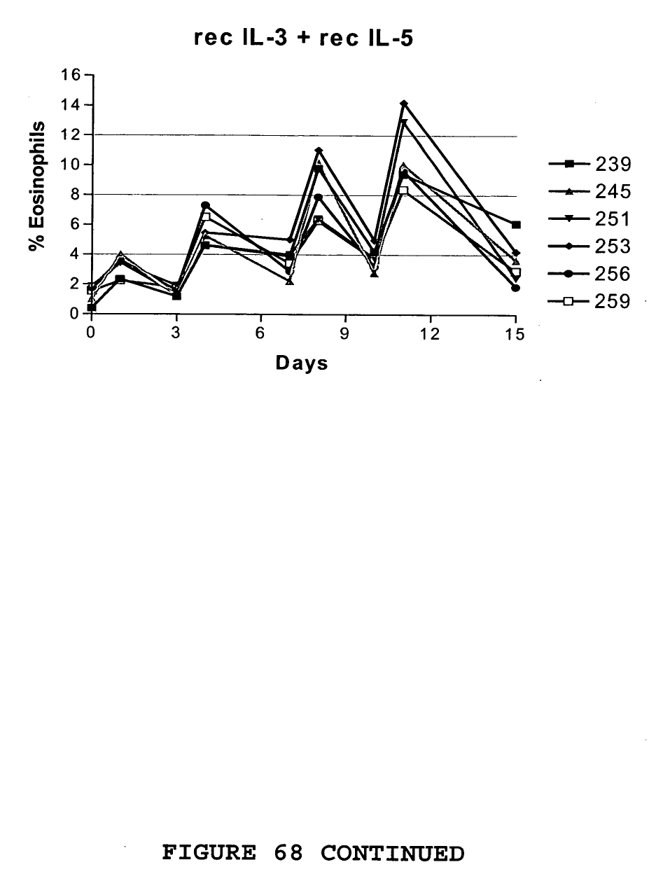

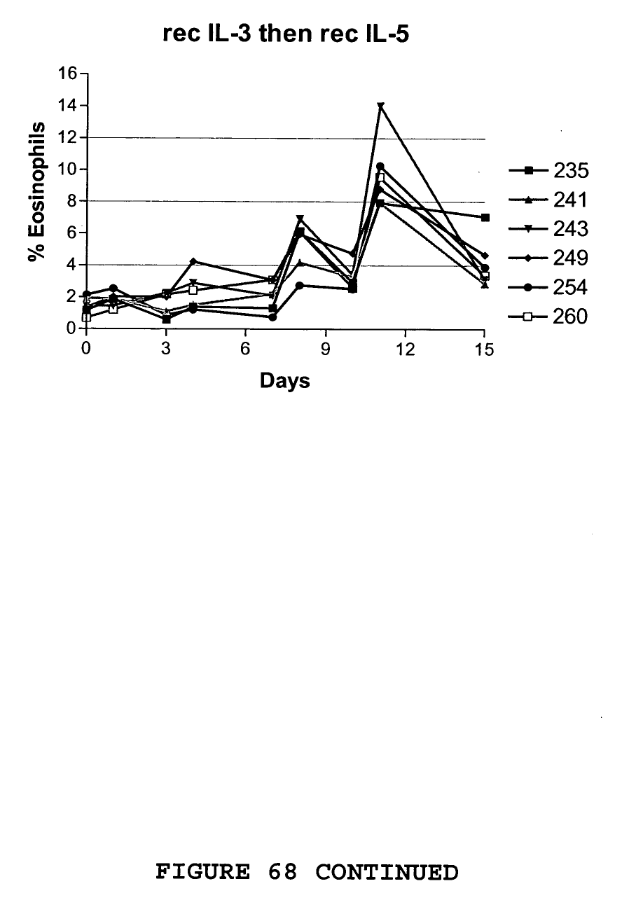

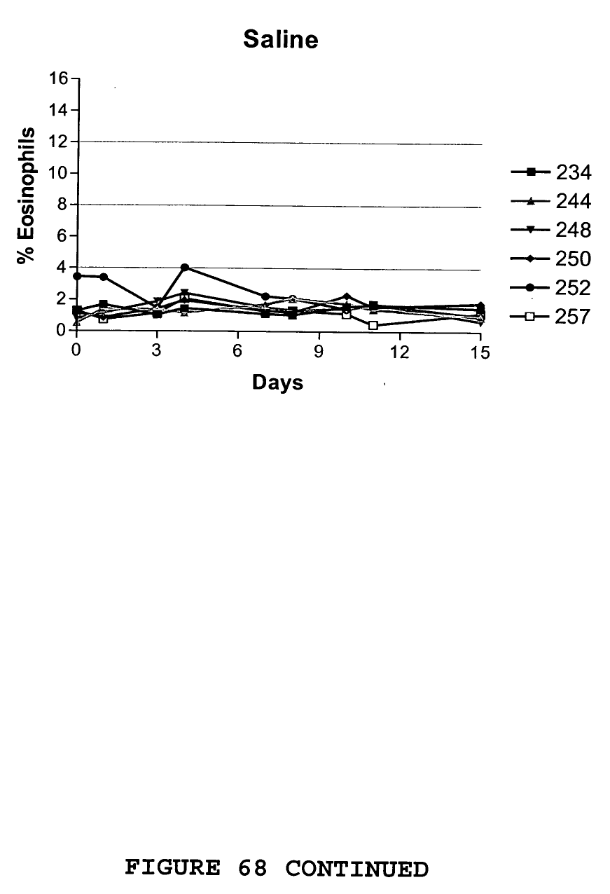

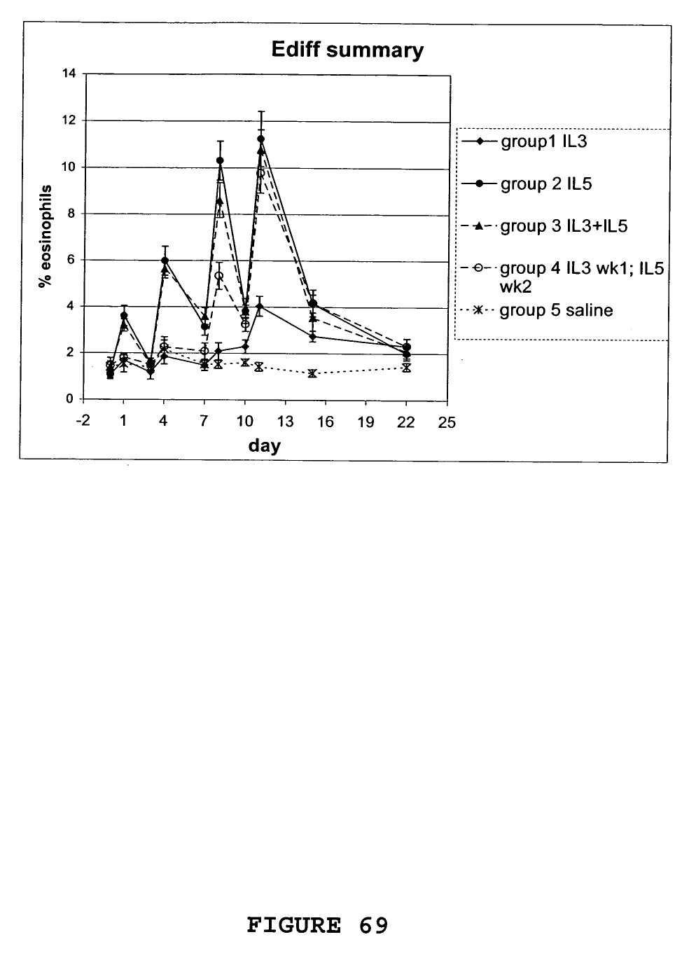

Analyses of total eosinophil numbers and eosinophils as a percent of white blood cells are shown in FIGS. 68 and 69. These figures show that IL-3 alone did not significantly increase the percentage of eosinophils, whereas IL-5 alone caused a significant increase in eosinophil levels. The eosinophil percentage of WBC increased with each repeated dose of IL-5 and after 4 injections was approximately 10 times the original value. There did not appear to be any synergy with IL-3 and IL-5 delivered together on eosinophil production (no higher than the IL-5 treated pigs). However, the treatment in the first week with 2 doses of IL-3 primed for a response to IL-5 delivered in the second week, so that 2 subsequent doses of IL-5 stimulated eosinophil levels that were equivalent to 4 doses of IL-5 (in non-IL-3 treated pigs). IL-3 is a hematopoietic cytokine that acts early on stem cells producing precursor cells including eosinophil precursors. These results indicated that IL-3 did increase eosinophil precursor cells enhancing the subsequent effects of IL-5.

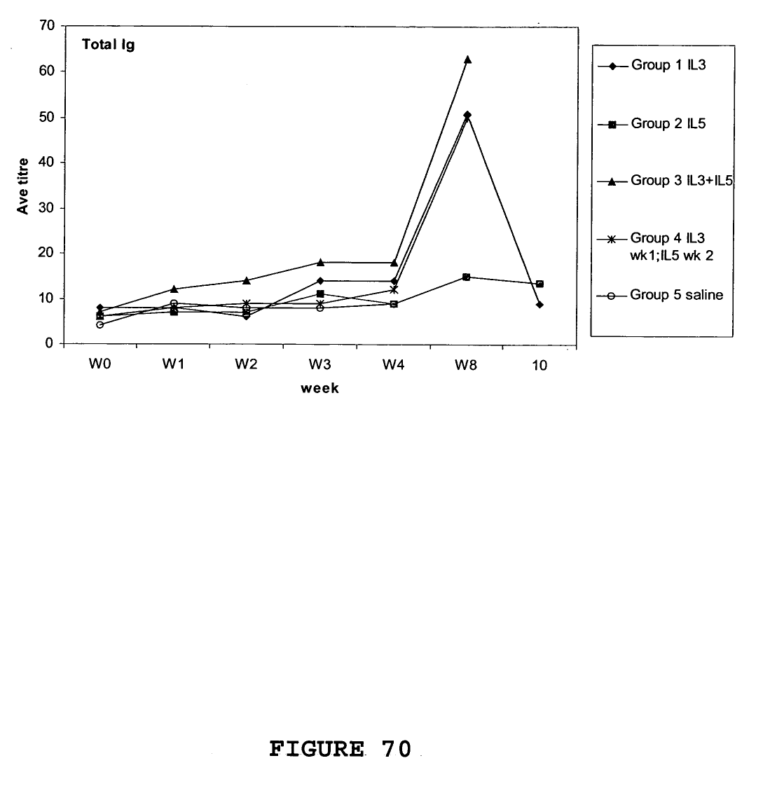

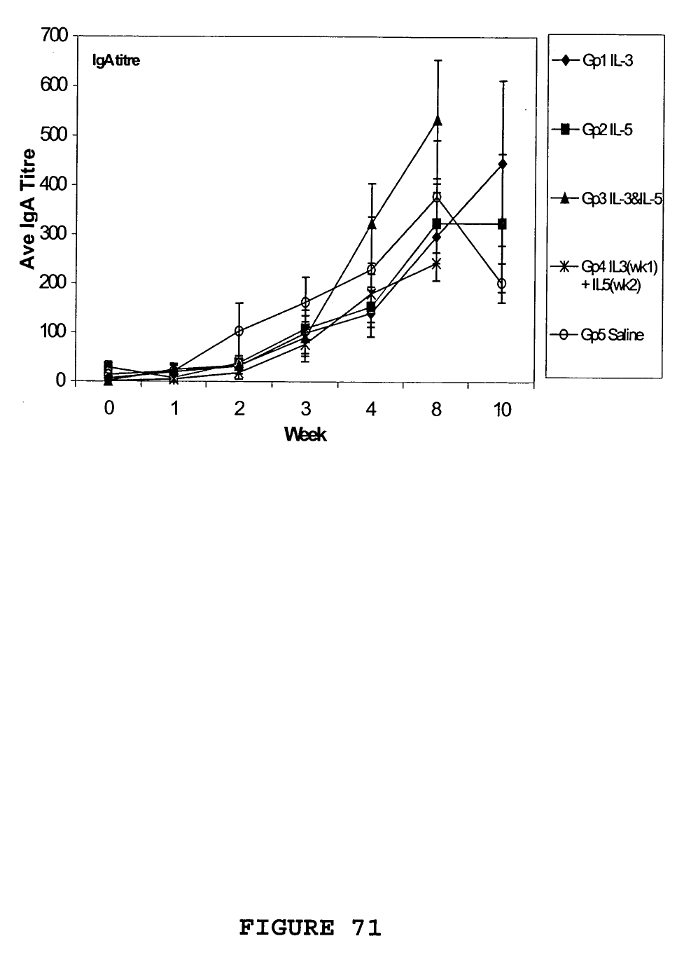

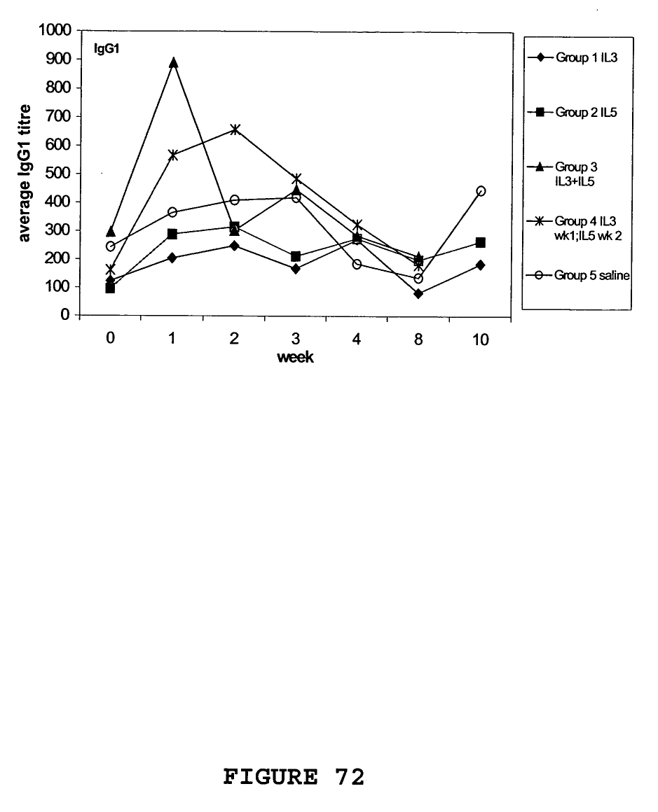

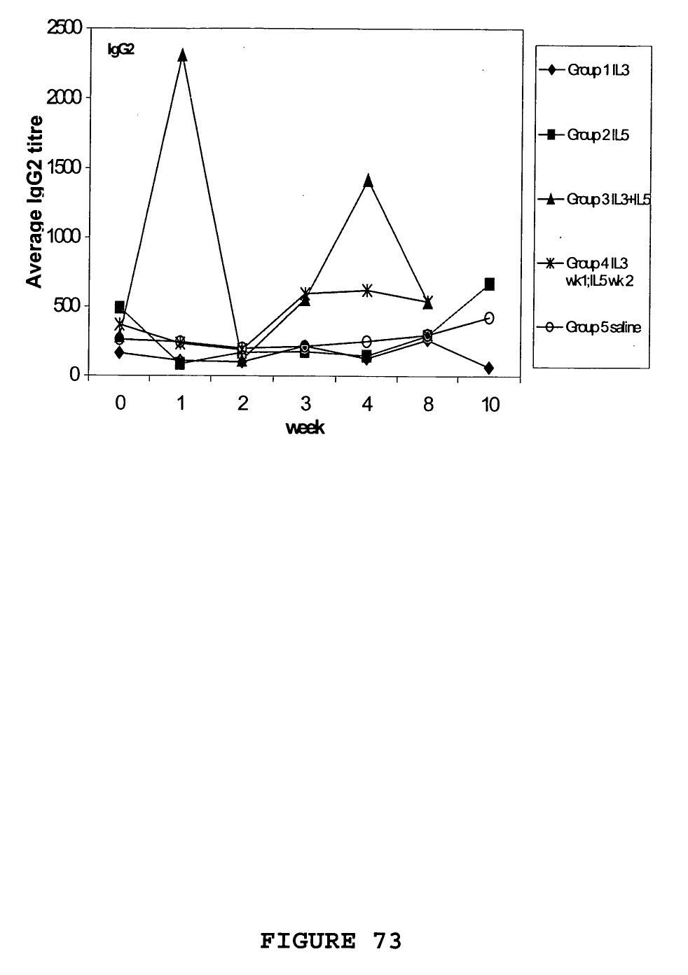

FIGS. 70-73 show the trends detected in average titres for each antibody isotype investigated. The error bars overlapped in each case and were not included. Generally, IL-3 +IL-5 had a greater stimulatory effect on B-cells as measured by antibody production than did IL-5 or IL-3 alone, suggesting an additive effect. This pattern was seen for total Ig (FIG. 70), IgA (FIG. 71) IgG1 (FIG. 72) and IgG2 (FIG. 73) isotypes, but not for IgM (data not shown).

### Conclusions

IL-5 dramatically increased circulating eosinophil cells, whereas IL-3 produced minor increases in comparison. IL-3 and IL-5 do not appear to act synergistically when administered together on eosinophil production; however, IL-3 appears to prime the response to IL-5 in terms of circulating eosinophil levels.

IL-3 and IL-5 appear to synergistically increase antibody production, although significant changes in antibody levels in sera were not detected with pigs kept in clean experimental conditions. It is noted from the literature that IL-5 increases IgA production only with bacterial endotoxin (eg. LPS). Presumably a commercial piggery environment would offer such a natural challenge of high endotoxin levels.

### EXAMPLE 12

### Delivery of Plasmids and Recombinant Cytokines to Improve Growth in Pigs Infected with Actinobacillus pleuropneumoniae

The following experiment was designed to determine whether IL-4 could improve the growth of immunologically challenged pigs compared to saline treated controls and positive controls treated with the non-steroidal anti-inflammatory drug (NSAID) Flunix. It was also devised to determine whether IL-4 could be delivered via plasmids.

Experiment Design

Male pigs, with a mean starting weight of 52 kg, were allocated to 5 treatment groups (Table 8). Pigs were housed in group pens, with each pen containing a replicate from each of the treatment groups. Pigs were provided with pelleted feed and water ad libitum.

Recombinant IL-4 and saline were administered as 2 ml doses, given subcutaneously behind the ear. Plasmids were administered in 1 ml doses, given intramuscularly in the hind-leg. Flunix was administered as a 2 ml dose according to the manufacturer's instructions, and delivered intramuscularly in the neck. The timetable of administration is outlined in Table 9 below.

TABLE 8
TREATMENTS AND DOSES APPLIED
IN CYTOKINE EXPERIMENT
(N = 4 PER GROUP).

| Treatment | Treatment Dose | |
|---|---|---|
| saline | 2 | ml |
| flunix | 2.2 | mg/kg |
| IL-4 | 100 | μg |
| plasmid control | 100 | μg |
| plasmid IL-4 | 100 | μg |

TABLE 9
PROTOCOL FOR EXPERIMENTAL PROCEDURES
TO ASSESS THE EFFICACY OF IL-4 AS A
PROPHYLACTIC TREATMENT ACTINOBACILLUS
PLEUROPNEUMONIAE INFECTION

| | Timing of | Timing of |
|---|---|---|
| | Administration | Administration |
| Event | (Room 1) | (Room 2) |
| plasmid | −10 days | −17 days |
| delivery | | |
| Recombinant | −2 days and day 0 | −2 days and day 0 |
| delivery | | |
| Challenge | Day 0 | Day 0 |
| clinical | 30 post-challenge | 24 post-challenge |
| visits | | |

Due to the intermittent availability of Actinobacillus pleuropneumoniae (App), the App challenge was performed separately on the 2 rooms, resulting in different time schedules for each room, as outlined in Table 9. Prior to each challenge, pigs were treated with recombinant cytokines, flunix or plasmids as described above; the timing of treatments with respect to challenge is described in Table 9. Pigs were anaesthetised and infected intratracheally with 7.5×10⁵ pfu on day 0.

Blood was sampled from pigs by venipuncture at 0 h, 24 h and 14 days post-challenge. Blood was assayed for immunological parameters as previously described.

Briefly, assays were performed on blood samples using standard techniques, including: white blood cell counts performed using an automated cell counter; differential cell counts performed manually on stained blood smears; lymphocyte subset enumeration via flow cytometry; neutrophil function determined by flow cytometry; lymphocyte proliferation determined using a thymidine incorporation assay in response to mitogens; total IgG and IgA levels were identified using indirect sandwich ELISA; levels of mRNA for pro-inflammatory cytokines was detected by RT-PCR. TNF levels were additionally measured in serum by bioassay using L929 target cells. Pigs were weighed weekly from delivery of plasmids and for 2 weeks after challenge.

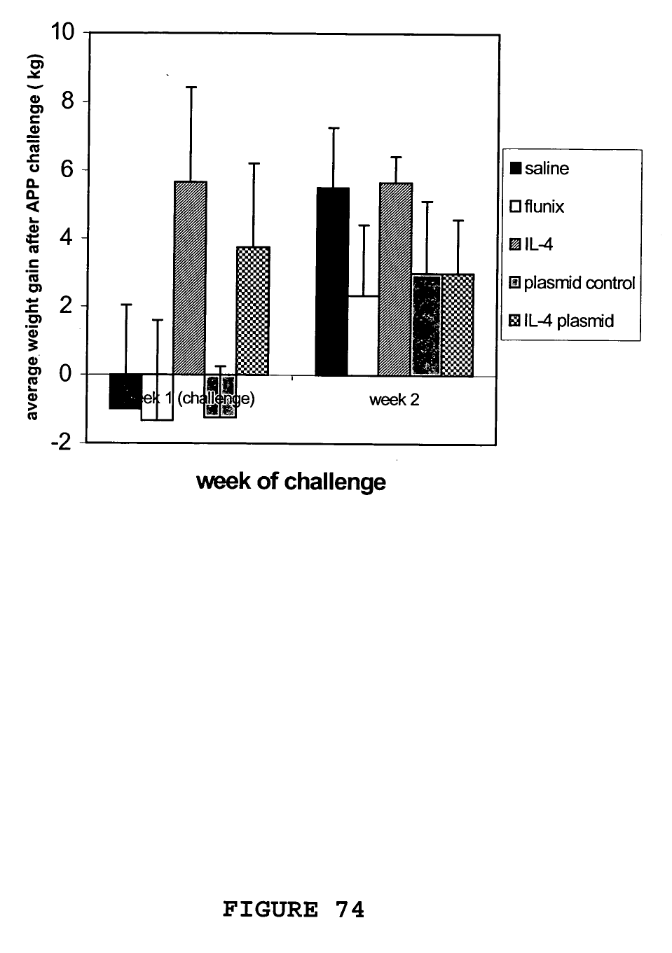

During the week of challenge, IL-4 improved the growth of pigs (FIG. 74) compared to saline-treated controls. Pigs treated with saline, flunix, or control plasmid showed weight loss, while pigs treated with IL-4 or plasmid IL-4 showed positive growth during the week of challenge. In the week following challenge, all groups of pigs gained weight. Pigs treated with saline recovered significantly, while pigs treated with IL-4 continued to gain weight. Pigs treated with plasmids or flunix had the poorest growth of all groups in the second week of challenge.

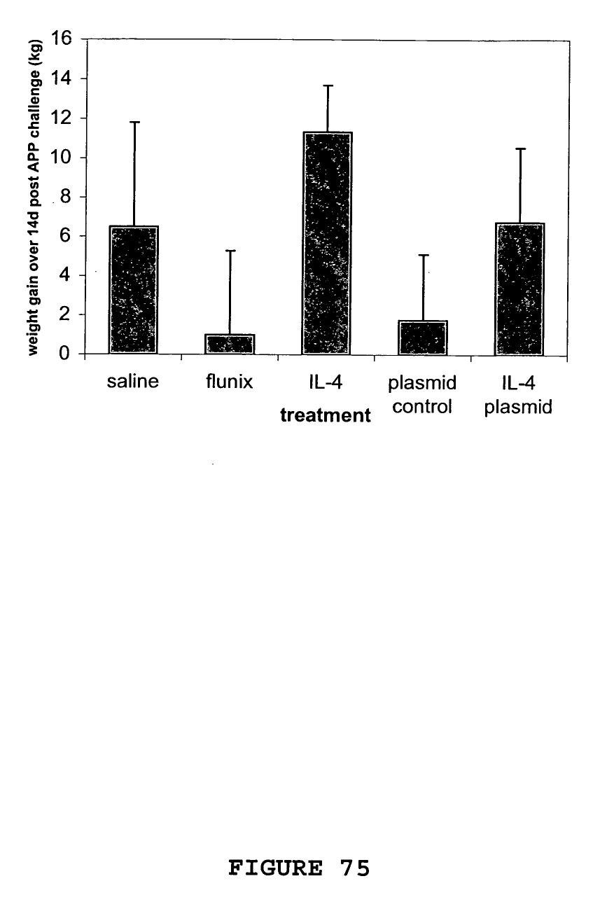

Weight gain over the 2 week period following challenge with App (FIG. 75) showed that recombinant IL-4 treatment increased weight gain compared to saline-treated controls, although this result was not statistically significant. There was a difference in weight at slaughter. Flunix was the poorest performing treatment in terms of growth, compared to saline-treated controls. Pigs treated with IL-4 plasmid had considerably enhanced growth over the entire 2-week challenge period compared to their plasmid treated controls (FIG. 75) but equivalent to the growth of saline treated controls.

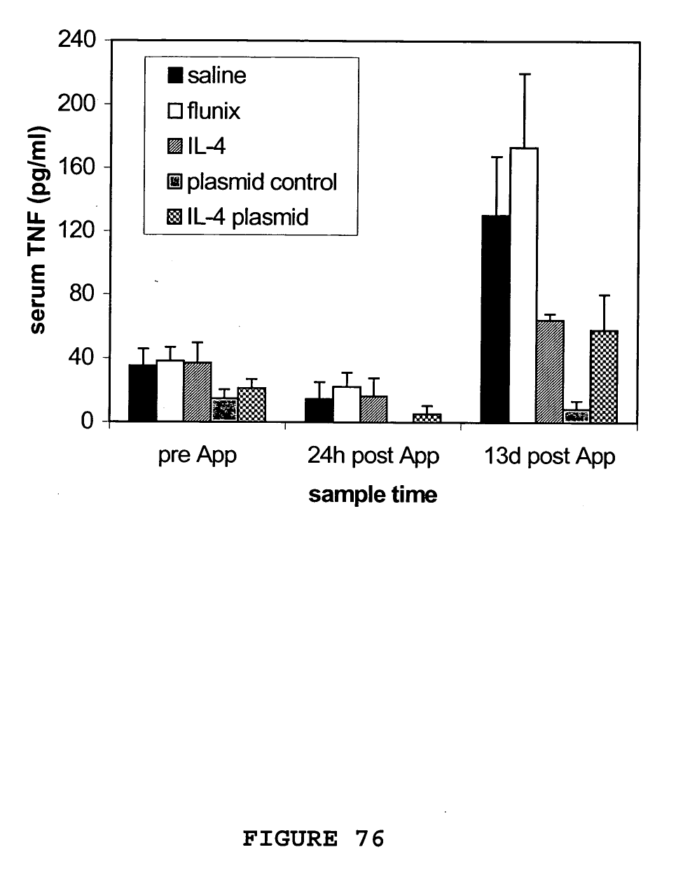

Pro-inflammatory cytokines, TNFα and IL-6 were elevated in several groups after challenge with App, compared to pre-challenge levels. Interestingly, the NSAID flunix, failed to inhibit the production of TNFα (FIG. 76), which may help to explain the poor growth seen in this group. IL-4, plasmid control and IL-4 plasmid had reduced levels of TNF production than did saline-treated and flunix-treated controls at day 13 after challenge.

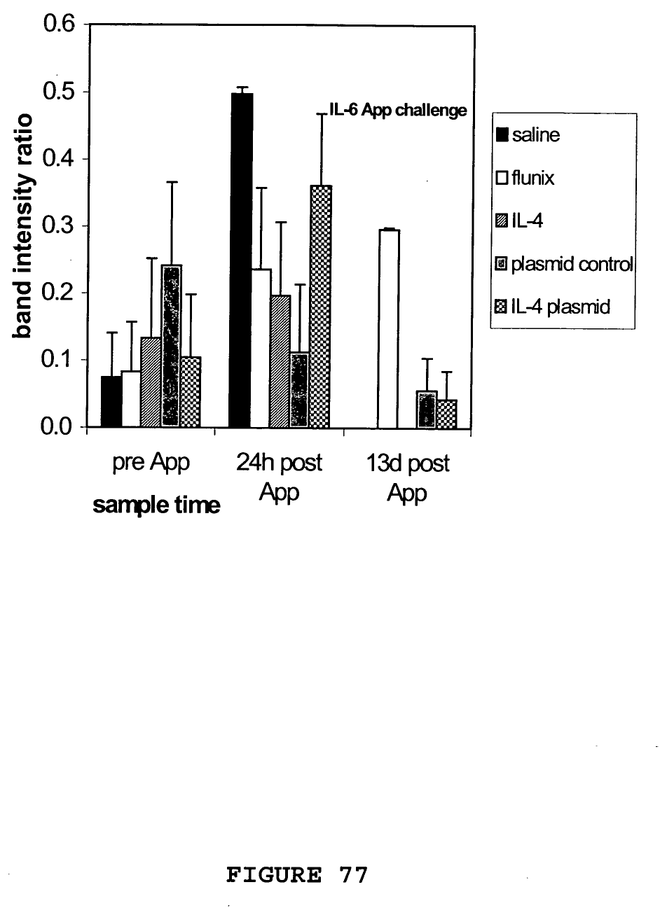

All treatments reduced the production of IL-6 24 h after challenge compared with saline treated controls (FIG. 77). Unfortunately, IL-6 data was not retrievable for the saline treatment at 13 days after App challenge due to sampling error. After 13 days of challenge, pigs treated with IL-4 as either plasmid or recombinant, had reduced levels of the pro-inflammatory cytokine, IL-6, compared to pigs treated with flunix. Plasmid IL-4 did not reduce the production of IL-6 compared to the plasmid control. Recombinant IL-4 reduced IL-6 production to undetectable levels at day 13 after challenge with App.

While the anti-inflammatory cytokine treatments did cause reductions in the levels of pro-inflammatory cytokines in the circulation, and in some cases improved growth, the relationship between pro-inflammatory cytokines and impaired growth is still unclear. However, importantly, groups of pigs with reduced levels of pro-inflammatory cytokines were typically the groups that also had the least inhibition of growth in the first week after challenge.

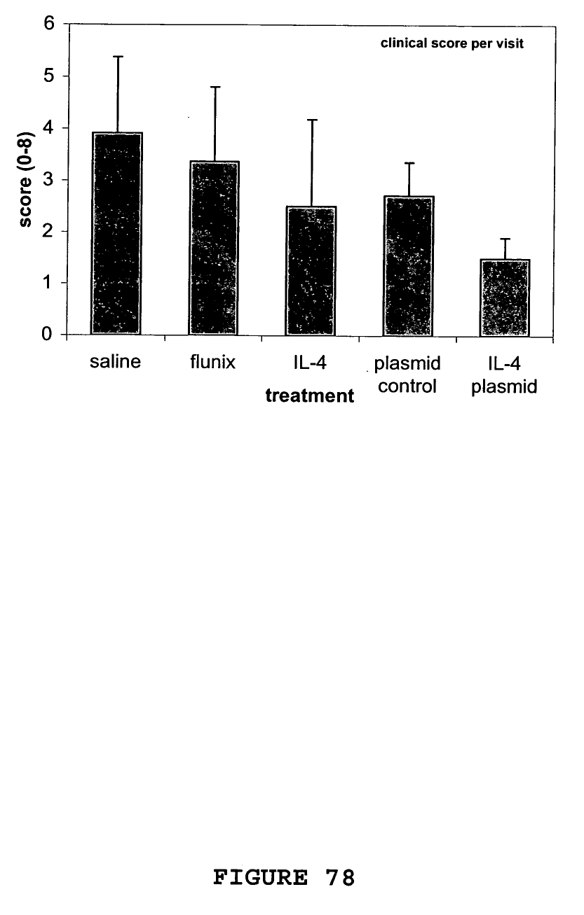

In addition to improving the growth of pigs, we found that cytokine treatment could improve the health of pigs exposed to App challenge. The data in FIG. 78 shows mean clinical scores per visit over 30 visits conducted during the first week of challenge. The severity of symptoms displayed by each pig, such as lethargy, coughing and breathing parameters was scored from 0-8, and pigs which died or were euthanased were arbitrarily given a score of 8 at each subsequent visit. Pigs treated with recombinant IL-4 had slightly reduced clinical signs of disease compared to saline-treated controls (FIG. 78). IL-4 delivered as a plasmid also resulted in reduced clinical symptoms compared to saline and plasmid control pigs. IL-4 delivered as a recombinant caused a reduction of 36% in the presence of clinical symptoms compared to pigs treated with saline, while IL-4 delivered in plasmid form produced a reduction of 62% compared to saline-treated controls (45% reduction compared to plasmid-treated controls). IL-4 delivered as plasmid or recombinant was more effective than flunix in reducing the clinical symptoms of App infection.

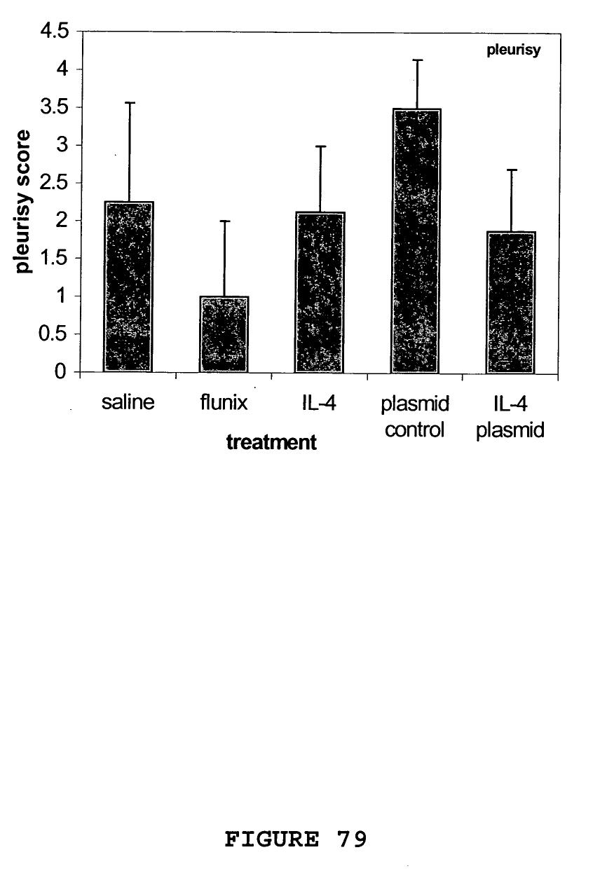

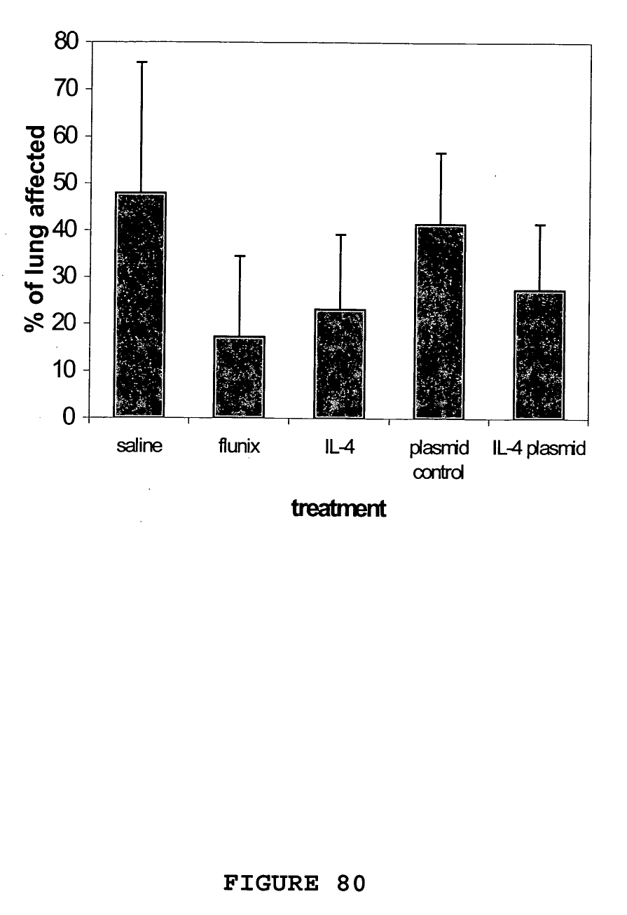

At the conclusion of the trial, pigs were euthanased and the lungs removed for post-mortem examination. Lungs were scored for pleurisy from 0-5 (FIG. 79) and the degree of pleuropneumonia was determined by weighing affected lung and expressed as a percentage of total lung weight (FIG. 80). Pigs treated with flunix had less pleurisy than the saline controls. Pigs treated with IL-4 had the same level of pleurisy as saline-treated controls. Although pigs treated with IL-4 delivered as plasmid had less pleurisy than their plasmid-treated controls, their level of pleurisy was not less than that of saline-treated controls (FIG. 79). The percentage of lung affected by App lesions was greatly reduced in pigs treated with flunix, recombinant IL-4 or plasmid IL-4 compared with saline-treated controls (FIG. 80).

### Conclusions

Recombinant IL-4 was able to greatly increase the growth of pigs compared to saline treated controls during the first week of App challenge. Pigs treated with IL-4 were subsequently 4.8 kg heavier at the termination of the experiment, after 2 weeks of challenge than their saline treated peers, which represents an improvement in growth of 73%. Pigs treated with flunix had the lowest growth over the 2 week challenge period.

Plasmid IL-4 was able to improve the growth of pigs compared to saline treated controls and plasmid treated controls during the first week of App challenge. At the conclusion of the 2 week challenge trial, IL-4 plasmid pigs were heavier than their plasmid-treated counterparts, but equal in weight to saline-treated pigs.

Recombinant IL-4, plasmid control and plasmid IL-4 were able to reduce the production of the pro-inflammatory cytokines TNFα and IL-6 which are associated with poor growth performance. Flunix was able to reduce the production of IL-6 only.

IL-4 reduced the severity of clinical symptoms of disease during the challenge, as did IL-4 delivered as plasmid.

Flunix was able to reduce the level of pleurisy seen at post-mortem. Flunix, recombinant IL-4 and plasmid IL-4 all reduced the percentage of lung affected by App lesions, compared to saline-treated and plasmid-treated controls.

### EXAMPLE 13

### Delivery of Recombinant IL-4 as a Prophylactic

The aim of this example was to determine whether IL-4 could improve the health of pigs infected with an enteric inflammatory pathogen causing swine dysentery, Brachyspira (Serpulina) hyodysenteriae. A further aim was to determine whether IL-4 could improve the growth rate of pigs under conditions of challenge with swine dysentery.

Experiment Design

Male pigs with a mean starting weight of 6.5 kg, were allocated to treatment groups consisting of eight pigs (Table 10). Pigs were housed in group pens, with each pen containing a replicate from each of the treatment groups. One group of 8 pigs was housed in a separate room and left uninfected to act as untreated controls. Pigs were provided with pelleted feed and water ad libitum.

Prior to swine dysentery challenge, pigs were treated with recombinant IL-4 or saline, as described in Table 10. Cytokines and the antibiotic, Lincocin, were delivered by intramuscular injection at intervals outlined in Table 11. Pigs were infected with Brachyspira hyodysenteriae at day 0, day 1 and day 2, given as an oral bolus of 120ml of spirochaete culture in log phase of growth, containing approximately 108 cells.

Faecal swabs and blood samples were taken from each pig at intervals described in Table 11. Faecal swabs were cultured for the presence of spirochaetes. Blood samples were assayed for immunological parameters as described in example 1 above. Pigs were weighed at weekly intervals throughout the experiment, which was terminated by euthanasia on days 19 and 20 after the initial challenge. At post mortem swabs from areas of the hindgut were cultured for the presence of spirochaetes, and the gross pathological condition of the gastro-intestinal tissue noted.

TABLE 10
SUMMARY OF TREATMENT GROUPS FOR
CHALLENGE TRIAL TO ASSESS THE EFFICACY OF
IL-4 AS A PROPHYLACTIC TREATMENT FOR SWINE
DYSENTERY INFECTION (N = 8 PER GROUP)

| Treatment | Treatment Dose |
|---|---|
| saline | 1 ml |
| IL-4 | 1 ml @ 200 ug/ml |
| Lincocin | 2 ml (as per manufacturers |
| | instructions) |
| untreated | No treatment, no challenge |

TABLE 11
PROTOCOL FOR EXPERIMENTAL PROCEDURES TO ASSESS
THE EFFICACY OF IL-4 AS A PROPHYLACTIC TREATMENT
FOR SWINE DYSENTERY INFECTION

| | | | | | Inject 1 ml | Inject | |
|---|---|---|---|---|---|---|---|
| | | Faecal | | | IL-4, or | 2 ml | |
| Day | Weigh | Swabs | Infect | Bleed | Saline | Lincocin | Kill |
| −7 | X | | | | | | |
| −6 | | | | | | | |
| −5 | | | | | | | |
| −4 | | | | | | | |
| −3 | | | | | | | |
| −2 | | | | | X | | |
| −1 | | | | | X | | |
| 0 | X | X | X | X | X | | |
| 1 | | | X | | X | X | |
| 2 | | | X | | X | X | |
| 3 | | | | | | X | |
| 4 | | | | | | | |
| 5 | | X | | X | X | X | |
| 6 | | | | | | X | |
| 7 | X | X | | | X | X | |
| 8 | | | | | | X | |
| 9 | | X | | | X | X | |
| 10 | | | | | | | |
| 11 | | | | | | | |
| 12 | | X | | X | X | X | |
| 13 | | | | | | X | |
| 14 | X | X | | | X | X | |
| 15 | | | | | | X | |
| 16 | | X | | | X | X | |
| 17 | | | | | | | |
| 18 | | | | | X | X | |
| 19 | X | | | | | | X |
| 20 | X | | | | | | x |

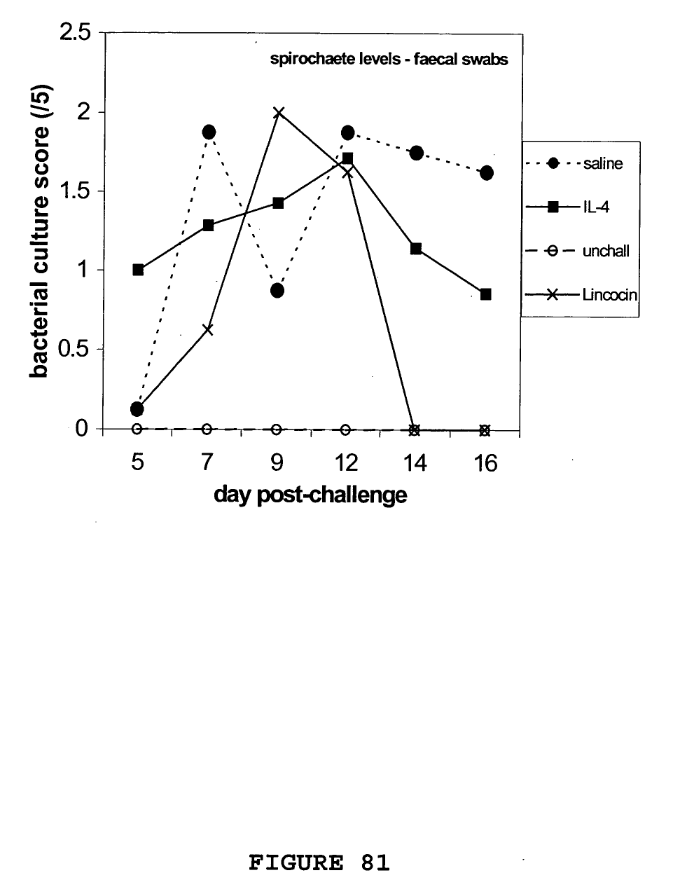

All groups of pigs infected with swine dysentery were shedding spirochaetes in faeces from day 5 after challenge as detected by faecal culture (FIG. 81). Pigs treated with IL-4 showed decreasing levels of spirochaete shedding by day 14 after challenge, compared to the saline treated control group, suggesting that IL-4 treated animals were resolving their infection more quickly than saline treated controls. Pigs treated with the antibiotic Lincocin showed no signs of faecal spirochaete shedding by day 14. Unchallenged pigs did not shed any spirochaetes for the duration of the trial (FIG. 81).

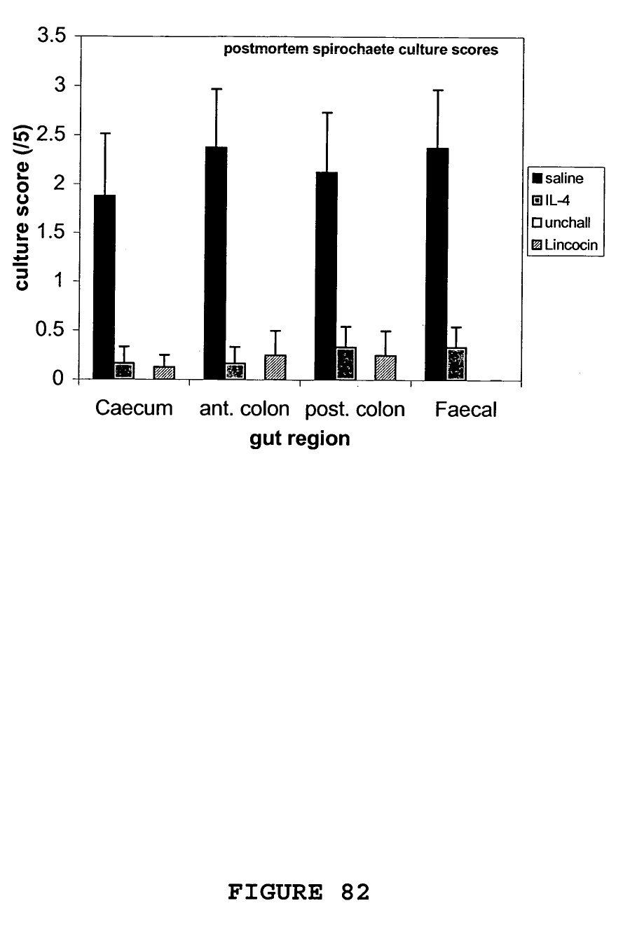

Spirochaete cultures taken from the hindgut at post mortem show that treatment of pigs with IL-4 significantly reduced the number of spirochaetes residing in the gut compared to saline controls (P<0.05; FIG. 82). IL-4 was able to reduce spirochaete culture scores in the caecum, anterior colon, posterior colon and faeces compared to saline treated controls. Importantly, IL-4 performed as well as the Lincocin antibiotic treatment in reducing the number of spirochaetes in the caecum and colon at post mortem. As expected, pigs that were not challenged with swine dysentery did not have spirochaetes in their hindgut or faeces at post mortem.

Compared to saline treated pigs, IL-4 treatment resulted in a 91% reduction in the number of spirochaetes in the caecum, 93% reduction in the anterior colon, 84% in the posterior colon and 86% reduction in faecal spirochaetes.

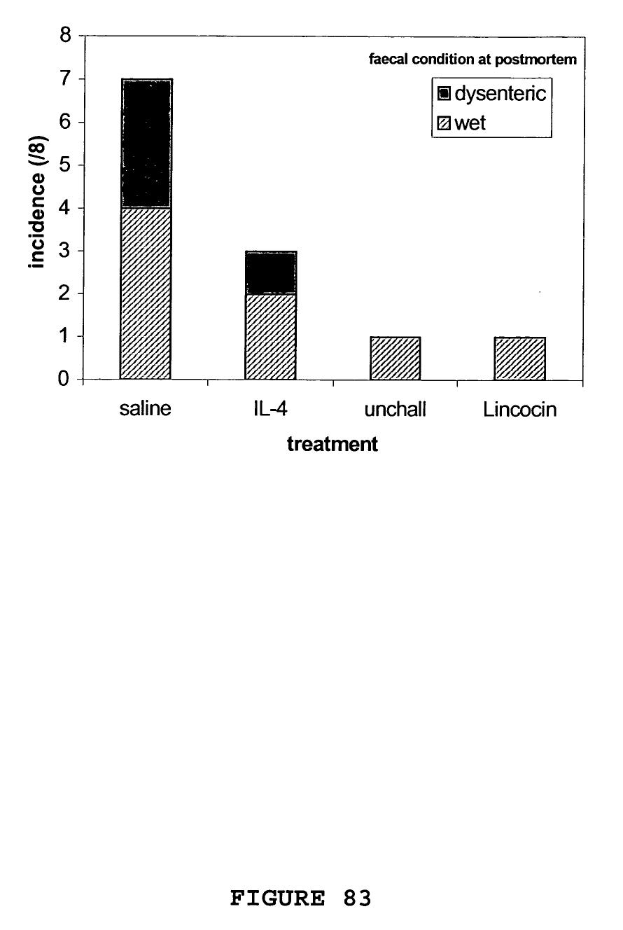

In addition to a reduction in the number of spirochaetes in the gut, treatment with IL-4 also reduced the clinical signs associated with infection indicated by faecal condition. FIG. 83 shows that IL-4 treated pigs showed fewer signs of dysentery-affected faeces (wet and mucoid with blood) or wet faeces (abnormally wet faeces unable to hold form) compared to saline treated controls. Of the 8 saline treated pigs, 7 showed clinical manifestation of swine dysentery determined by faecal condition, 3 of which were dysenteric mucoid and bloody in nature. Treatment with IL-4 reduced the incidence of clinical signs in faeces to 3 from 8 pigs infected, with only 1 pig showing signs of bloody and mucoid faeces associated with severe infection with swine dysentery (FIG. 83). Pigs treated with the Lincomycin antibiotic had negligible clinical signs of infection with only one of the 8 pigs in this group showing wet faeces, which was a comparable result with the uninfected control pigs.

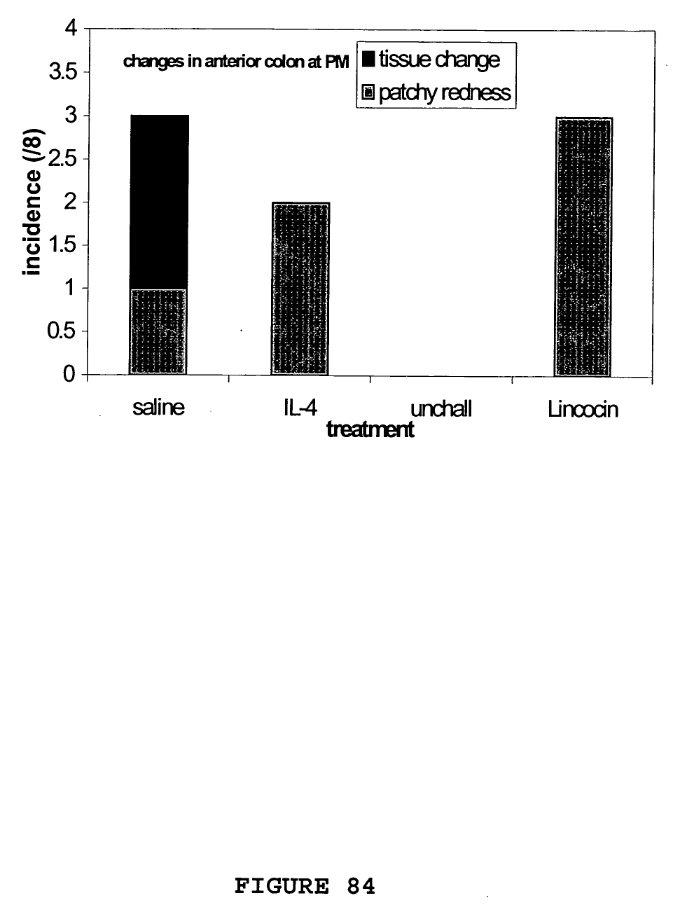

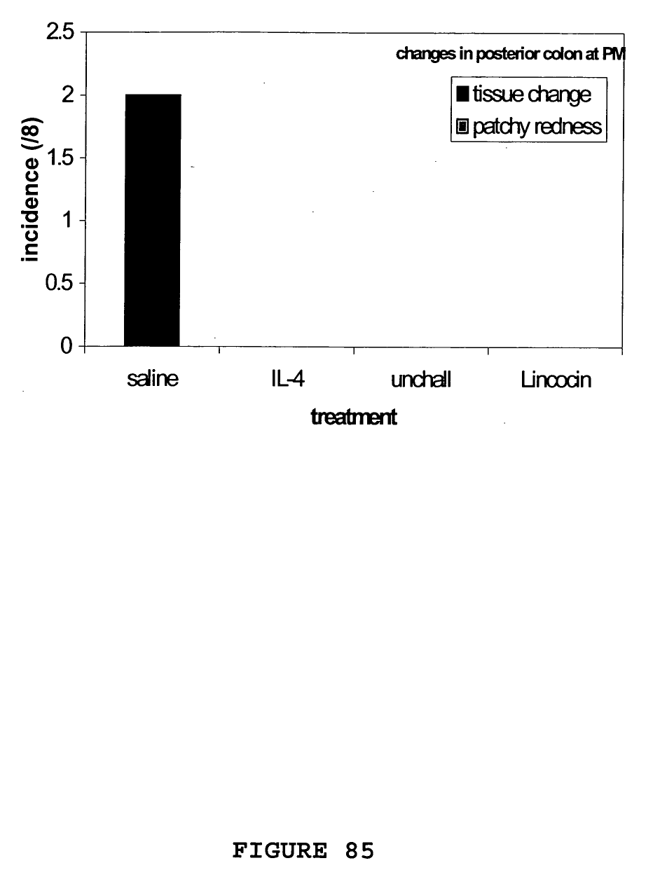

As expected with differences in the number of spirochaetes and the presence of clinical signs noted between treatment groups, there were also differences in the degree of infection-associated pathology seen in the gut at post mortem (FIGS. 84 & 85). Treatment with IL-4 reduced the signs of pathology associated with dysentery, and the severity of the pathology compared to saline in the anterior colon, and completely prevented the development of pathology in the posterior colon. Treatment with Lincocin reduced the incidence of pathological symptoms in anterior and posterior colon while it reduced the severity of pathological changes in the caecum (data not shown).

Such results confirmed that IL-4 and Lincocin were both able to reduce the deleterious effect of swine dysentery infection on the health of pigs. IL-4 was known to have anti-inflammatory effect on the immune system, thus, a reduction in inflammatory pathological changes in the gut associated with dysentery may be attributable to both the anti-inflammatory properties of this cytokine and a reduced spirochaete load (as seen in FIG. 82).

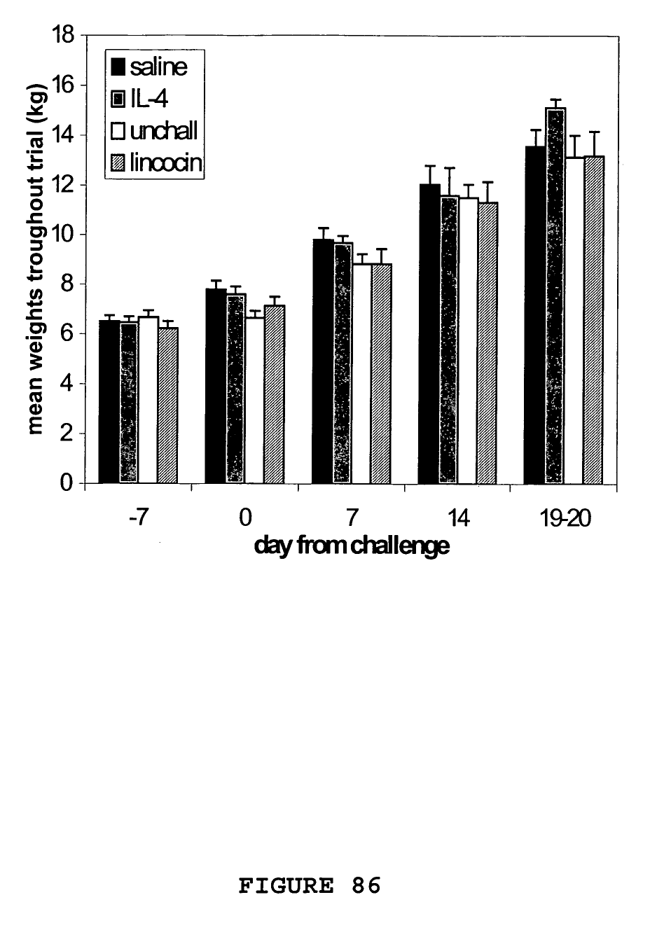

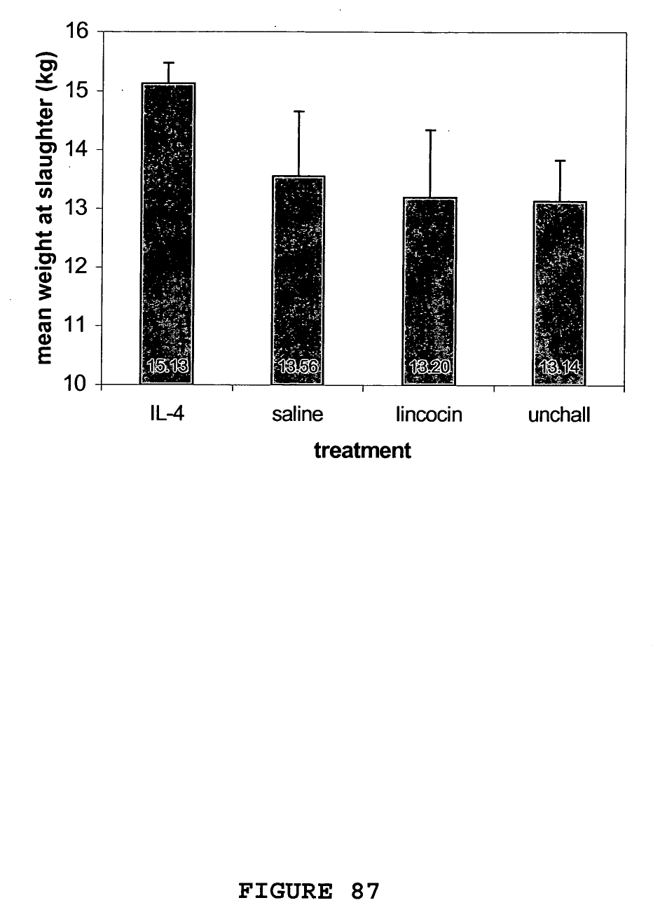

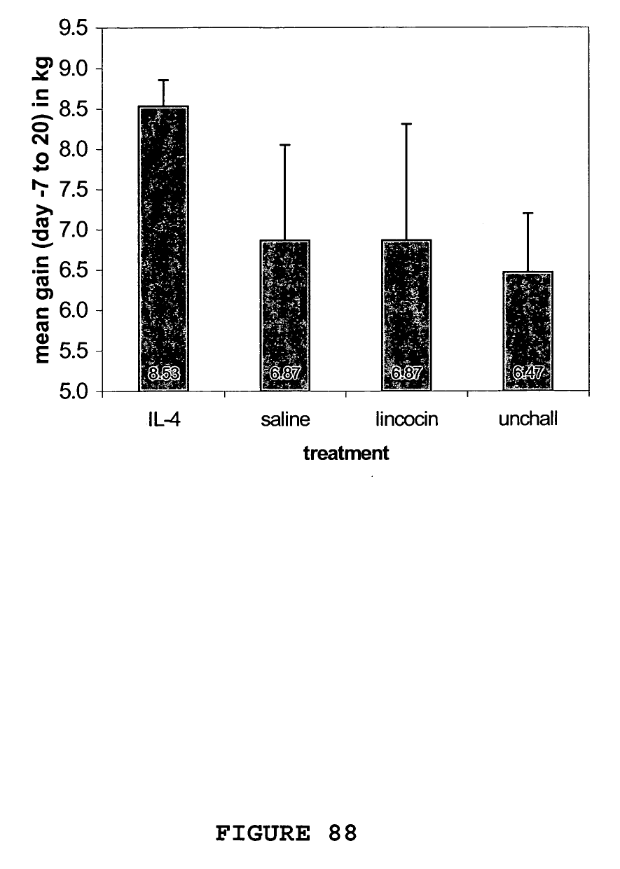

Further to reducing the severity of swine dysentery infection in pigs, treatment with IL-4 was able to improve the growth rate of pigs during the challenge phase (FIG. 86) final slaughter weight (FIG. 87) and weight gained (FIG. 88). Prior to challenge or treatment, groups show the same mean weight of 6.5 kg (FIG. 86, day −7). By the end of the trial on days 19 and 20, pigs treated with IL-4 weighed 15.1 kg compared with 13.6 kg for saline treated pigs (FIG. 87), an improvement of 11% in end weight. Comparably, at the end of the trial, the weight of pigs treated with Lincocin was 12.2 kg, and the weight of unchallenged pigs was 12.1 kg.

Total weight gained over the challenge period for pigs treated with IL-4 was 8.5 kg compared with 6.9 kg for both saline and Lincomycin treatments and 6.5 kg for unchallenged pigs (FIG. 88). The improvement in gain over the trial period produced by IL-4 treatment compared to saline or antibiotic treatment was 24%, from day 7 prior to challenge to slaughter at days 19 and 20 (FIG. 88). Unexpectedly, unchallenged pigs showed the poorest growth performance of all groups, which may be the result of their being housed in a separate room to prevent cross-contamination, and thus, room effects cannot be eliminated. Although there were no significant differences between treatment groups for weight, treatment with IL-4 did reduce variability by increasing the weight gained by smaller pigs. In previous experience with challenge models, and indeed in field situations, the pigs most susceptible to infection tend to be of lower weight. The ability of IL-4 to lift the weight of smaller pigs under challenge conditions may be able to reduce the susceptibility of these smaller animals to infections seen in commercial conditions.

### Conclusions

Treatment of pigs with IL-4 significantly reduced the number of spirochaetes present in the hindgut and faeces at postmortem compared with saline treatment. IL-4 reduced the clinical manifestation of swine dysentery infection as detected by faecal condition, compared with saline controls.

Pigs treated with IL-4 or Lincocin showed reduced signs of gross pathology normally associated with swine dysentery compared with saline treated pigs.

The improvement in health, as determined by reduced clinical signs, reduced pathology and reduced presence of spirochaetes in the gut and faeces, with IL-4 treatment, was comparable to the results obtained using the antibiotic Lincocin, the current therapeutic for swine dysentery infection in pig herds.

Treatment of pigs with IL-4 resulted in improved growth compared to all other treatment groups. At the end of the trial, pigs treated with IL-4 were 12% heavier than their saline treated counterparts, and 15% heavier than pigs treated with Lincocin.

Weight gained over the experimental period was 24% higher in the IL-4 treated group compared to saline or Lincocin treatments; the increase in weight was most notable in pigs with a smaller starting weight.

IL-4 as prophylactics to improve the growth and health of pigs exposed to infection.

IL-4 has been shown to improve the health of pigs in two infection models: Actinobacillus pleuropneumoniae (App) and Brachyspira (Serpulina) hyodysenteriae (swine dysentery). Improvements in health in both models were described by reduced clinical symptoms during infection and a reduction in infection associated pathology at postmortem. In the swine dysentery model, reduced spirochaete shedding was also noted. The ability of prophylactic treatment with IL-4 to improve the health of pigs was comparable to the performance of the current industry standards of antibiotic treatment. Thus, IL-4 has potential as an alternative, or supplement with, treatment to antibiotics, or preventative, for App and swine dysentery in pigs. The potential of IL-4 as a health promoter may be further enhanced by concurrent application with antibiotic therapeutics.

Furthermore, IL-4 was shown to improve the growth performance of pigs under both disease challenge models. This effect was not seen in the groups administered with the current therapeutic antibiotics used to treat these infections. Thus, IL-4 displays not only health promoting properties, but also growth promoting potential.

The claims defining the invention are as follows

1. A method for improving the growth performance of an animal comprising the step of administering to an animal in need thereof a growth promoting amount of one or more cytokines or biologically-active fragments thereof.

2. A method according to claim 1, wherein the cytokine or fragment thereof is administered optionally in combination with a pharmaceutical carrier, adjuvant or vehicle.

3. A method for improving the growth performance of an animal comprising the step of administering to an animal in need thereof a composition comprising a cytokine or biologically-active fragment thereof in conjunction with an antibiotic, optionally in combination with a pharmaceutical carrier, adjuvant or vehicle, wherein said composition achieves a synergistic growth promoting effect.

4. A method for improving the growth performance of an animal comprising the step of administering to an animal in need thereof a compound or composition which increases or supplements endogenous cytokine levels such that a growth promoting amount of one or more cytokines is produced, wherein growth performance is enhanced relative to the growth performance of an animal which has not been administered said compound or composition.

5. A method according to claim 4, wherein the compound or composition is administered prior to, together with, or subsequent to the administration of a growth promoting amount of one or more cytokines.

6. A method according to any one of claims 1 to 3 or 5, wherein the cytokine is selected from the group consisting of interleukin 1 (IL-1), interleukin 2 (IL-2), interleukin 3 (IL-3), interleukin 4 (IL-4), interleukin 5 (IL-5), interleukin 6 (IL-6), interleukin 7 (IL-7), interleukin 10 (IL-10), interleukin 11 (IL-11), interleukin 12 (IL-12), interleukin 13 (IL-13), granulocyte macrophage-colony stimulating factor (GM-CSF), granulocyte colony stimulating factor (G-CSF), macrophage-colony stimulating factor (M-CSF), erythropoietin (Epo), stem cell factor (SCF), leucocyte inhibitor factor (LIF), tumour growth factor beta (TGFβ) and tumour necrosis factor alpha (TNFα).

7. A method according to claim 6, wherein the cytokine is selected from the group consisting of interleukin 3 (IL-3), interleukin 4 (IL-4), interleukin 5 (IL-5) and granulocyte macrophage-colony stimulating factor (GM-CSF).

8. A method according to claim 6, wherein the cytokine is either interleukin 3 (IL-3), interleukin 4 (IL-4) or interleukin 5 (IL-5).

9. A method according to any one of claims 3, 6 to 8, wherein the administration of the antibiotic is prior to or subsequent to the administration of the cytokine.

10. A method according to any one of claims 1, 2, 4 or 5, further comprising the step of administering an antibiotic.

11. A method according to any one of claims 6 to 10, wherein the antibiotic is selected from the group consisting of amoxycylin, penicillin, procaine, ampicillin, cloxacillin, penicillin G, benzathine, benethamine, ceftiofur, cephalonium, cefuroxime, erythromycin, tylosin, tilmicosin, oleandomycin, kitasamycin, lincomycin, spectinomycin, tetracycline, oxytetracycline, chlortetracycline, neomycin, apramycin, streptomycin, avoparcin, dimetridazole, sulfonamides (including trimethoprim and diaveridine), bacitracin, virginiamycin, monensin, salinomycin, lasalocid, narasin and olaquindox or combinations thereof.

12. A method according to claim 11, wherein the antibiotic is either lincomycin, spectinomycin or amoxicillin or combinations thereof.

13. A method according to any one of claims 1 to 12, wherein the administration is orally, topically, or parenterally.

14. A method according to claim 13, wherein parenteral administration is either by subcutaneous injection, aerosol, intravenous, intramuscular, intrathecal, intrasternal injection, infusion techniques or encapsulated cells.

15. A method according to any one of claims 1 to 14, wherein the administration is either a single dose unit or a multiple dose unit.

16. A method according to any one of claims 1 to 13, wherein the administration is orally as an additive in water and/or feed.

17. A method according to any one of claims 1 to 16, wherein the growth performance of an animal is selected from the group consisting of an increase in growth rate, an increase in efficiency of feed use, an increase in final weight, an increase in dressed weight and decrease in fat content.

18. A method according to any one of claims 1 to 17, wherein the improved growth performance of an animal results from immunoenhancement, anti-parasitic or anti-microbial effects, anti-inflammatory effects or stress reduction.

19. A method according to any one of claims 1 to 17, wherein the animal is either an Artiodactyl or avian.

20. A method according to claim 19, wherein the Artiodactyl is selected from the group consisting of cattle, pigs, sheep, camels, goats and horses.

21. A method according to claim 19, wherein the avian is selected from the group consisting of chickens, turkeys, geese and ducks

22. A method according to claim 18, wherein the animal is either cattle, pigs, or sheep.

23. A growth promoting composition comprising one or more cytokines or biologically-active fragments thereof and one or more antibiotics.

24. A growth promoting composition according to claim 23, further comprising a pharmaceutical carrier, adjuvant or vehicle.

25. A growth promoting composition according to claim 23 or claim 24, wherein the cytokine is selected from the group consisting of interleukin 1 (IL-1), interleukin 2 (IL-2), interleukin 3 (IL-3), interleukin 4 (IL-4), interleukin 5 (IL-5), interleukin 6 (IL-6), interleukin 7 (IL-7), interleukin 10 (IL-10), interleukin 11 (IL-11), interleukin 12 (IL-12), interleukin 13 (IL-13), granulocyte macrophage-colony stimulating factor (GM-CSF), granulocyte colony stimulating factor (G-CSF), macrophage-colony stimulating factor (M-CSF), erythropoietin (Epo), stem cell factor (SCF), leucocyte inhibitor factor (LIF), tumour growth factor beta (TGFβ) and tumour necrosis factor alpha (TNFα).

26 A growth promoting composition according to claim 25, wherein the cytokine is selected from the group consisting of interleukin 3 (IL-3), interleukin 4 (IL-4), interleukin 5 (IL-5) and granulocyte macrophage-colony stimulating factor (GM-CSF).

27. A growth promoting composition according to claim 25, wherein the cytokine is either interleukin 3 (IL-3), interleukin 4 (IL-4) or interleukin 5 (IL-5).

28. A growth promoting composition according to any one of claims 23 to 27, wherein the antibiotic is selected from the group consisting of amoxycylin, penicillin, procaine, ampicillin, cloxacillin, penicillin G, benzathine, benethamine, ceftiofur, cephalonium, cefuroxime, erythromycin, tylosin, tilmicosin, oleandomycin, kitasamycin, lincomycin, spectinomycin, tetracycline, oxytetracycline, chlortetracycline, neomycin, apramycin, streptomycin, avoparcin, dimetridazole, sulfonamides (including trimethoprim and diaveridine), bacitracin, virginiamycin, monensin, salinomycin, lasalocid, narasin and olaquindox or combinations thereof.

29. A growth promoting composition according to claim 28, wherein the antibiotic is lincomycin, spectinomycin or amoxicillin or combinations thereof.

30. A method for improving the growth performance of an animal comprising the step of administering to an animal in need thereof a nucleic acid molecule encoding one or more cytokines or biologically-active fragments thereof, wherein the expression of said nucleic acid molecule produces a growth promoting amount of said cytokines or fragments thereof.

31. A method according to claim 30, wherein the nucleic acid molecule is administered by injection subcutaneously, intravenously, or intramuscularly or administered as an aerosol.

32. A method according to claim 30, wherein the nucleic acid molecule is administered in an amount of about 1 μg to 2000 μg per dose

33. A method according to claim 30, wherein the nucleic acid molecule is administered in an amount of about 5 μg to 1000 μg per dose.

34. A method according to claim 30, wherein the nucleic acid molecule is administered in an amount of about 6 μg to 200 μg per dose.

35. A method according to claim 27, wherein the nucleic acid molecule is administered in a vector or as naked DNA.

36. A method according to claim 35, wherein the vector is a porcine adenovirus vector.

37. A construct for delivering in vivo an effective amount of cytokine, comprising:
a) a nucleotide sequence encoding a cytokine or a biologically active fragment thereof;
b) a vector comprising a control sequence wherein the control sequence is-capable of the controlling the expression of the nucleotide sequence of a) such that a cytokine or biologically active fragment thereof is produced which in turns improves growth performance in an animal.
